(19) United States
(12) Patent Application Publication
(10) Pub. No.: US 2023/0314445 A1
(43) Pub. Date: Oct. 5, 2023

(54) METHODS FOR IDENTIFICATION OF ANTIGEN-BINDING MOLECULES

(71) Applicant: 10x Genomics, Inc., Pleasanton, CA (US)

(72) Inventors: Wyatt James MCDONNELL, Concord, CA (US); Michael John Terry STUBBINGTON, Cambridge (GB); Geoffrey P. MCDERMOTT, Alfredton (AU); David Michael PATTERSON, Pleasanton, CA (US)

(21) Appl. No.: 18/052,537

(22) Filed: Nov. 3, 2022

Related U.S. Application Data
(63) Continuation of application No. PCT/US2021/030976, filed on May 5, 2021.
(60) Provisional application No. 63/020,490, filed on May 5, 2020.
(60) Provisional application No. 63/152,763, filed on Feb. 23, 2021.
(60) Provisional application No. 63/152,769, filed on Feb. 23, 2021.

(51) Int. Cl. G01N 33/68; G01N 33/58

(52) U.S. Cl. CPC G01N 33/6854; G01N 33/58; G01N 2800/52

(57) ABSTRACT

Provided herein are methods for identification of antigen binding molecules such as antibodies from a sample by exposing the antigen binding molecules to an antigen conjugated to an oligonucleotide.

### CROSS-REFERENCE TO RELATED APPLICATIONS

This application claims priority to U.S. Provisional Patent Application Nos. 63/020,490, filed May 5, 2020, 63/152,769, filed Feb. 23, 2021, and 63/152,763, filed Feb. 23, 2021, the disclosures of which are incorporated by reference herein in their entireties, including any drawings.

### BACKGROUND

A sample can be processed for various purposes, such as identification of a type of moiety within the sample. The sample can be a biological sample. Biological samples can be processed, such as for detection of a disease (e.g., cancer) or identification of a particular species. There are various approaches for processing samples, such as polymerase chain reaction (PCR) and sequencing.

Biological samples can be processed within various reaction environments, such as partitions. Partitions can be wells or droplets. Droplets or wells can be employed to process biological samples in a manner that enables the biological samples to be partitioned and processed separately. For example, such droplets can be fluidically isolated from other droplets, enabling accurate control of respective environments in the droplets.

Biological samples in partitions can be subjected to various processes, such as chemical processes or physical processes. Samples in partitions can be subjected to heating or cooling, or chemical reactions, such as to yield species that can be qualitatively or quantitatively processed.

Drug-reactive antibodies (DRAs), also referred to interchangeably herein as anti-drug antibodies (ADAs), can be antibodies that develop from adaptive immune recognition of antibody-based drugs or therapies. The development of drug-reactive antibodies (DRAs) or anti-drug antibodies (ADAs) can be hazardous for patients on antibody-based therapies and severely inhibit or prevent drug efficacy. Anti-drug antibodies can be found in individuals who have never been exposed to a drug. Anti-drug antibodies can be characterized into three classes—(1) a paratope-specific, inhibitory, neutralizing, anti-idiotypic antibody; (2) an anti-idiotypic, non-paratope-specific, non-inhibitory; and (3) a drug-target complex specific, non-inhibitory antibody. Presence of any of these anti-drug antibody can contribute to poor outcomes. Thus, identifying these drug-reactive antibodies (DRAs) or anti-drug antibodies (ADAs) is an essential part of all clinical trials of antibodies and antibody-based therapies. Furthermore, a better understanding the drug-reactive antibody repertoire can guide drug development and even drug discovery. There is a clear need for efficiently identifying drug-reactive antibodies (DRAs) or anti-drug antibodies (ADAs).

### SUMMARY

Provided herein are methods for identifying an antigen binding molecule, said methods including contacting a composition including at least one antigen binding molecule with an antigen conjugated to a reporter oligonucleotide; isolating said antigen binding molecule bound to said antigen at least by using said oligonucleotide at least by using the reporter oligonucleotide; and identifying said antigen binding molecule.

In some embodiments, said composition includes at least one T cell. In some embodiments, said composition includes at least one B cell.

In some embodiments, said antigen binding molecule includes an antibody or antigen binding fragment thereof. In some embodiments, said at least one antigen binding molecule includes a monoclonal antibody. In some embodiments, said at least one antigen binding molecule includes a polyclonal antibody. In some embodiments, said at least one antigen binding molecule includes an antibody fragment. In some embodiments, said antibody fragment is selected from the group consisting of a Fab, an Fab′, an F(ab′)2, an Fv (e.g., a scFv), and an Fc. In some embodiments, said at least one antigen binding molecule includes an immunoglobulin. In some embodiments, said immunoglobulin is selected from the group consisting of IgA, IgD, IgE, IgG, and IgM.

In some embodiments, said antigen is a second antibody or antigen binding fragment. In some embodiments, said second antibody or antigen binding fragment includes a therapeutic antibody or antigen binding fragment thereof. In some embodiments, said therapeutic antibody or antigen binding fragment thereof is selected from the group consisting of abciximab, adalimumab, adalimumab-atto, ado-trastuzumab emtansine, alemtuzumab, alirocumab, atezolizumab, avelumab, basiliximab, belimumab, bevacizumab, bezlotoxumab, blinatumomab, brentuximab vedotin, brodalumab, canakinumab, capromab pendetide, certolizumab pegol, cetuximab, daclizumab, daratumumab, denosumab, dinutuximab, dupilumab, durvalumab, eculizumab, elotuzumab, evolocumab, golimumab, ibritumomab tiuxetan, idarucizumab, infliximab, infliximab-abda, infliximab dyyb, ipilimumab, ixekizumab, mepolizumab, natalizumab, necitumumab, nivolumab, oblitoxaximab, obinutuzumab, ocrelizumab, ofatumumab, olaratumab, omalizumab, palivizumab, panitumumab, pembrolizumab, pertuzumab, ramucirumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, adalimumab-adbm, gemtuzumab ozogamicin, bevacizumab-awwb, benralizumab, emicizumab-kxwh, trastuzumab-dkst, infliximab-qbtx, ibalizumab-uiyk, tildrakizumab-asmn, burosumab-twsa, erenumab-aooe, tositumomab, mogamulizumab, moxetumomab pasudotox, cempilimab, polatuzumab vedotin, idecabtagene vicleucel, lisocabtagene maraleucel, brexucabtagene autoleucel, tisagenlecleucel, and axicabtagene ciloleucel. In some embodiments, said therapeutic antibody or antigen binding fragment thereof is an anti-SARS-CoV-2 antibody or antigen binding fragment thereof. In some embodiments, said second antibody or antigen binding fragment includes a monoclonal antibody. In some embodiments, said second antibody or antigen binding fragment includes a polyclonal antibody. In some embodiments, said second antibody or antigen binding fragment includes an antibody fragment. In some embodiments, said antibody fragment is selected from the group consisting of a Fab, an Fab′, an F(ab′)2, an Fv (e.g., a scFv), and an Fc. In some embodiments, said second antibody or antigen binding fragment includes an immunoglobulin. In some embodiments, said immunoglobulin is selected from the group consisting of IgA, IgD, IgE, IgG, and IgM. In some embodiments, said second antibody or antigen binding fragment includes a bispecific antibody. In some embodiments, said antigen is a component of a vaccine. In some embodiments, said antigen is a small molecule.

In some embodiments, said reporter oligonucleotide comprises a reporter barcode sequence. In some embodiments, said reporter oligonucleotide is conjugated to a constant region of said antigen. In some embodiments, said reporter oligonucleotide is conjugated to a variable region of said antigen.

In some embodiments, said isolating comprises capturing said reporter oligonucleotide. In some embodiments, said capturing includes annealing said reporter oligonucleotide to a second oligonucleotide (e.g., a partition-specific barcode molecule).

In some embodiments, the method further includes pulling down said at least one antigen binding molecule using said second oligonucleotide (e.g., a partition-specific barcode molecule). In some embodiments, said second oligonucleotide (e.g., a partition-specific barcode molecule) is releasably attached to a gel bead. In some embodiments, second oligonucleotide (e.g., a partition-specific barcode molecule) is affixed to a slide. In some embodiments, said second oligonucleotide (e.g., a partition-specific barcode molecule) is affixed to a well. In some embodiments, said second oligonucleotide (e.g., a partition-specific barcode molecule) is affixed to a tube. In some embodiments, said second oligonucleotide (e.g., a partition-specific barcode molecule) is conjugated to a magnetic molecule. In some embodiments, said second oligonucleotide (e.g., a partition-specific barcode molecule) includes a unique molecular identifier. In some embodiments, said second oligonucleotide (e.g., a partition-specific barcode molecule) includes a template switching oligonucleotide.

In some embodiments, the method further includes identifying a site on said antigen binding to said antigen binding molecule.

In some embodiments, the method further includes modifying said antigen to reduce affinity of said antigen binding molecule for said antigen.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Provided herein is a method of identifying an antigen binding molecule, the method including: contacting a composition including at least one antigen binding molecule with an antigen conjugated to a reporter oligonucleotide; isolating the antigen binding molecule bound to the antigen at least by using the reporter oligonucleotide; and identifying the antigen binding molecule.

Provided herein is a method of identifying an antigen binding molecule that binds an antigen, the method including: contacting a reporter oligonucleotide conjugated antigen to one or more cells expressing the antigen binding molecule, wherein the reporter oligonucleotide includes a reporter barcode and the antigen is an antibody therapeutic; sequencing the antigen binding molecule; and identifying the antigen binding molecule. In some embodiments, the method further includes isolating the antigen binding molecule.

Provided herein is a method of identifying an antigen binding molecule that binds an antigen, the method including: contacting a reporter oligonucleotide conjugated antigen to one or more cells expressing the antigen binding molecule, wherein the reporter oligonucleotide includes a reporter barcode and the antigen is an antibody drug conjugate (ADC); sequencing the antigen binding molecule; and identifying the antigen binding molecule. In some embodiments, the method further includes isolating the antigen binding molecule.

Provided herein is a method of quantifying a subject's response to an antigen, the method including: contacting (i) a composition including one or more cells from the subject wherein the one or more cells express at least one antigen binding molecule with (ii) a reporter oligonucleotide conjugated antigen, wherein the reporter oligonucleotide includes a reporter barcode and wherein the antigen is an antibody therapeutic; measuring the number of cells that express the at least one antigen binding molecule that bind to the antibody therapeutic. In an embodiment, the method is not used for a diagnostic purpose.

Provided herein is a method of quantifying a subject's response to an antigen, the method including: contacting (i) a composition including one or more cells from the subject wherein the one or more cells express at least one antigen binding molecule with (ii) a reporter oligonucleotide conjugated antigen, wherein the reporter oligonucleotide includes a reporter barcode and wherein the antigen is an antibody drug conjugate (ADC); measuring the number of cells that express the at least one antigen binding molecule that bind to the antibody drug conjugate (ADC). In an embodiment, the method is not used for a diagnostic purpose.

Provided herein is a method of determining diversity of a subject's immune response to an antigen, the method including: contacting (i) a composition including one or more cells from the subject wherein the one or more cells express one or more antigen binding molecules with (ii) a reporter oligonucleotide conjugated antigen, wherein the reporter oligonucleotide includes a reporter barcode and wherein the antigen is an antibody therapeutic; sequencing the one or more antigen binding molecules; and identifying the one or more antigen binding molecules to determine the diversity of a subject's immune response to the antibody therapeutic. In some embodiments, the method further includes isolating the one or more antigen binding molecules. In some embodiments, the diversity of a subject's immune response is production of different antibodies and/or antibody lineages. In an embodiment, the method is not used for a diagnostic purpose.

Provided herein is a method of determining diversity of a subject's immune response to an antigen, the method including: contacting (i) a composition including one or more cells from the subject wherein the one or more cells express one or more antigen binding molecules with (ii) a reporter oligonucleotide conjugated antigen, wherein the reporter oligonucleotide includes a reporter barcode and wherein the antigen is an antibody drug conjugate (ADC); sequencing the one or more antigen binding molecules; and identifying the one or more antigen binding molecules to determine the diversity of a subject's immune response to the antibody drug conjugate (ADC). In some embodiments, the method further includes isolating the one or more antigen binding molecules. In some embodiments, the diversity of a subject's immune response is production of different antibodies and/or antibody lineages. In an embodiment, the method is not used for a diagnostic purpose.

Provided herein is a method of monitoring a subject's response to an antibody therapeutic or antibody drug conjugate (ADC), the method including: contacting (i) a composition comprising one or more cells from the subject wherein the one or more cells express one or more antigen binding molecules with (ii) a reporter oligonucleotide conjugated antigen, wherein the reporter oligonucleotide comprises a reporter barcode and wherein the antigen is an antibody therapeutic or ADC; identifying the one or more antigen binding molecules; and measuring the number of cells that express at least one antigen binding molecule that bind to the antibody therapeutic or ADC over a course of time. In an embodiment, the method is not used for a diagnostic purpose. In some embodiments, the method further includes isolating the one or more antigen binding molecule. In some embodiments, the course of time is about bi-weekly, about monthly, or about yearly. In some embodiments, the one or more cells comprises at least one NK cell. In some embodiments, the one or more cells includes at least one B cell. In some embodiments, the one or more cells are obtained from a subject previously treated with the antibody therapeutic. In some embodiments, the antigen binding molecule includes an antibody or antigen binding fragment thereof. In some embodiments, the sequencing step includes determining all or a part of the sequence of the antibody or antigen binding fragment thereof. In some embodiments, the antibody or antigen binding fragment thereof is an anti-SARS-CoV-2 antibody or antigen binding fragment thereof.

In some embodiments, the antibody is a monoclonal antibody. In some embodiments, the antibody is a polyclonal antibody. In some embodiments, the antibody is a bispecific antibody. In some embodiments, the antibody is a secreted antibody. In some embodiments, the antibody is a surface-bound antibody. In some embodiments, the antigen binding fragment is selected from the group consisting of a Fab, an Fab′, an F(ab′)2, an Fv, and an Fc. In some embodiments, the antigen is a component of a vaccine. In some embodiments, the antigen is a chimeric antigen receptor. In some embodiments, the chimeric antigen receptor is selected from the group consisting of axicabtagene ciloleucel (YESCARTA®), tisagenlecleucel (KYMRIAH®), brexucabtagene autoleucel (TECARTUS), idecabtagene vicleucel (ABECMA®), and lisocabtagene maraleucel. (BREYANZI®).

In some embodiments, the reporter oligonucleotide conjugated antigen further includes an enzyme tag, a fluorophore tag, a quantum dot, a covalently or non-covalently attached protein tag, a covalently or non-covalently attached peptide tag, a fused tag, a carbohydrate tag, or a small molecule tag. In some embodiments, the covalently or non-covalently attached protein tag is selected from BCCP (biotin carboxyl carrier protein) tag, glutathione-S-transferase tag, green fluorescent protein tag, halo-tag, SNAP tag, CLIP tag, HUH tag, maltose binding protein tag, Nus tag, thioredoxin tag, Fc tag, and CRDSAT tag. In some embodiments, the covalently or non-covalently attached peptide tag is selected from ALFA tag, AviTag, C-tag, calmodulin tag, polyglutamate tag, polyarginine tag, E tag, FLAG tag, HA tag, His tag, Myc tag, NE tag, Rho1D4 tag, S tag, SBP tag, Softag 1, Softag 3, Spot tag, Strep tag, T7 tag, TC tag, Ty tag, V5 tag, VSV tag, Xpress tag, Isopeptag, Spy tag, Snoop tag, DogTag, and SdyTag.

In some embodiments, the conjugating the antibody therapeutic to the oligonucleotide includes one or more of the following: ReACT chemistry, direct/non-specific (lysine) click chemistry, site-specific sortase motif-dependent conjugation, site-specific photo-crosslinking-dependent conjugation, site-specific conformation-dependent conjugation, and nitrilotriacetate conjugation.

In some embodiments, the reporter oligonucleotide is coupled to a constant region of the antibody. In some embodiments, the isolating step of the methods provided herein includes capturing the reporter oligonucleotide. In some embodiments, the isolating step further comprises partitioning. In some embodiments, the partitioning generates a plurality of partitions. In some embodiments, a partition of the plurality of partitions includes a single cell. In some embodiments, the partition of the plurality of partitions includes a plurality of partition-specific barcode molecules including a partition-specific barcode sequence. In some embodiments, the methods provided herein further includes using a partition-specific barcode molecule of the plurality of partition-specific barcode molecules and an analyte of the single cell to generate a barcoded analyte of the single cell. In some embodiments, the barcoded analyte is a RNA, DNA, polypeptide, protein, or a combination thereof. In some embodiments, the capturing includes contacting the reporter oligonucleotide including the reporter barcode to a partition-specific barcode molecule. In some embodiments, the contacting comprises hybridizing the reporter oligonucleotide to the partition-specific barcode molecule.

In some embodiments, the partition-specific barcode molecule hybridized to the reporter oligonucleotide is releasably attached or coupled to a solid support. In some embodiments, the solid support is selected from the group consisting of a bead, a microwell, a glass slide, a tube, a microfluidic chip, or a flow cell. In some embodiments, the solid support is a gel bead. In some embodiments, the partition-specific barcode molecule is released from the gel bead prior to generating a nucleic acid barcode molecule. In some embodiments, the partition-specific barcode molecule is coupled to a magnetic-sensitive matrix.

In some embodiments, the partition-specific barcode molecule includes a partition-specific barcode, a unique molecular identifier (UMI), and/or a template switching oligonucleotide (TSO) site. In some embodiments, the UMI of the partition-specific barcode molecule differs from the UMI of another partition-specific barcode molecule of the plurality of partition-specific barcode molecules in the partition. In some embodiments, the partition-specific barcode molecule further includes a functional sequence. In some embodiments, the partition-specific barcode molecule further includes a capture sequence hybridizing to the reporter oligonucleotide. In some embodiments, the capture sequence includes a polyT sequence. In some embodiments, the capture sequence is complementary to a capture handle sequence of the reporter oligonucleotide.

In some embodiments, the antibody therapeutic is selected from the group consisting of abciximab, adalimumab, adalimumab-atto, ado-trastuzumab emtansine, alemtuzumab, alirocumab, atezolizumab, avelumab, basiliximab, belimumab, bevacizumab, bezlotoxumab, blinatumomab, brentuximab vedotin, brodalumab, canakinumab, capromab pendetide, certolizumab pegol, cetuximab, daclizumab, daratumumab, denosumab, dinutuximab, dupilumab, durvalumab, eculizumab, elotuzumab, evolocumab, golimumab, ibritumomab tiuxetan, idarucizumab, infliximab, infliximab-abda, infliximab dyyb, ipilimumab, ixekizumab, mepolizumab, natalizumab, necitumumab, nivolumab, oblitoxaximab, obinutuzumab, ocrelizumab, ofatumumab, olaratumab, omalizumab, palivizumab, panitumumab, pembrolizumab, pertuzumab, ramucirumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, adalimumab-adbm, gemtuzumab ozogamicin, bevacizumab-awwb, benralizumab, emicizumab-kxwh, trastuzumab-dkst, infliximab-qbtx, ibalizumab-uiyk, tildrakizumab-asmn, burosumab-twsa, erenumab-aooe, tositumomab, mogamulizumab, moxetumomab pasudotox, cempilimab, polatuzumab vedotin, idecabtagene vicleucel, lisocabtagene maraleucel, brexucabtagene autoleucel, tisagenlecleucel, and axicabtagene ciloleucel.

Provided herein is a method of characterizing one or more cells expressing one or more antigen binding molecules that bind to an antigen, the method including: contacting the one or more cells expressing the one or more antigen binding molecules to at least one reporter oligonucleotide conjugated antigen, wherein an reporter oligonucleotide includes a reporter barcode and the antigen is an antibody therapeutic; and using the reporter barcode to identify the one or more cells binding to the at least one reporter oligonucleotide conjugated antigens, and optionally isolate the one or more cells binding to the at least one reporter oligonucleotide conjugated antigens; and using the binding to generate a count matrix comprising information for (i) the binding cell counts and/or (ii) unique molecular identifier (UMI) counts. In some embodiments, the method further includes embedding, in a lower dimensional space, the count matrix, the embedding includes transforming the count matrix by applying one or more of a log-transformation, a variance-stabilizing transformation, a square root transformation, and a cubic root transformation; and generating, based at least on the embedded count matrix, to identify one or more distinct populations, wherein each population of the one or more distinct populations represents a similar binding profile.

Provided herein is a method of isolating an anti-drug antibody, the method comprising: (a) contacting a composition comprising one or more cells from a subject treated with an antibody therapeutic to a reporter oligonucleotide conjugated antigen, wherein the reporter oligonucleotide comprises a reporter barcode and the antigen is the antibody therapeutic; and (b) using the reporter barcode to identify the one or more cells binding to the reporter oligonucleotide conjugated antigen, and optionally isolating the one or more cells binding to the reporter oligonucleotide conjugated antigen. In some embodiments, the method further comprises identifying an antigen binding site of the antibody therapeutic to which the anti-drug antibody binds. In some embodiments, the method optionally comprises modifying the antigen binding site of the antibody therapeutic to modify the binding of the antibody therapeutic to the anti-drug antibody.

Provided herein is a method of identifying an antibody therapeutic that does not elicit an immune response, the method including: contacting a composition comprising one or more cells from a subject treated with the antibody therapeutic to (i) a control reporter oligonucleotide conjugated antigen, wherein the control reporter oligonucleotide comprises a first reporter barcode and the antigen is the antibody therapeutic, and (ii) at least one test reporter oligonucleotide conjugated antigen, wherein the test reporter oligonucleotide comprises a second reporter barcode and the antigen is a modified version of the antibody therapeutic; and using the first and second reporter barcodes to identify the one or more cells binding to (i) and/or (ii); and identifying a test reporter oligonucleotide conjugated antigen of the at least one test reporter oligonucleotide conjugated antigen, wherein the test reporter oligonucleotide conjugated antigen has less binding as compared to the control reporter oligonucleotide conjugated antigen. In some embodiments, the method optionally includes isolating the one or more cells binding to (i) and/or (ii). In some embodiments, the method further includes using the binding to generate a count matrix comprising information for the binding cell counts and/or unique molecular identifier (UMI) counts. In some embodiments, the method further includes embedding, in a lower dimensional space, the count matrix, the embedding includes transforming the count matrix by applying one or more of a log-transformation, a variance-stabilizing transformation, a square root transformation, and a cubic root transformation; and generating, based at least on the embedded count matrix, to identify one or more distinct populations, wherein each population of the one or more distinct populations represents a similar binding profile. In some embodiments, the modified version of the antibody therapeutic includes at least one point mutation. In some embodiments, the method further includes identifying an antigen binding site in which the modified version of the antibody therapeutic binds. In some embodiments, the method further includes identifying an epitope on an antibody therapeutic recognized by an anti-drug antibody conditional on binding of the antibody therapeutic's antigenic target (AgTx). In some embodiments, the method further includes identifying a paratope on an antibody therapeutic or modified (mutated) antibody therapeutic, which binds to the AgTx. In some embodiments, the method further includes identifying an epitope on an antibody therapeutic recognized by an anti-drug antibody independent of binding of the antibody therapeutic's antigenic target (AgTx). In some workflows, simultaneous binding or lack thereof of the antibody therapeutic to its antigenic target (AgTx) and one or more anti-drug antibodies can be used to assess the successful deimmunizing of the antibody therapeutic. In some embodiments, the modified version of the antibody therapeutic is used or is capable of being used as a subject-specific antibody therapeutic. In some embodiments, the antibody therapeutic is bound or unbound to its target antigen.

Provided herein is a system, including: at least one data processor; and at least one memory storing instructions, which when executed by the at least one data processor, result in operations including: generating, based at least on a reporter oligonucleotide conjugated to each of a plurality of antigens, a count matrix indicating a count of a quantity of times each of the plurality of antigens bound to each of a plurality of cells expressing one or more antigen binding molecules; embedding, in a lower dimensional space, the count matrix; and identifying, based at least on the embedded count matrix, one or more distinct populations of cells expressing one or more antigen binding molecules, each of the one or more distinct populations of cells expressing one or more antigen binding molecules capable of binding to one or more of a same antigen. In some embodiments, the reporter oligonucleotide includes a reporter barcode. In some embodiments, the reporter oligonucleotide is coupled with a partition-specific barcode molecule including one or more of a partition-specific barcode, a unique molecular identifier (UMI), and a template switching oligonucleotide (TSO) site. In some embodiments, the count matrix is embedded in the lower dimensional space by at least applying, to the count matrix, a transformation. In some embodiments, the transformation comprises one or more of a log-transformation, a variance-stabilizing transformation, a square root transformation, and a cubic root transformation. In some embodiments, a pseudocount is added to the count matrix prior to applying the transformation. In some embodiments, the pseudocount ranges from 1 to 30 or from 1 to 50. In some embodiments, the count matrix is scaled prior to applying the transformation. In some embodiments, the count matrix is scaled by a deviation or a covariance of one or more of the plurality of antigens. In some embodiments, the count matrix is denoised prior to applying the transformation. In some embodiments, the count matrix is denoised by at least fitting the count matrix to a mixture model.

In some embodiments, the mixture model includes one or more covariates including one or more of a quantity of detected reads, a quantity of detected genes, a quantity of detected gene unique molecular identifiers (UMIs), a quantity of detected antigens, a quantity of detected antigen unique molecular identifiers (UMIs), a quantity of detected surface or intracellular protein unique molecular identifiers, a quantity of detected ATAC peaks, a quantity of detected surface proteins, a quantity of detected intracellular proteins, B-cell phenotypes, an IgG constant region, a quantity of mutations in antibody sequence, sequencing depth, quantity of detected unique molecular identifiers (UMIs), and cell annotations.

In some embodiments, the operations further comprise: determining an ambient concentration of each of the plurality of antigens and/or the reporter oligonucleotide conjugated to each of the plurality of antigens; and subtracting, from the count matrix, the ambient concentration prior to embedding the count matrix. In some embodiments, the operations further include: filtering the count matrix to at least retain one or more cells expressing one or more antigen binding molecules having (i) a detected variability, diversity, and joining (VDJ) sequence and/or an antibody sequence, (ii) a non-zero binding count, and (iii) a sufficient sequencing depth. In some embodiments, the operations further include: generating a visualization of the one or more distinct populations of cells expressing one or more antigen binding molecules.

In some embodiments, the visualization is generated by at least generating a reduced dimension representation of the count matrix. In some embodiments, the reduced dimension representation of the count matrix is generated by applying one or more of a principal component analysis (PCA), uniform manifold approximation and projection (UMAP), T-distributed Stochastic Neighbor Embedding (t-SNE), and Poincare disks. In some embodiments, the visualization is further generated by applying, to the reduced dimension representation of the count matrix, one or more of a spectral clustering algorithm, a granularity-based community detection algorithm, and a resolution-based community detection algorithm.

Provided herein is a computer-implemented method, including: generating, based at least on a reporter oligonucleotide conjugated to each of a plurality of antigens, a count matrix indicating a count of a quantity of times each of the plurality of antigens bound to each of a plurality of cells expressing one or more antigen binding molecules; embedding, in a lower dimensional space, the count matrix; and identifying, based at least on the embedded count matrix, one or more distinct populations of cells expressing one or more antigen binding molecules, each of the one or more distinct populations of cells expressing one or more antigen binding molecules capable of binding to one or more of a same antigen. In some embodiments, the reporter oligonucleotide includes a reporter barcode. In some embodiments, the reporter oligonucleotide is coupled with a partition-specific barcode molecule comprising one or more of a partition-specific barcode, a unique molecular identifier (UMI), and a template switching oligonucleotide (TSO) site. In some embodiments, the count matrix is embedded in the lower dimensional space by at least applying, to the count matrix, a transformation. In some embodiments, the transformation includes one or more of a log-transformation, a variance-stabilizing transformation, a square root transformation, and a cubic root transformation. In some embodiments, a pseudocount is added to the count matrix prior to applying the transformation. In some embodiments, the pseudocount ranges from 1 to 30 or from 1 to 50. In some embodiments, the count matrix is scaled prior to applying the transformation. In some embodiments, the count matrix is scaled by a deviation or a covariance of one or more of the plurality of antigens. In some embodiments, the count matrix is denoised prior to applying the transformation. In some embodiments, the count matrix is denoised by at least fitting the count matrix to a mixture model. In some embodiments, the mixture model includes one or more covariates comprising one or more of a quantity of detected reads, a quantity of detected genes, a quantity of detected gene unique molecular identifiers (UMIs), a quantity of detected antigens, a quantity of detected antigen unique molecular identifiers (UMIs), a quantity of detected surface or intracellular protein unique molecular identifiers, a quantity of detected ATAC peaks, a quantity of detected surface proteins, a quantity of detected intracellular proteins, B-cell phenotypes, an IgG constant region, a quantity of mutations in antibody sequence, sequencing depth, quantity of detected unique molecular identifiers (UMIs), and cell annotations.

In some embodiments, the operations further comprise: determining an ambient concentration of each of the plurality of antigens and/or the reporter oligonucleotide conjugated to each of the plurality of antigens; and subtracting, from the count matrix, the ambient concentration prior to embedding the count matrix. In some embodiments, the operations further comprise: filtering the count matrix to at least retain one or more cells expressing one or more antigen binding molecules having (i) a detected variability, diversity, and joining (VDJ) sequence and/or an antibody sequence, (ii) a non-zero binding count, and (iii) a sufficient sequencing depth. In some embodiments, the operations further comprise: generating a visualization of the one or more distinct populations of cells expressing one or more antigen binding molecules. In some embodiments, the visualization is generated by at least generating a reduced dimension representation of the count matrix. In some embodiments, the reduced dimension representation of the count matrix is generated by applying one or more of a principal component analysis (PCA), uniform manifold approximation and projection (UMAP), T-distributed Stochastic Neighbor Embedding (t-SNE), and Poincare disks. In some embodiments, the visualization is further generated by applying, to the reduced dimension representation of the count matrix, one or more of a spectral clustering algorithm, a granularity-based community detection algorithm, and a resolution-based community detection algorithm.

Provided herein is a non-transitory computer readable medium storing instructions, which when executed by at least one data processor, result in operations comprising: generating, based at least on a reporter oligonucleotide conjugated to each of a plurality of antigens, a count matrix indicating a count of a quantity of times each of the plurality of antigens bound to each of a plurality of cells expressing one or more antigen binding molecules; embedding, in a lower dimensional space, the count matrix; and identifying, based at least on the embedded count matrix, one or more distinct populations of cells expressing one or more antigen binding molecules, each of the one or more distinct populations of cells expressing one or more antigen binding molecules capable of binding to one or more of a same antigen.

Provided herein is a method, including: contacting a plurality of antigens to a plurality of cells expressing one or more antigen binding molecules, wherein each of the plurality of antigens is conjugated to a reporter oligonucleotide comprising a reporter barcode, and wherein the antigen is an antibody therapeutic; identifying the plurality of cells expressing the one or more antigen binding molecules; generating, based at least on the reporter oligonucleotide conjugated to each of the plurality of antigens, a count matrix indicating a count of a quantity of times each of the plurality of antigens bound to each of the plurality of cells expressing one or more antigen binding molecules; embedding, in a lower dimensional space, the count matrix; and identifying, based at least on the embedded count matrix, one or more distinct populations of cells expressing one or more antigen binding molecules, each of the one or more distinct populations of cells expressing one or more antigen binding molecules capable of binding to one or more of a same antigen.

Provided herein is use of a reporter oligonucleotide conjugated antigen for quantifying a subject's response to the antigen, wherein the reporter oligonucleotide comprises a reporter barcode, wherein the antigen is an antibody therapeutic, and wherein the use comprises the steps of (a) contacting a composition comprising one or more cells from the subject wherein the one or more cells express at least one antigen binding molecule with the reporter oligonucleotide conjugated antigen; and (b) measuring the number of cells that express the at least one antigen binding molecule that bind to the antibody therapeutic.

Provided herein is use of a reporter oligonucleotide conjugated antigen for quantifying a subject's response to the antigen, wherein the reporter oligonucleotide comprises a reporter barcode, wherein the antigen is an antibody drug conjugate (ADC), and wherein the use comprises the steps of (a) contacting a composition comprising one or more cells from the subject wherein the one or more cells express at least one antigen binding molecule with the reporter oligonucleotide conjugated antigen; and (b) measuring the number of cells that express the at least one antigen binding molecule that bind to the antibody drug conjugate (ADC).

Provided herein is use of a reporter oligonucleotide conjugated antigen for determining diversity of a subject's immune response to the antigen, wherein the reporter oligonucleotide comprises a reporter barcode, wherein the antigen is an antibody therapeutic, and wherein the use comprises the steps of (a) contacting (i) a composition comprising one or more cells from the subject wherein the one or more cells express one or more antigen binding molecules with (ii) a reporter oligonucleotide conjugated antigen, wherein the reporter oligonucleotide comprises a reporter barcode and wherein the antigen is an antibody therapeutic; (b) sequencing the one or more antigen binding molecules; and (c) identifying the one or more antigen binding molecules that bind to the antibody therapeutic to determine the diversity of a subject's immune response to the antibody therapeutic. In some embodiments, the steps further comprise (d) isolating the one or more antigen binding molecules. In some embodiments, the diversity of the subject's immune response is production of different antibodies and/or antibody lineages.

Provided herein is use of a reporter oligonucleotide conjugated antigen for determining diversity of a subject's immune response to the antigen, wherein the reporter oligonucleotide comprises a reporter barcode, wherein the antigen is an antibody drug conjugate (ADC), and wherein the use comprises the steps of (a) contacting (i) a composition comprising one or more cells from the subject wherein the one or more cells express one or more antigen binding molecules with (ii) a reporter oligonucleotide conjugated antigen, wherein the reporter oligonucleotide comprises a reporter barcode and wherein the antigen is an antibody drug conjugate (ADC); (b) sequencing the one or more antigen binding molecules; and (c) identifying the one or more antigen binding molecules that bind to the antibody drug conjugate (ADC) to determine the diversity of a subject's immune response to the antibody drug conjugate (ADC). In some embodiments, the steps further comprise (d) isolating the one or more antigen binding molecules. In some embodiments, the diversity of the subject's immune response is production of different antibodies and/or antibody lineages.

Provided herein is use of a reporter oligonucleotide conjugated antigen for monitoring a subject's response to an antibody therapeutic or antibody drug conjugate, wherein the reporter oligonucleotide comprises a reporter barcode, wherein the antigen is an antibody therapeutic, and wherein the use comprises the steps of (a) contacting (i) a composition comprising one or more cells from the subject wherein the one or more cells express one or more antigen binding molecules with (ii) a reporter oligonucleotide conjugated antigen, wherein the reporter oligonucleotide comprises a reporter barcode and wherein the antigen is an antibody therapeutic or ADC; (b) identifying the one or more antigen binding molecules; and (c) measuring the number of cells that express at least one antigen binding molecule that bind to the antibody therapeutic or ADC over a course of time. In some embodiments, the steps further comprise (d) isolating the one or more antigen binding molecule. In some embodiments, the course of time is about bi-weekly, about monthly, or about yearly.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an exemplary microfluidic channel structure for partitioning individual biological particles or analyte careers.

FIG. 2 shows an exemplary microfluidic channel structure for delivering barcode carrying beads to droplets.

FIG. 3 shows an exemplary microfluidic channel structure for co-partitioning biological particles and reagents.

FIG. 4 shows an exemplary microfluidic channel structure for the controlled partitioning of beads into discrete droplets.

FIG. 5 shows an exemplary microfluidic channel structure for increased droplet generation throughput.

FIG. 6 shows an exemplary microfluidic channel structure for increased droplet generation throughput.

FIG. 7A shows a cross-section view of an exemplary microfluidic channel structure with a geometric feature for controlled partitioning. FIG. 7B shows a perspective view of the channel structure of FIG. 7A.

FIG. 8 illustrates an example of barcode (e.g., partition-specific barcode molecules) carrying bead.

FIG. 9 shows a computer system that is programmed or otherwise configured to implement methods provided herein.

FIG. 10 shows a labeling scheme for conjugating variations of an antigen with reporter oligonucleotides.

FIG. 11 shows a flow chart depicting a scheme for characterizing an immune response to a therapeutic antibody using methods provided herein.

FIG. 12A shows an example of an oligonucleotide (e.g., reporter oligonucleotide) conjugated to an antigen (or an antibody) hybridized to a corresponding antibody or partition-specific barcode molecule conjugated to a bead.

FIG. 12B shows an example of molecules (e.g., nucleic acid molecules) that can be derived from a cell (such as RNA molecules) that can be processed to append the cell barcode sequence (e.g., partition-specific barcode sequence) to the molecule (e.g., cDNA molecule).

FIG. 12C shows an example of a partition-specific barcode molecule conjugated to a bead, wherein the partition-specific barcode molecule hybridizes to a mRNA molecule.

FIG. 13 shows an example of a reporter oligonucleotide conjugated to an antigen (e.g., an antibody or an MHC multimer).

FIG. 14 illustrates an example of partition-specific barcode molecules carrying bead.

FIG. 15 depicts a system diagram illustrating an example of an analysis system.

FIG. 16 illustrates an exemplary count matrix that can be obtained from experiments described in Examples 8 and 10.

FIG. 17 illustrates exemplary two distinct populations of single cells in the resulting clustering and visualization analyses, indicating that these populations of cells can recognize distinct (e.g., disjoint) sets of the five antigens used in FIG. 16.

FIG. 18 depicts a flowchart illustrating an example of a process for analyzing and visualizing data associated with cells expressing one or more antigen binding molecules.

FIG. 19 schematically illustrates an example of a microwell array.

FIG. 20 schematically illustrates an example of a workflow for processing nucleic acid molecules.

FIG. 21 shows an example workflow for associating a reporter molecule with an antibody using an antibody-binding protein

FIG. 22 illustrates an exemplary count matrix that can be obtained from experiments described in Example 11.

FIG. 23 illustrates an exemplary count matrix that can be obtained from experiments described in Example 12.

FIG. 24A depicts an exemplary workflow for identifying anti-drug antibodies (ADAs) or drug-reactive antibodies (DRAs).

FIG. 24B depicts an exemplary workflow for identifying a patient specific engineered antibody therapeutic (AbTx).

FIG. 24C depicts an exemplary workflow for characterizing anti-drug antibody (ADA) binding to anti-therapeutic (AbTx).

FIG. 24D depicts an exemplary workflow for characterizing anti-drug antibody (ADA) binding to anti-therapeutic (AbTx).

FIG. 25 illustrates an example of a barcode carrying bead.

### DETAILED DESCRIPTION

A better understanding of drug-reactive antibody repertoire can guide drug development and discovery. Successfully identified and validated drug-reactive antibodies have several important uses, each of which can be addressed by the methods described herein. In one aspect, provided herein is a method enabling the capture of drug-specific antibodies. In some aspects, a method described herein utilizes a reporter oligonucleotide conjugated antigen to profile samples from a subject treated with an antibody-drug conjugate (ADC) and/or an antibody therapeutic (AbTX). In some aspects, a method provided herein enables quantitation of many features of the subject's response to the ADC or AbTX. For example, the number of cells that express drug-reactive antibody can be quantified. In another example, a diversity of anti-drug antibody response in the subject can be quantified. In yet another example, the subject's response to the ADC or AbTX can be monitored. In some aspects, a method provided herein can assess whether or not an engineered or modified version of an existing ADC or AbTX is recognized by a pre-existing immune response in the subject.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives can be used and other changes can be made without departing from the spirit or scope of the subject matter presented here. It will be readily understood that the aspects, as generally described herein, and illustrated in the drawings, can be arranged, substituted, combined, and designed in a wide variety of different configurations, all of which are explicitly contemplated and make part of this application.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combinations are individually and explicitly disclosed herein.

The section headings used herein are for organizational purposes only and are not to be construed as limiting the subject matter described.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “real time,” as used herein, can refer to a response time of less than about 1 second, a tenth of a second, a hundredth of a second, a millisecond, or less. The response time can be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals can include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which can be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together can constitute a human genome.

The terms “adaptor(s)”, “adapter(s)” and “tag(s)” can be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides (e.g., nucleic acid barcode molecules). The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing can be performed using nucleic acid amplification, polymerase chain reaction (PCR) (e.g., digital PCR, quantitative PCR, or real time PCR), or isothermal amplification. Such systems can provide a plurality of raw genetic data corresponding to the genetic information of a subject (e.g., human), as generated by the systems from a sample provided by the subject. In some examples, such systems provide sequencing reads (also “reads” herein). A read can include a string of nucleic acid bases corresponding to a sequence of e.g., a nucleic acid barcode molecule that has been sequenced. In some situations, systems and methods provided herein can be used with proteomic information.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. For example, in the methods and systems described herein, hybridization and reverse transcription of a nucleic acid molecule (e.g., a messenger RNA (mRNA) molecule) of a cell with a nucleic acid barcode molecule (e.g., a nucleic acid barcode molecule containing a barcode sequence and a nucleic acid primer sequence complementary to a nucleic acid sequence of the mRNA molecule) results in a barcoded nucleic acid molecule that has a sequence corresponding to the nucleic acid sequence of the mRNA and the barcode sequence (or a reverse complement thereof). A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the mRNA.

The term “sample,” as used herein, generally refers to a biological sample of a subject. The biological sample can include any number of macromolecules, for example, cellular macromolecules. The sample can be a cell sample. The sample can be a cell line or cell culture sample. The sample can include one or more cells. The sample can include one or more microbes. The biological sample can be a nucleic acid sample or protein sample. The biological sample can also be a carbohydrate sample or a lipid sample. The biological sample can be derived from another sample. The sample can be a tissue sample, such as a biopsy, core biopsy, needle aspirate, or fine needle aspirate. The sample can be a fluid sample, such as a blood sample, urine sample, or saliva sample. The sample can be a skin sample. The sample can be a cheek swab. The sample can be a plasma or serum sample. The sample can be a cell-free or cell free sample. A cell-free sample can include extracellular polynucleotides. Extracellular polynucleotides can be isolated from a bodily sample that can be selected from the group consisting of blood, plasma, serum, urine, saliva, mucosal excretions, sputum, stool and tears.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

The terms “biological particle” and “analyte carrier” are used interchangeably herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from an biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may be or include a primer. The molecular tag may be, or include, a protein. The molecular tag may include a polypeptide. The molecular tag may be a barcode.

### Methods of Characterizing Anti-Drug Antibodies

A drug, such as an antibody or antibody-based drug, can elicit an immune response when administered to a subject. In some such cases, the subject can produce antibodies against the antibody or antibody-based drug (i.e., an antigen binding molecule; anti-drug antibody or drug-reactive antibody) that can bind to the antibody or antibody-based drug (i.e., the antigen), thus inhibiting and/or preventing efficacy of the antibody or antibody-based drug. Such drug-reactive antibodies (DRAs) or anti-drug antibodies (ADAs) can result in decreased effectiveness of the antibody or antibody-based drug and/or side effects occurring as a result of the elicited immune response. In some embodiments, interaction between an antigen and an antigen binding molecule can be beneficial. For example, interaction between an antibody of a subject (i.e., the antigen binding molecule) and an antigen (i.e., a vaccine composition) can indicate that immunity can be conferred to the subject upon administering the antigen to the subject.

Provided herein are methods for characterizing anti-drug antibodies (ADAs) or drug-reactive antibodies (DRAs) via barcoding. The terms “anti-drug antibody (ADA)” and “drug-reactive antibody (DRA)” are used interchangeably herein. In an embodiment, provided herein is a method for identifying an antigen binding molecule that can interact with an antigen conjugated to a reporter oligonucleotide (i.e., a reporter oligonucleotide conjugated antigen), wherein the reporter oligonucleotide includes a reporter barcode. In an embodiment, an antigen binding molecule can be an antibody or antigen binding fragment thereof produced by a subject. In an embodiment, an antigen binding molecule can be a therapeutic antibody or antigen binding fragment thereof. In some embodiments, methods provided herein can be useful for identifying an antibody or antigen binding fragment thereof that can interact with an antigen. For example, an antibody from a subject can be identified that can interact with an antigen that subject has been exposed to.

Methods for identifying an antigen binding molecule that can interact with an antigen can include contacting a composition comprising at least one antigen binding molecule with an antigen conjugated to a reporter oligonucleotide. In some embodiments, a composition can include one or more antibodies or antigen binding fragments thereof. In some embodiments, antibodies or antigen binding fragments thereof can be of a subject (e.g., a human). Examples of antibodies and antigen binding fragments thereof are described elsewhere herein. In some embodiments, a composition can include an immune cell, for example a cultured immune cell or an immune cell of a subject. A composition comprising an immune cell can include, for example, a B cell, natural killer (NK) cell, a T-Reg cell, a CAR-T cell, a lymphocyte, T cell or a combination thereof. In some embodiments, the composition comprising an immune cell includes a B cell, natural killer (NK) cell, a lymphocyte, or a combination thereof. In some embodiments, a composition comprising an immune cell can be a composition obtained from a subject, such as a blood sample, serum sample, leukopheresis sample, or tissue sample. In some embodiments, a composition comprising an immune cell can be derived from a composition obtained from a subject, such as an antibody or antigen binding fragment thereof or an immune cell that is isolated from a sample obtained from a subject. A composition can be of a subject that was previously exposed to or may be exposed to an antigen. For example, a composition can be a sample from a subject that is currently undergoing or is a candidate for a therapy, such as a subject that has received, is receiving, or may receive in the future an antibody therapy. In some embodiments, methods provided herein can identify whether an antigen binding molecule in the subject can bind an antigen.

Methods provided herein can include isolating the antigen binding molecule bound to the antigen at least by using a reporter oligonucleotide. An antigen binding molecule isolated by such a method can have affinity to the antigen. In some embodiments, methods can include identifying the antigen binding molecule. In some embodiments, for example, an identified antigen binding molecule can be an anti-drug antibody (ADA) or a drug-reactive antibody (DRA). In some aspects, a method described herein utilizes an oligonucleotide conjugated antigen (e.g., an antigen conjugated to a reporter oligonucleotide) to profile samples from a subject treated with an antibody-drug conjugate (ADC) and/or an antibody therapeutic (AbTX). In some aspects, a method provided herein enables quantitation of features of the subject's response to the ADC or AbTX. For example, the number of cells that express drug-reactive antibody can be quantified. In another example, a diversity of anti-drug antibody response in the subject can be quantified. In yet another example, the subject's response to the ADC or AbTX can be monitored. In some aspects, a method provided herein can assess whether or not an engineered or modified version of an existing ADC or AbTX is recognized by a pre-existing immune response in the subject.

A candidate AbTX or ADC can be converted into an antigen by conjugated a reporter oligonucleotide to the constant region or to a specific site of the AbTX or ADC via a number of methods described herein. This can then be used to stain a sample containing B cells from a subject or a naive donor or engineered cells expressing a known drug-reactive antibody. Using barcoding as described herein, the binding of an antigen conjugated to a reporter oligonucleotide to a memory B cell or a plasma cell or a plasmablast can be detected.

The ADAs isolated using the methods described herein can be used to validate the efficacy of AbTX and ADC re-engineering. By expressing and labeling these ADAs with reporter oligonucleotides and staining engineered cells expressing mutants of the AbTX or ADC of choice, the same workflow described herein can be used to confirm that the new AbTX/ADC variant is not bound by the same antibodies. Furthermore, use of these antibodies in functional assays such as antibody-dependent cellular cytotoxicity (ADCC), antibody-dependent cellular phagocytosis (ADCP), antibody-dependent complement deposition (ADCD), and other assays can be used to identify ADAs with therapeutic value—such ADAs can be developed as drugs to be administered to subjects experiencing side effects induced by an AbTX or ADC. Such ADAs can also be deployed as reference standards in traditional serum-based ADA assays. This is especially important for AbTX and ADCs that have been engineered to be recycled efficiently by addition of Fc domains that can be recognized by the neonatal Fc receptor (FcRn, product of the FCGRT gene). The methods provided herein can also be more broadly used to characterize the response in subjects with diverse immunoglobulin haplotypes, and in combination with traditional statistical models such as contingency tests, GWAS, PheWAS, and cellular assays discover genomic and other elements associated with the development of ADAs.

In some embodiments, a method provided herein can quantify a subject's response to an antigen. For example, the method can include (a) contacting (i) a composition comprising one or more cells from the subject wherein the one or more cells express at least one antigen binding molecule with (ii) a reporter oligonucleotide conjugated antigen, wherein the reporter oligonucleotide comprises a reporter barcode and wherein the antigen is an antibody therapeutic; (b) measuring the number of cells that express the at least one antigen binding molecule that bind to the antibody therapeutic.

In some embodiments, a method provided herein can determine diversity of a subject's immune response to an antigen. For example, the method can include (a) contacting (i) a composition comprising one or more cells from the subject wherein the one or more cells express one or more antigen binding molecules with (ii) a reporter oligonucleotide conjugated antigen, wherein the reporter oligonucleotide comprises a reporter barcode and wherein the antigen is an antibody therapeutic; (b) sequencing the one or more antigen binding molecules; and (c) identifying the one or more antigen binding molecules that bind to the antibody therapeutic to determine the diversity of a subject's immune response to the antibody therapeutic. In some embodiments, the method can further include (d) isolating the one or more antigen binding molecules. In some embodiments, the diversity of a subject's immune response is production of different antibodies and/or antibody lineages.

In some embodiments, a method provided herein can monitor a subject's response to an antibody therapeutic or antibody drug conjugate (ADC). For example, the method can include (a) contacting a composition comprising one or more cells from the subject wherein the one or more cells express one or more antigen binding molecules with (ii) a reporter oligonucleotide conjugated antigen, wherein the reporter oligonucleotide comprises a reporter barcode; (b) identifying the one or more antigen binding molecules; and (c) measuring the number of cells that express at least one antigen binding molecule that bind to the antibody therapeutic or ADC over a course of time. In some embodiments, the method can further include (d) isolating the one or more antigen binding molecule. In some embodiments, the course of time is about bi-weekly, about monthly, or about yearly.

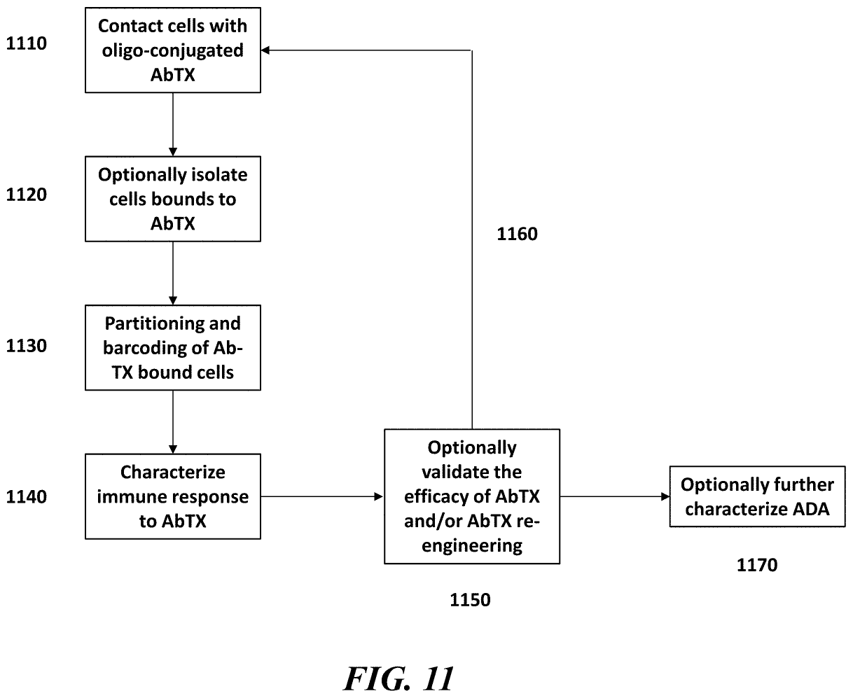

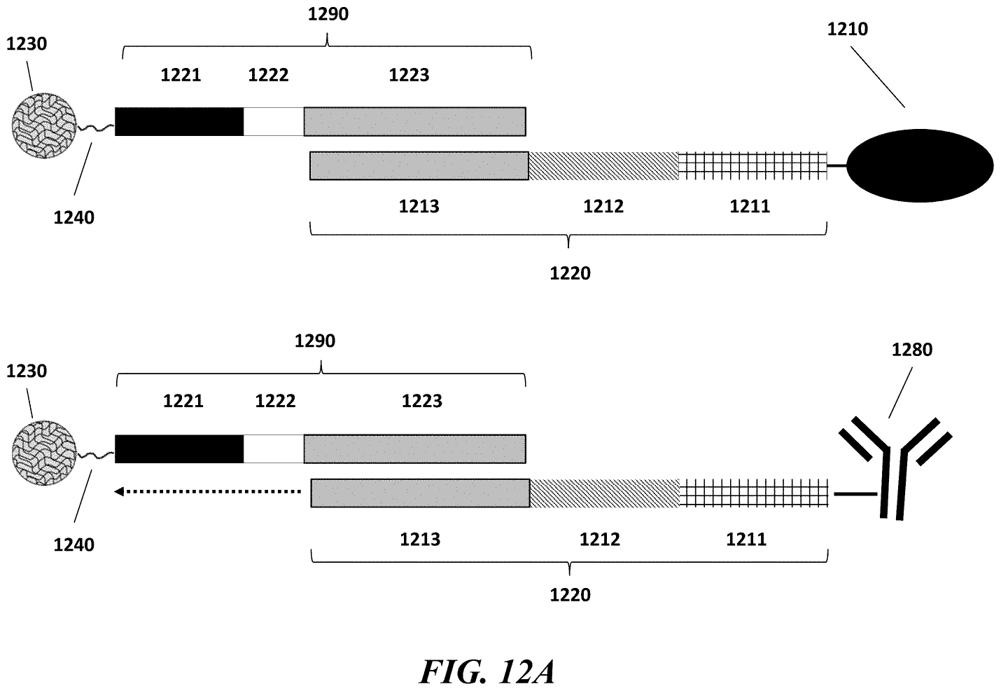

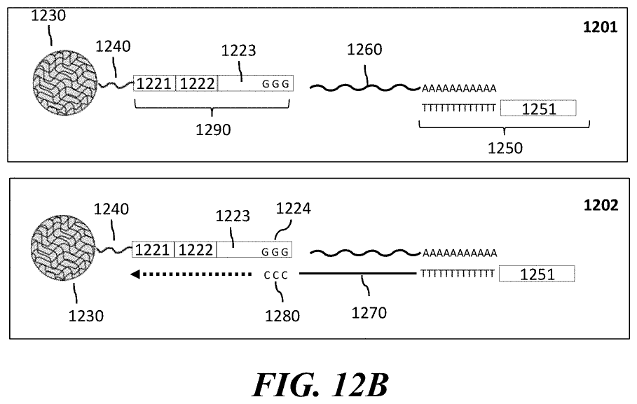

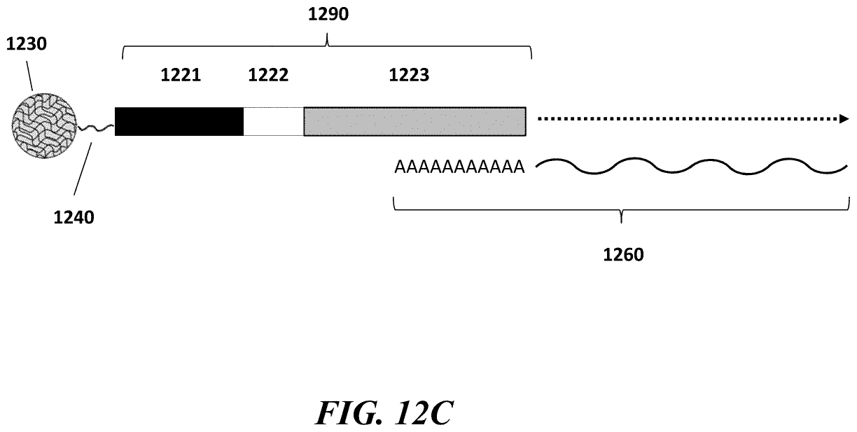

An exemplary workflow is described in FIG. 11. In some embodiments, in operation 1110, cells (such as a sample including B cells) are contacted with an antibody therapeutic (AbTx) complex, e.g., a reporter oligonucleotide conjugated to an antibody therapy (AbTx), such as a therapeutic antibody or an antibody drug conjugate. In some instances, the AbTx complex includes a detectable tag (such as a fluorophore) as described elsewhere herein and cells (e.g., cells comprising a BCR) that bind the AbTx complex are optionally isolated (e.g., using flow cytometry) from cells not bound to the complex in process 1120. Cells bound to the AbTx complex are then partitioned into partitions in operation 1130 with partition-specific barcode molecules, and the reporter oligonucleotide of the AbTx complex and the nucleic acids of the cell can then be barcoded using the partition-specific barcode molecules for subsequent processing and analysis (such as by nucleic acid sequencing) in operation 1140. In some instances, operation 1130 includes partitioning a plurality of cells and a plurality of barcoded beads (e.g., beads conjugated to partition-specific barcode molecules) as described elsewhere herein to provide a plurality of partitions (e.g., a plurality of droplets or a plurality of wells in a micro/nanowell array) containing at most a single cell and a single barcoded bead. In some instances, operation 1130 includes generating barcoded nucleic acid molecules, wherein a common barcode sequence (e.g., a partition-specific barcode sequence) is appended to the AbTx reporter oligonucleotide and nucleic acids derived from the cell, such as BCR sequences from a B cell (see, e.g., FIGS. 12A-12C for a representative barcoding scheme). Thus, in operation 1140, a common barcode (e.g., a partition-specific barcode) sequence can be utilized to associate a specific AbTx with an immune response, e.g., a specific BCR of a B cell. As such, this allows the identification of putative anti-drug antibodies (ADAs), which can optionally be further analyzed in operations 1150, 1160, and/or 1170. For example, in operation 1150, the putative ADAs identified in operation 1140 can themselves be used to validate the efficacy of an AbTx (e.g., therapeutic antibody or antibody drug conjugate) and/or be utilized to validate the efficacy of an AbTx upon re-engineering (e.g., operations 1110-1140 are repeated in operation 1160 with one or more re-engineered AbTx with the goal of observing an altered or reduced ADA response). For example, in some instances, operation 1150 can include expressing one or more of the putative ADAs identified in operation 1140 and labeling these ADAs with oligonucleotides, e.g., reporter oligonucleotides, as described elsewhere herein. Mutants or alterations of the AbTx of interest can be provided, and operations 1110-1140 described above can be used to confirm that the new AbTx variant is not bound by the ADAs. In some embodiments, the ADAs identified in operation 1140 can be further characterized in operation 1170. For instance, use of these antibodies in functional assays such as ADCC, ADCP, ADCD, and other assays can be used to identify ADAs with therapeutic value—such ADAs can be developed as drugs to be administered to subjects experiencing side effects induced by an AbTx or ADC by neutralizing the drug or promoting its destruction. Such ADAs can also be deployed as reference standards in traditional serum-based ADA assays. In some instances, this can be especially important for AbTx that have been engineered to be recycled efficiently by addition of Fc domains that can be recognized by the neonatal Fc receptor (FcRn, product of the FCGRT gene). Molecules identified in operation 1140 more broadly can also be used to characterize the response in patients with diverse immunoglobulin haplotypes, and in combination with traditional statistical models such as contingency tests, GWAS, PheWAS, and cellular assays discover genomic and other elements associated with the development of ADAs.

Labeling Antigens with Barcodes

In the methods and systems described herein, one or more antigens (e.g., an antigen that is an antibody therapeutic or antibody drug conjugate or antibody therapeutic or antibody drug conjugate bound to its antigenic target (AgTx)) capable of binding to or otherwise coupling to one or more cell features or antibodies can be used to characterize cells and/or cell features. In some instances, cell features can include cell surface features. Cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof.

The term “barcode,” as used herein, generally refers to a label, or identifier, that conveys or is capable of conveying information about an analyte. A barcode can be part of an analyte. A barcode can be independent of an analyte. A barcode can be attached to an analyte (e.g., a reporter oligonucleotide). A barcode can be a combination of identifiers in addition to an endogenous characteristic of the analyte (e.g., size of the analyte or end sequence(s)). A barcode can be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The antigen can contain (e.g., is attached to) a reporter oligonucleotide that is indicative of the cell surface feature to which the binding group binds. For example, the reporter oligonucleotide can contain a reporter barcode that permits identification of the labeling agent. For example, an antigen that is specific to one type of cell feature (e.g., a first cell surface feature) can have coupled thereto a first reporter oligonucleotide, while an antigen that is specific to a different cell feature (e.g., a second cell surface feature) can have a different reporter oligonucleotide coupled thereto. For a description of exemplary methods of labeling antigens with reporter oligonucleotides containing reporter barcodes, and methods of use, see, e.g., U.S. Pat. No. 10,550,429; U.S. Pat. Pub. 20190177800; and U.S. Pat. Pub. 20190367969, which are each incorporated by reference herein in their entirety.

In a particular example, a library of potential cell feature labeling antigens can be provided associated with reporter oligonucleotides, e.g., where a different reporter oligonucleotide sequence is associated with each labeling antigen capable of binding to a specific cell feature. In some aspects, different members of the library can be characterized by the presence of a different reporter oligonucleotide sequence label, e.g., an antibody capable of binding to a first type of protein may have associated with it a first known oligonucleotide sequence, while an antibody capable of binding to a second protein (i.e., different than the first protein) may have a second known oligonucleotide sequence associated with it.

The cells can be incubated with the library of antigens conjugated with reporter oligonucleotides that can represent antigens to a broad panel of different cell features, e.g., receptors, proteins, etc., and which include their associated reporter oligonucleotides. Unbound antigens can be washed from the cells. The cells can then be co-partitioned (e.g., into droplets or wells) along with partition-specific barcode molecules (e.g., attached to a bead, such as a gel bead). As a result, the partitions can include the cell or cells, as well as the bound antigens and their known, associated reporter oligonucleotides with reporter barcodes and partition-specific barcode molecules with partition-specific barcodes.

In other instances, e.g., to facilitate sample multiplexing, an antigen that is specific to a particular cell feature can have a first plurality of the antigen (e.g., an antibody or lipophilic moiety) coupled to a first reporter oligonucleotide with a first reporter barcode and a second plurality of the antigen coupled to a second reporter oligonucleotide with a second reporter barcode. In this way, different samples or groups can be independently processed and subsequently combined together for pooled analysis (e.g., partition-based barcoding as described elsewhere herein). See, e.g., U.S. Pat. Pub. 20190323088, which is hereby incorporated by reference its entirety.

In some aspects, these reporter oligonucleotides with reporter barcodes can contain nucleic acid barcode sequences that permit identification of the antigen which the reporter oligonucleotide is coupled to. The selection of oligonucleotides with barcodes as a reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the oligonucleotides to the antigens can be achieved through any of a variety of direct or indirect, covalent or non-covalent associations or attachments. For example, oligonucleotides can be covalently attached to a portion of an antigen (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to antigens. Commercially available kits, such as those from Thunderlink and Abcam, and techniques common in the art can be used to couple reporter oligonucleotides with reporter barcodes to antigens as appropriate. In some example, an antigen is indirectly (e.g., via hybridization) coupled to a partition-specific barcode molecule. For instance, the antigen can be directly coupled (e.g., covalently bound) to a reporter oligonucleotide that comprises a sequence that hybridizes with a sequence of the partition-specific barcode molecules. Hybridization of the partition-specific barcode molecules to the reporter oligonucleotide couples the antigen to the partition-specific barcode molecules. In some embodiments, the reporter oligonucleotides are releasable from the antigen, such as upon application of a stimulus. For example, the reporter oligonucleotide can be attached to the antigen through a labile bond (e.g., chemically labile, photolabile, thermally labile, etc.) as generally described for releasing molecules from supports elsewhere herein. In some instances, the reporter oligonucleotides and/or the partition-specific barcode molecules described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the antigen can include a reporter oligonucleotide with a reporter barcode and a tag. A tag can be an enzyme, a fluorophore, a quantum dot, a covalently or non-covalently attached protein or peptide, a carbohydrate, a radioisotope, a molecule capable of a colorimetric reaction, a magnetic particle, a small molecule, or any other suitable molecule or compound capable of detection. The tag can be conjugated to a labeling agent (or a reporter oligonucleotide with a reporter barcode) either directly or indirectly (e.g., the tag can be conjugated to a molecule that can bind to the labeling agent or the reporter oligonucleotide).

In some cases, a reporter oligonucleotide can be attached (e.g., covalently linked such as conjugated, e.g., directly or indirectly via a linker, or non-covalently bound through a binding interaction) to an antibody via an antibody-binding protein. For example, a reporter oligonucleotide and an antibody-binding protein can form a complex. The complex can bind to a respective antibody through the antibody-binding protein.

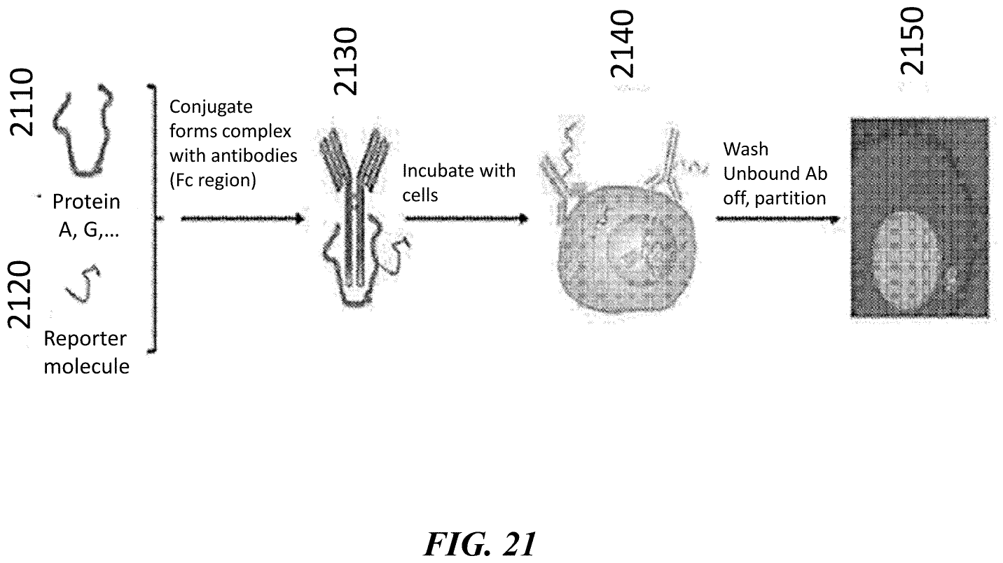

FIG. 21 shows an example workflow for associating a reporter molecule on an antibody using an antibody-binding protein. An antibody binding protein 2110, e.g., Protein A or Protein G, and an a reporter molecule 2120 are conjugated to the Fc region of an antibody, forming a complex 2130 comprising the antibody, the antibody-binding protein 2110, and the reporter molecule 2120. The complex 2130 is incubated with cells and unbound antibody is washed out. When the complex 2130 binds to a cell, the complex and the cell 2140 are partitioned into a droplet 2150 for further analysis, the methods and systems of which are described herein below.

An antibody-binding protein can have fast adsorption kinetics, slow desorption kinetics, and/or a low binding equilibrium constant. Any methods for adding chemical functionality to peptides or protein can be used. Some methods can include attaching a reporter molecule to specific amino acids or chemical groups (e.g., chemical groups present in multiple types of protein) on the antibody-binding protein. The conjugation of antibody-binding proteins and reporter molecules can be performed using any known methods for forming antibody-nucleic acid conjugation, e.g., using click chemistry. Dissociation of the antibody-binding protein/reporter molecule complexes can be prevented by crosslinking (e.g., using a crosslinker such as formaldehyde), protein engineering, or adding the protein-binding proteins in excess.

Examples of antibody-binding proteins can include proteins that bind to the constant (Fc) region of antibodies, such as Protein A, Protein G, protein L, or fragments thereof. Other binding proteins (e.g., streptavidin) can be expressed as fusion proteins with antibody-binding proteins, and used to associate reporter molecules (e.g., by binding of biotinylated reporter molecules to a streptavidin-protein A fusion protein). Other antibody-binding proteins or domains can provide additional binding affinity for various antibody classes. In some cases, the antibody-binding protein can be an antibody, e.g., a secondary antibody for the antibody targeting the sample. The secondary antibody can include a reporter molecule described herein, e.g., an oligonucleotide with a reporter barcode and a poly-A or poly-T terminated sequence.

The antibody-binding proteins can be engineered to introduce additional functionalities. Antibody-binding proteins can be engineered to contain amino acids with functional groups amenable to conjugation with oligonucleotides (e.g., reporter molecules). For example, the antibody-binding proteins can naturally have or be engineered to have cysteine residues, e.g., for controlling stoichiometry and/or attachment location of the reporter molecules. The antibody-binding proteins can be engineered to have non-natural amino acid residues, e.g., for targeted crosslinking of binding proteins and antibodies. The antibody-binding proteins can be engineered to have tags, e.g., fluorescent tags (e.g., by fusing with a fluorescent protein such as green fluorescent protein (GFP), red fluorescence protein (RFP), yellow fluorescent protein (YFP)), and/or affinity tags for purification and visualization. The fluorescent tags and/or the affinity tags can be cleavable. In some cases, the antibody-binding protein can be engineered to have one or more barcode attachment sites per protein.

Also provided herein are kits comprising antibody-binding proteins conjugated with reporter molecules in e.g., well plates. Antibody for an assay can be incubated with the antibody-binding proteins conjugated with reporter molecules at a specified concentration without interfering with the antibody's binding site and/or without the need for any chemistry to be carried out in one's hands to conjugate the reporter molecule to the antibody.

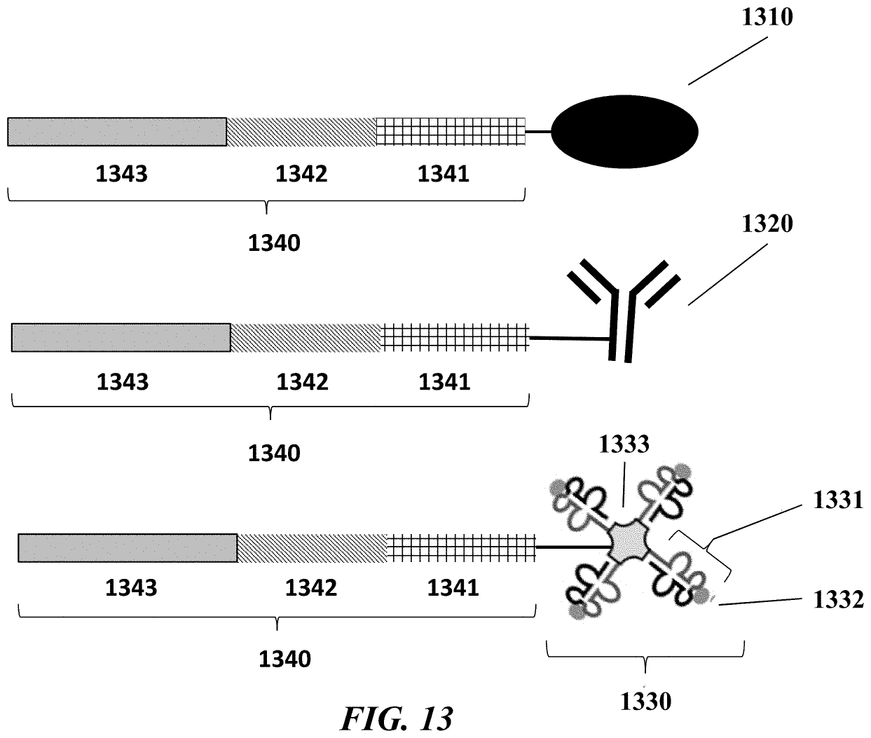

FIG. 13 describes exemplary antigens (1310, 1320, 1330) conjugated to a reporter oligonucleotide (1340) attached thereto. The antigen 1310, 1320, or 1330 is attached (either directly, e.g., covalently attached, or indirectly) to a reporter oligonucleotide 1340. A reporter oligonucleotide 1340 can contain a reporter barcode sequence 1342 that identifies the antigen 1310, 1320, or 1330. A reporter oligonucleotide 1340 can also contain one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 13, in some instances, reporter oligonucleotide 1340 conjugated to an antigen (e.g., 1310, 1320, 1330) can include a functional sequence 1341 (e.g., an adaptor), a barcode sequence that identifies the antigen (e.g., 1310, 1320, 1330), and functional sequence (e.g., adaptor) 1343. Capture handle 1343 can be configured to hybridize to a complementary sequence (e.g., a capture sequence), such as a complementary sequence (e.g., capture sequence) present on a partition-specific barcode molecules (not shown), such as those described elsewhere herein. A capture handle can include a sequence that is complementary to a capture sequence on a partition-specific barcode molecule. In some instances, a partition-specific barcode molecule is attached to a support (e.g., a bead, such as a gel bead), such as those described elsewhere herein. For example, partition-specific barcode molecules can be attached to the support via a releasable linkage (e.g., comprising a labile bond), such as those described elsewhere herein. In some instances, a reporter oligonucleotide 1340 includes one or more additional functional sequences, such as those described above.

In some instances, antigen 1310 is a protein or polypeptide (e.g., an antigen or prospective antigen) conjugated to reporter oligonucleotide 1340. Reporter oligonucleotide 1340 contains reporter barcode sequence 1342 that identifies protein or polypeptide 1310 and can be used to infer the presence of, e.g., a binding partner of protein or polypeptide 1310 (i.e., a molecule or compound to which the protein or polypeptide binds). In some instances, 1310 is a lipophilic moiety (e.g., cholesterol) comprising reporter oligonucleotide 1340, where the lipophilic moiety is selected such that 1310 integrates into a membrane of a cell or nucleus. Reporter oligonucleotide 1340 contains reporter barcode sequence 1342 that identifies lipophilic moiety 1310 which in some instances is used to tag cells (e.g., groups of cells, cell samples, etc.) for multiplex analyses as described elsewhere herein.

In some instances, the antigen is an antibody 1320 (or an epitope binding fragment thereof) including reporter oligonucleotide 1340. Reporter oligonucleotide 1340 includes reporter barcode sequence 1342 that identifies antibody 1320 and can be used to infer the presence of, e.g., a target of antibody 1320 (i.e., a molecule or compound to which antibody 1320 binds).

In other embodiments, antigen 1330 includes an MHC molecule 1331 including peptide 1332 and oligonucleotide 1340 that identifies peptide 1332. In some instances, the MHC molecule is coupled to a support 1333. In some instances, support 1333 is streptavidin (e.g., MHC molecule 1331 can include biotin). In other embodiments, support 1333 is a polysaccharide, such as dextran. In some instances, reporter oligonucleotide 1340 can be directly or indirectly coupled to MHC labeling agent 1330 in any suitable manner, such as to MHC molecule 1331, support 1333, or peptide 1332. In some embodiments, labeling agent 1330 includes a plurality of MHC molecules, i.e., is an MHC multimer, which can be coupled to a support (e.g., 1133). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labeling of various antigens, including antibody and MHC-based labeling agents, reporter oligonucleotides, and methods of use, see, e.g., U.S. Pat. No. 10,550,429 and U.S. Pat. Pub. 20190367969, which are each incorporated by reference herein in their entirety.

### Oligonucleotides

An oligonucleotide can be a molecule which can be a chain of nucleotides. Oligonucleotides described herein can include ribonucleic acids. Oligonucleotides described herein can include deoxyribonucleic acids. In some cases, oligonucleotides can be of any sequence, including a user-specified sequence.

In some embodiments, an oligonucleotide can include G, A, T, U, C, or bases that are capable of base pairing reliably with a complementary nucleotide. 7-deaza-adenine, 7-deaza-guanine, adenine, guanine, cytosine, thymine, uracil, 2-deaza-2-thio-guanosine, 2-thio-7-deaza-guanosine, 2-thio-adenine, 2-thio-7-deaza-adenine, isoguanine, 7-deaza-guanine, 5,6-dihydrouridine, 5,6-dihydrothymine, xanthine, 7-deaza-xanthine, hypoxanthine, 7-deaza-xanthine, 2,6 diamino-7-deaza purine, 5-methyl-cytosine, 5-propynyl-uridine, 5-propynyl-cytidine, 2-thio-thymine or 2-thio-uridine are examples of such bases, although many others are known. An oligonucleotide can include an LNA, a PNA, a UNA, or an morpholino oligomer, for example. The oligonucleotides used herein can contain natural or non-natural nucleotides or linkages.

An oligonucleotide can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides long. In some cases, an oligonucleotide can be between 10-30, between 10-50, between 10-70, between 10-100, between 20-50, between 20-70, between 20-100, between 30-50, between 30-70, between 30-100, between 40-70, between 40-100, between 50-70, between 50-100, between 60-70, between 60-80, between 60-90, or between 60-100 nucleotides in length. In some cases, an oligonucleotide can be no more than 5, no more than 10, no more than 15, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 nucleotides long.

In some cases, an oligonucleotide can be wholly single stranded. In some cases, an oligonucleotide can be partially double stranded. A partially double stranded region can be at the 3′ end of the oligonucleotide, at the 5′ end of the oligonucleotide, or between the 5′ end and 3′ end of the oligonucleotide. In some cases, there can be more than one double stranded region.

In some cases, an oligonucleotide can have a secondary structure. In some cases, an oligonucleotide can have a tertiary structure. Some oligonucleotides can have a structure such that it can fold on itself (e.g. if one region of the oligonucleotide is complementary to another region of the oligonucleotide) to produce one or more double stranded regions comprising a single strand.

In some cases, a segment of an oligonucleotide able to bind a circular nucleic acid primer can be exposed in a single stranded region of the oligonucleotide or an unfolded region of the oligonucleotide. In some cases, a segment of an oligonucleotide able to bind a circular nucleic acid primer can be in a double stranded or folded region of the oligonucleotide, such that upon melting of the oligonucleotide, such a circular nucleic acid primer can bind.

An oligonucleotide can include a sequence that can be used to isolate or identify the antigen. For example, an oligonucleotide can include a barcode sequence (e.g., a reporter barcode and/or a partition-specific barcode). In some embodiments, different reporter oligonucleotides (e.g., reporter oligonucleotides associated with different antigens) can contain different reporter barcode sequences. A reporter barcode sequence or other feature of an reporter oligonucleotide can be configured to be utilized to identify or isolate the reporter oligonucleotide and/or an antigen associated with the reporter oligonucleotide. For example, identification or isolation can be achieved through base pairing, amplification, sequencing, library creation, imaging (e.g., fluorescent imaging of a label conjugated to the oligonucleotide, for example via the barcode), or other methods.

An oligonucleotide can include any nucleic acid based feature provided herein.

In some cases, the nucleic acid molecule (e.g., a partition-specific barcode molecules) can contain one or more functional sequences, for example, for attachment to a sequencing flow cell, such as, for example, a P5 sequence (or a portion thereof) for Illumina® sequencing. In some cases, the nucleic acid molecule or derivative thereof (e.g., partition-specific barcode molecules) can contain one or more additional functional sequences, such as, for example, a P7 sequence (or a portion thereof) for attachment to a sequencing flow cell for Illumina sequencing. In some cases, the nucleic acid molecule (e.g., a partition-specific barcode molecule) can contain a partition-specific barcode sequence. In some cases, the nucleic acid molecule (e.g., a partition-specific barcode molecule) can further include a unique molecular identifier (UMI). In some cases, the nucleic acid molecule (e.g., a partition-specific barcode molecule) can contain an R1 primer sequence for Illumina sequencing. In some cases, the nucleic acid molecule (e.g., a partition-specific barcode molecule) can contain an R2 primer sequence for Illumina sequencing. In some cases, a functional sequence can include a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence can contain a contiguous or continuous portion or segment, but not all, of a full sequence, for example. In some cases, a downstream procedure may extend the partial sequence, or derivative thereof, to achieve a full sequence of the partial sequence, or derivative thereof. Examples of such nucleic acid molecules (e.g., partition-specific barcode molecules) and uses thereof, as can be used with compositions, devices, methods and systems of the present disclosure, are provided in U.S. Patent Pub. Nos. 2014/0378345 and 2015/0376609, each of which is entirely incorporated herein by reference.

### Conjugation

A reporter oligonucleotide can be coupled to an antigen provided herein. Coupling can include a physical or spatial association between the reporter oligonucleotide and the antigen. In some aspects, coupling can include conjugating the antigen to the reporter oligonucleotide.

An antigen (e.g., a therapeutic antibody or antibody drug complex) can be conjugated to a reporter oligonucleotide. The reporter oligonucleotide can be conjugated anywhere along the amino acid chain of the antigen. In some embodiments, the reporter oligonucleotide can be conjugated to the N terminus, the C terminus, or between the N terminus and the C terminus of the antigen.

In some embodiments, the reporter oligonucleotide conjugated to the antigen does not interfere with binding of the antigen to an antigen binding molecule. A reporter oligonucleotide can be conjugated away from a site on the antigen that binds to the antigen binding molecule (e.g., an epitope). In some embodiments, such as when the binding site is unknown, a reporter oligonucleotide can be conjugated to different parts of the antigen on different copies of the antigen.

Either end (e.g., the 3′ end or the 5′ end) of the reporter oligonucleotide can be conjugated to the antigen. In some embodiments, more than one reporter oligonucleotide can be conjugated to the antigen.

Conjugation of a reporter oligonucleotide to an antigen can preserve the tertiary and/or quaternary structure of the antigen. In some embodiments, the structure of the antigen can be completely preserved. In some embodiments, the structure of a binding site (e.g., a site where the antigen can bind to an antigen binding molecule such as an antibody) can be preserved. In some embodiments, the location and/or orientation of surface residues of the antigen can be preserved.

In some embodiments, the link between an antigen and a reporter oligonucleotide can be stable. Stability can be, for example, under physiological conditions (e.g., physiological pH, temperature, etc.), or under conditions of an assay. In some embodiments, such a link can remain stable for at least 1 hour, at least 6 hours, at least 12 hours, at least 1 day, at least 1 week, at least 1 month, at least 1 year, or a range between any two foregoing values.

In some embodiments, the affinity between an antigen and antigen binding molecule can be not compromised by the conjugation of a reporter oligonucleotide to the antigen. In some such embodiments, the presence of the oligonucleotide or the process of conjugating the oligonucleotide to the antigen may not increase or decrease the affinity of the antigen to the antigen binding molecule.

In some embodiments, a reporter oligonucleotide can be conjugated to an antigen directly using any suitable chemical moiety on the antigen. In some embodiments, a reporter oligonucleotide can be conjugated to an antigen enzymatically, e.g., by ligation. In some cases, a reporter oligonucleotide can be linked indirectly to an antigen, for example via a non-covalent interaction such as a biotin/streptavidin interaction or an equivalent thereof, via an aptamer or secondary antibody, or via a protein-protein interaction such as a leucine-zipper tag interaction or the like.

In some embodiments, a reporter oligonucleotide can be conjugated to an antigen using click chemistry, or a similar method. Click chemistry can refer to a class of biocompatible small molecule reactions that can allow the joining of molecules, such as a reporter oligonucleotide and an antigen. A click reaction can be a one pot reaction, and in some cases is not disturbed by water. A click reaction can generate minimal byproducts, non-harmful byproducts, or no byproducts. A click reaction can be driven by a large thermodynamic force. In some cases, a click reaction can be driven quickly and/or irreversibly to a high yield of a single reaction product (e.g., a reporter oligonucleotide conjugated to an antigen), and can have high reaction specificity. Click reactions can include but are not limited to [3+2] cycloadditions, thiol-ene reactions, Diels-Alder reactions, inverse electron demand Diels-Alder reactions, [4+1] cycloadditions, nucleophilic substitutions, carbonyl-chemistry-like formation of ureas, or addition reactions to carbon-carbon double bonds (e.g., dihydroxylation).

In some embodiments, an antigen can be conjugated to a reporter oligonucleotide by a redox activated chemical tagging (ReACT) reaction. A react reaction can be a chemoselective methionine-bioconjugation that can employ redox reactivity. In some embodiments, for example, oxaziridine-based reagents can enable highly selective, rapid, and robust conjugation. Further description of ReACT chemistry can be found, for example, in (Makishma, Akio. Biochemistry for Materials Science. Elsevier, 2019).

In some embodiments, an antigen can be conjugated to a reporter oligonucleotide by a site-specific sortase motif-dependent conjugation. Site-specific sortase motif-dependent conjugation can be a highly specific platform for conjugation that can rely on the specificity of Sortase A for short peptide sequences (e.g., LPXTG AND GGG).

Sortase A can be a transpeptidase that can be adopted for site-specific protein modification. A reaction catalyzed by Sortase A can result in the formation of an amide bond between a C terminal sorting motif (e.g., LPXTG, where X can be any amino acid) and an N terminal oligoglycine. Such a conjugation reaction can proceed by first cleaving the peptide bond between the threonine and glycine residues with the sorting motif of Sortase A. Sortase A can be used to conjugate an oligonucleotide to either an N terminus or a C terminus of an antigen. Sortase A can retain its specificity while accepting a wide range of potential substrates.

In some embodiments, an antigen can be conjugated to a reporter oligonucleotide by a site-specific photo-crosslinking-dependent conjugation. For example, such photo-crosslinking dependent conjugation can utilize unnatural amino acids or chemical crosslinking. Such photo-crosslinking can be mediated or directed by a peptide in some cases. For example, a peptide or other photosensitive molecule on the antigen can form a covalent bond with a molecule on the oligonucleotide upon activation by a specified wavelength of light. In some embodiments, a peptide or other photosensitive molecule on the reporter oligonucleotide can form a covalent bond with a residue on the antigen upon activation by a specified wavelength of light.

In some embodiments, an antigen can be conjugated to a reporter oligonucleotide by site-specific conformation-dependent conjugation (e.g., glycan-dependent Fc conjugation or GlyCLICK). Such conjugation can generate a reporter oligonucleotide conjugated antigen. For example, deglycosylation of the antigen can allow for site specific conjugation using click chemistry techniques. In some embodiment, an antigen can be conjugated to a reporter oligonucleotide by nitrilotriacetate conjugation

An oligonucleotide can be conjugated to a constant region of an antigen. For example, an oligonucleotide can be conjugated to a constant region of a heavy chain or a constant region of a light chain of an antigen that is an antibody or antigen binding fragment thereof.

An oligonucleotide can be conjugated to a variable region of an antigen. For example, an oligonucleotide can be conjugated to a variable region of a heavy chain or a variable region of a light chain of an antigen that is an antibody or antigen binding fragment thereof.

The reporter oligonucleotide conjugated antigen can include one or more detectable tags. For example, in some instances, the reporter oligonucleotide conjugated antigen can include a fluorophore, metal ion, or other detectable tag. The detectable tag can be conjugated to the reporter oligonucleotide, the antigen, or both.

A reporter oligonucleotide conjugated to an antigen (e.g., a therapeutic antibody or antibody-drug conjugate) can include a sequence that identifies the antigen (e.g., a reporter barcode sequence). In some instances, each antigen can be conjugated to a reporter oligonucleotide containing a unique barcode sequence (e.g., a reporter barcode) that identifies the antigen allowing different antigens to be distinguished from one another, e.g., in a multiplexed antigen assay. In addition to the reporter barcode sequence, in some embodiments, the reporter oligonucleotide conjugated to the antigen (e.g., a therapeutic antibody or antibody-drug conjugate) can include additional sequences that facilitate the processing and identification of the reporter barcode sequence (e.g., through nucleic acid sequencing of nucleic acid barcode molecules). For example, the reporter oligonucleotide can contain one or more of: an adapter sequence, a primer or primer binding sequence, a sequencing primer or sequencing primer binding sequence (such as an R1 or partial R1 sequence), a unique molecular identifier (UMI), a polynucleotide sequence (such as a poly-A or poly-C sequence), or a sequence configured to bind to the flow cell of a sequencer (such as a P5 or P7, or partial sequences thereof).

Contacting with Reporter Oligonucleotide Conjugated Antigens

Contacting a composition containing at least one antigen binding molecule with a reporter oligonucleotide conjugated antigen can include contacting the composition with the antigen (such as a therapeutic antibody or antibody-drug conjugate) such that the antigen can interact with the antigen binding molecule. In some embodiments, upon contacting, the antigen can bind to the antigen binding molecule.

In some embodiments, the composition including at least one antigen binding molecule includes a cell. For example, in some instances, the composition including at least one antigen binding molecule is a cell including an immune receptor, such as a B cell including a B cell receptor (BCR).

Contacting can occur in a vessel, such as a well, a tube, a bead, a partition, a microfluidic system, or in another vessel. In some embodiments, contacting can occur on a membrane or a column. In some embodiments, contacting can occur on a surface or support, such as on a slide, a plate, or a dish.

Contacting can include exposing the at least one antigen binding molecule to the antigen under conditions that can allow binding if the antigen binding molecule has affinity for the antigen. In some embodiments, contacting can occur under physiological conditions, or under conditions that approximate physiological conditions.

In some embodiments, contacting can be performed under conditions that do not significantly alter the structure and/or function of the antigen or the antigen binding molecule. In some embodiments, contacting can be performed under conditions that do not significantly alter (e.g., increase or decrease) the affinity of the antigen binding molecule for the antigen. Conditions can include a pH, a temperature, or other conditions.

In some embodiments, contacting can occur in a buffer having a pH. In some embodiments, for example, contacting can occur in a buffer having a pH of about 4.5, about 5.0, about 5.5, about 6.0, about 6.5, about 7.0, about 7.5, about 8.0, about 8.5, about 9.0, or a range between any two foregoing values.

In some embodiments, contacting can occur at a temperature that can allow protein-protein interactions. In some embodiments, contacting can occur at a temperature of about 10° C., about 15° C., about 20° C., about 25° C., about 30° C., about 35° C., about 40° C., about 45° C., about 50° C., or a range between any two foregoing values.

In some embodiments, a method can include contacting the composition comprising at least one antigen binding molecule with a plurality of antigens, wherein the antigens can be conjugated to reporter oligonucleotides. In some such cases, an antigen of the plurality of antigens can be conjugated to a reporter oligonucleotide that is different than a reporter second oligonucleotide that is conjugated to a second antigen. In some embodiments, one or more such antigens can bind one or more antigen binding molecules in the composition. In some embodiments, one antigen can bind an antigen binding molecule in the composition more strongly than a second antigen.

In some embodiments, a method can include contacting the composition including at least one antigen binding molecule with a plurality of variations of an antigen, wherein the variations of antigens can be conjugated to reporter oligonucleotides. In some such cases, a variation of the plurality of variations of an antigen can be conjugated to a reporter oligonucleotide that is different than a second reporter oligonucleotide that is conjugated to a second variation of the antigen. In some embodiments, one or more such variations of an antigen can bind an antigen binding molecule in the composition. In some embodiments, one variation of an antigen can bind an antigen binding molecule more strongly than a second variation of the antigen.

Variations of an antigen can include, for example, an amino acid mutation (e.g., an insertion, deletion, or point mutation) or differences in glycosylation or other modification to an amino acid. In some embodiments, a variation of an antigen can include a non-natural amino acid. In some embodiments, a variation can, for example, be a variation of a therapeutic (e.g., an antibody or antibody-based drug) that is modified. In some embodiments, a modified therapeutic can be modified to reduce recognition of the therapeutic by the immune system of a subject. In some embodiments, a modified therapeutic can be modified to reduce binding of the therapeutic to an antigen binding molecule, such as an antibody or antigen binding fragment thereof.

### Partitioning Barcoded Antigen Binding Molecules

Antigen binding molecules can be analyzed and/or isolated based at least in part on reporter oligonucleotides conjugated to antigens. In some embodiments, such analysis and/or isolation can allow for identification and/or characterization of an antigen binding molecule that has affinity for the antigen.

In some instances, analyzing and/or isolating an antigen binding molecule bound to an antigen (such as a BCR of a B cell or an anti-drug antibody (ADA) bound to a therapeutic antibody or antibody-drug conjugate) can include hybridizing a reporter oligonucleotide of a reporter oligonucleotide conjugated antigen to a partition-specific barcode molecule to isolate or otherwise separate the antigen binding molecule from other materials (e.g., non-antigen bound materials).

Isolating an antigen binding molecule can include capturing the reporter oligonucleotide. In some embodiments, capturing the reporter oligonucleotide can include annealing the reporter oligonucleotide to a partition-specific barcode molecule. In some embodiments, a partition-specific barcode molecule can include one or more of a partition-specific barcode, a unique molecular identifier, or a template switching oligonucleotide as described elsewhere herein.

In some embodiments, isolating an antigen binding molecule can include pulling down at least one antigen binding molecule using its unique sequence. In some embodiments, the at least one antigen binding molecule can be pulled down without using the partition-specific barcode molecule. In some embodiments, isolating an antigen binding molecule can include pulling down the at least one antigen binding molecule using the partition-specific barcode molecule. In some embodiments, the partition-specific barcode molecule can be attached to a bead, such as those described elsewhere herein. In some embodiments, the partition-specific barcode molecule can be affixed to a slide, affixed to a well, affixed to a tube, or conjugated to a magnetic molecule.

In some embodiments, an antigen binding molecule can be isolated using a pull-down assay. For example, the oligonucleotide can be hybridized to a partition-specific barcode molecule that is conjugated to a protein or a magnetic particle. Upon hybridization, the protein or magnetic particle can be used to separate the antigen binding molecule from other components of the composition, for example by contacting the protein or magnetic particle with a binding partner to the protein or a magnetic field, respectively, and washing away other components of the composition. In some embodiments, for example when an antigen binding molecule is on a cell surface, the cell can be lysed prior to performing such a pull-down assay.

In some embodiments, for example when an antigen binding molecule is on a cell surface, the cell can be lysed or the binding molecule can otherwise be removed from the cell. Such lysis or removal can occur before or after the antigen binding molecule is isolated using a partition. In some embodiments, an antigen binding molecule can be isolated from a composition with the cell (i.e., the cell can remain intact). In some embodiments, an antigen binding molecule can be isolated using a flow cytometry technique.

In some embodiments, an antigen binding molecule can be isolated using a partition. In some cases, the antigen binding molecule bound to the reporter oligonucleotide conjugated antigen can be separated into a partition, for example a bead or another partition provided herein (e.g., droplet or well-based partitioning systems). A partition can include a partition-specific barcode molecule, which can be configured to hybridize to a reporter oligonucleotide conjugated to the antigen.

In some instances, analysis of one or more antigen binding molecules (e.g., using the oligonucleotide labeled antigens described herein) includes a workflow as generally depicted in FIGS. 12A-12C. For example, in some embodiments, cells are contacted with one or more reporter oligonucleotide 1220 conjugated antigens 1210 (e.g., polypeptide, antibody (1280), or pMHC molecule or complex) and optionally further processed prior to barcoding. Optional processing steps can include one or more washing and/or cell sorting steps. In some instances, a cell bound to antigen 1210 (e.g., polypeptide, antibody (1280), or pMHC molecule or complex) conjugated to reporter oligonucleotide 1220 and support 1230 (e.g., a bead, such as a gel bead) conjugated to partition-specific barcode molecules 1290 are partitioned into a partition amongst a plurality of partitions (e.g., a droplet of a droplet emulsion or a well of a micro/nanowell array). In some instances, the partition includes at most a single cell bound to labeling agent 1210. In some embodiments, partition-specific barcode molecules 1290 is attached to support 1230 via a releasable linkage 1240 (e.g., comprising a labile bond) as described elsewhere herein.

With continued reference to FIG. 12A, in some instances, oligonucleotide 1220 conjugated to antigen 1210 (e.g., polypeptide, an antibody (1280), pMHC molecule such as an MHC multimer, etc.) includes a functional sequence 1211 (e.g., an adaptor sequence), a reporter barcode sequence 1212 that identifies the antigen 1210 (e.g., the polypeptide, antibody (1280), or peptide of a pMHC molecule or complex), and capture handle 1213. Capture handle 1213 can be configured to hybridize to a capture sequence, such as capture sequence 1223 present on partition-specific barcode molecule 1290. A capture handle can include a sequence that is complementary to a capture sequence on a partition-specific barcode molecule. In some instances, partition-specific barcode molecule 1290 is attached to a support 1230 (e.g., a bead, such as a gel bead), such as those described elsewhere herein. For example, partition-specific barcode molecule 1290 can be attached to support 1230 via a releasable linkage 1240 (e.g., comprising a labile bond), such as those described elsewhere herein. A partition-specific barcode molecule 1290 can include a reporter capture sequence 1223, a common barcode (e.g., a partition specific barcode 1222, and a functional sequence 1221. In some instances, reporter oligonucleotide 1220 includes one or more additional functional sequences, such as those described above.

Referring to FIGS. 12B-C, in some instances, nucleic acid molecules (e.g., partition-specific barcode molecules) derived from a cell (such as RNA molecules) can be similarly processed to append the partition-specific barcode sequence 1222 to these molecules or derivatives thereof (e.g., cDNA molecules). For example, referring to FIG. 12B, in some embodiments, primer 1250 includes a sequence complementary to a sequence of RNA molecule 1260 (such as an RNA encoding for a BCR sequence) from a cell. In some instances, primer 1250 includes one or more adapter sequences 1251 that are not complementary to RNA molecule 1260. In some instances, primer 1250 includes a poly-T sequence. In some instances, primer 1250 includes a sequence complementary to a target sequence in an RNA molecule. In some instances, primer 1250 includes a sequence complementary to a region of an immune molecule, such as the constant region of a TCR or BCR sequence. Primer 1250 is hybridized to RNA molecule 1260 and cDNA molecule 1270 is generated in a reverse transcription reaction. In some instances, the reverse transcriptase enzyme is selected such that several non-templated bases 1280 (e.g., a poly-C sequence) are appended to the cDNA. Partition-specific barcode molecule 1290 includes a sequence 1224 complementary to the non-templated bases, and the reverse transcriptase performs a template switching reaction onto partition-specific barcode molecules 1290 to generate a nucleic acid molecule including partition-specific barcode 1222 (or a reverse complement thereof) and a sequence of cDNA 1270 (or a portion thereof). In another example, referring to FIG. 12C, in some embodiments, partition-specific barcode molecule 1290 includes capture sequence 1223 complementary to a sequence of RNA molecule 1260 from a cell. In some instances, capture sequence 1223 includes a sequence specific for an RNA molecule. In some instances, capture sequence 1223 includes a poly-T sequence. In some instances, capture sequence 1223 includes a sequence specific for an RNA molecule. In some instances, capture sequence 1223 includes a sequence complementary to a region of an immune molecule, such as the constant region of a TCR or BCR sequence. Capture sequence 1223 can be hybridized to RNA molecule 1260 and a cDNA molecule 1270 (FIG. 12B) is generated in a reverse transcription reaction, generating a nucleic acid molecule including partition-specific barcode sequence 1222 (or a reverse complement thereof) and a sequence of cDNA 1270 (FIG. 12B) (or a portion thereof). The nucleic acid molecules (e.g., nucleic acid barcode molecules) including partition-specific barcode 1222 can then be optionally processed as described elsewhere herein, e.g., to amplify the molecules and/or append sequencing platform specific sequences to the fragments. See, e.g., U.S. Pat. Pub. 20180105808, which is hereby incorporated by reference in its entirety. The nucleic acid barcode molecules, or derivatives generated therefrom, can then be sequenced on a suitable sequencing platform.

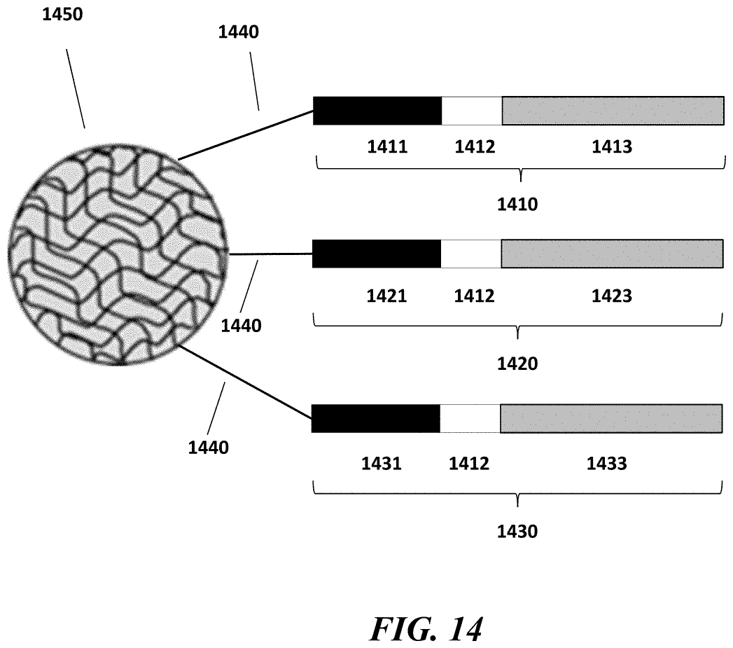

Exemplary partition-specific barcode molecules attached to a support (e.g., a bead) is shown in FIG. 14. In some embodiments, partition-specific barcode molecule 1410 can include functional sequence 1411, partition-specific barcode sequence 1412 and capture sequence 1413. Partition-specific barcode molecule 1420 can include functional sequence 1421, partition-specific barcode sequence 1412, and capture sequence 1423, wherein capture sequence 1423 includes a different sequence than capture sequence 1413. In some instances, functional sequence 1411 and functional sequence 1421 include the same sequence. In some instances, functional sequence 1411 and functional sequence 1421 include different sequences. Although support 1450 is shown including partition-specific barcode molecule 1410 and 1420, any suitable number of partition-specific barcode molecules including partition-specific barcode sequence 1412 are contemplated herein. For example, in some embodiments, support 1450 further includes partition-specific barcode molecule 1430. Partition-specific barcode molecule 1430 can include functional sequence 1431, partition-specific barcode sequence 1412 and capture sequence 1433, wherein capture sequence 1433 includes a different sequence than capture sequence 1413 and 1423. In some instances, partition-specific barcode molecules (e.g., 1410, 1420, 1430) include one or more additional functional sequences, such as a UMI or other sequences described herein.

### Identifying Antigen Binding Molecules

Identifying an antigen binding molecule can include determining the identity of the antigen binding molecule. In some embodiments, an antigen binding molecule can be determined by comparing the antigen binding molecule to a library of antigen binding molecules, by sequencing the antigen binding molecule, using imaging, using histochemical techniques, using spectrometry, or by another technique. In some embodiments, an antigen binding molecule can be identified using a barcode associated with the reporter oligonucleotide (e.g., a reporter barcode on the oligonucleotide conjugated to the antigen, or a partition-specific barcode appended to a partition-specific barcode molecule complementary to the reporter oligonucleotide conjugated to the antigen).

In some embodiments, the sequence (e.g., the amino acid sequence) of the antigen binding molecule can be determined. In some embodiments, the sequence of the entire antibody can be determined. In some embodiments, a portion of the sequence of the antibody, such as the variable region of the light chain or a fragment thereof, the variable region of the heavy chain or a fragment thereof, the constant region of the light chain or a fragment thereof, the constant region of the heavy chain or a fragment thereof, or a combination thereof can be determined. This can be accomplished, for example, by using mass spectrometry or by Edman degradation using a protein sequenator (sequencer).

In some embodiments, an isotype of the antigen binding molecule (e.g., IgA, IgD, IgG, IgE, or IgM) can be determined. Isotype can be determined, for example, by sequencing or by an assay designed to determine the isotype (e.g., an antibody based assay or other assay).

In some embodiments, the antigen binding molecule can be identified based on its affinity to an epitope of the antigen. In some such cases, the sequence of the antigen can be known, and techniques such as epitope mapping techniques can be implemented to identify the antigen binding molecule.

In some embodiments, more than one antigen binding molecules can be identified. For example, a sample from a subject can include more than one antigen binding molecule that can bind to an antigen (e.g., a therapeutic antibody) that can be identified by methods described herein. In some such cases, a first identified antigen binding molecule can have a higher affinity for the antigen than a second identified antigen binding molecule. The more than one antigen binding molecule can bind to the same epitope on the antigen or to different epitopes on the antigen. In some embodiments, 2, 3, 4, 5, 10, 50, 100, or more antibodies can be identified.

### Identification of Mutations in Therapeutic Antibodies or Antibody-Drug Conjugates

One or more mutations in therapeutic antibodies or antibody-drug conjugates that eliminate the recognition of the mutated therapeutic antibodies or antibody-drug conjugates comprising such mutations by the preexisting anti-drug antibodies can be determined by methods provided herein.

A count matrix of cells and drug-reactive antibodies conjugated to reporter oligonucleotides can be derived. In some embodiments, a count matrix of cells expressing drug-reactive antibodies, and therapeutic antibodies conjugated to reporter oligonucleotides can be derived. In some embodiments, a count matrix of cells expressing unmodified or mutated therapeutic antibodies and drug-reactive antibodies conjugated to reporter oligonucleotides can be derived. In some embodiments, a count matrix of cells expressing drug-reactive antibodies, therapeutic antibodies conjugated to reporter oligonucleotides, and therapeutic ligands conjugated to reporter oligonucleotides can be derived. In some embodiments, a count matrix of cells expressing mutated therapeutic antibodies, drug-reactive antibodies conjugated to reporter oligonucleotides, and therapeutic ligands conjugated to reporter oligonucleotides can be derived. The count matrix can, for example, provide data regarding cells (e.g., T cells or B cells) from a composition that containing antibodies that can bind to a therapeutic antibody, antibody-drug conjugate, control antigen, viral antigen, or other etiologic antigen, such as a cancer antigen, control antigen, or decoy antigen.

In some embodiments, cells can be filtered to retain cells that have both a detected VDJ/antibody sequence and non-zero drug-reactive antibody counts and can be sequenced to an appropriate depth. In some embodiments, cells can be sequenced at approximately 5,000 to 100,000 reads per cell for both antigen and VDJ libraries. In some embodiments, cells can be sequenced at approximately 5,000 to 20,000 reads per cell for both antigen and VDJ libraries. In some embodiments, cells can be sequenced at approximately 20,000 to 40,000 reads per cell for both antigen and VDJ libraries. In some embodiments, cells can be sequenced at approximately 40,000 to 60,000 reads per cell for both antigen and VDJ libraries. In some embodiments, cells can be sequenced at approximately 60,000 to 80,000 reads per cell for both antigen and VDJ libraries. In some embodiments, cells can be sequenced at approximately 80,000 to 100,000 reads per cell for both antigen and VDJ libraries. In some embodiments, saturation can occur rapidly.

The count matrix can be retained from cells that are removed. For each antigen, background concentration and ambient concentration can be calculated (e.g., mean, median, or hypergeometric mean), and appropriate deviation for non-cell-associated (empty barcodes) can be calculated. For a given cell retained by the filter, a logarithm (e.g., natural, base 10, or base 2) or similar transformation (e.g., variance stabilizing transformation, square root, or cubic root) can be performed, a pseudocount (e.g., ranging from 1-30) can be added, and the background can be subtracted. The value can then be scaled (e.g., by division) by the deviation or variance of the given antigen. Further, the value can be modified using a Gaussian mixture model for each antigen on a count vector for each cell, and can include, for example, but not limited to, covariates for sequencing depth or number of detected unique molecular identifiers, cell annotations, or other technical or biological covariates, such as the expression level of the BCR within the cell.

Alternatively, in some embodiments, background and/or ambient concentration can be calculated in empty droplets, and subtracted for each antigen. A pseudocount can be added (e.g., ranging from 1-50) in some embodiments. The center log ratio or other appropriate compositional transformation can then be applied.

In some embodiments, the resulting matrix and cellular populations thereof can be visualized using standard dimensional reduction models, for example PCA, UMAP, t-SNE, or Poincare disks. Further application of spectral clustering, granularity/resolution-based/community detections algorithms, or other algorithms can be used to separate cells into groups of cells with distinct drug reactive antibody profiles.

### Enzyme-Tagged Therapeutic Antibodies or Antibody-Drug Conjugates

Methods provided herein can be used for enzyme-tagged therapeutic antibodies or antibody-drug conjugates, wherein the therapeutic antibodies or antibody-drug conjugates can be tagged for example with a sortase motif (LPXTG). In some embodiments, a therapeutic antibody or antibody-drug conjugate can be bound by a cell expressing an anti-drug antibody and captured. Incubation with a protein tag or peptide tag including an N-terminal glycine and addition of a bacterial SrtA protein or an engineered variant thereof (e.g., P94, D160N, or K196T) can confer a protein tag which can then be recognized by a barcoded antibody with an additional round of staining. For example, the covalently or non-covalently attached protein tag is selected from BCCP (biotin carboxyl carrier protein) tag, glutathione-S-transferase tag, green fluorescent protein tag, halo-tag, SNAP tag, CLIP tag, HUH tag, maltose binding protein tag, Nus tag, thioredoxin tag, Fc tag, and CRDSAT tag. For example, the covalently or non-covalently attached peptide tag is selected from ALFA tag, AviTag, C-tag, calmodulin tag, polyglutamate tag, polyarginine tag, E tag, FLAG tag, HA tag, His tag, Myc tag, NE tag, Rho1D4 tag, S tag, SBP tag, Softag 1, Softag 3, Spot tag, Strep tag, T7 tag, TC tag, Ty tag, V5 tag, VSV tag, Xpress tag, Isopeptag, Spy tag, Snoop tag, DogTag, and SdyTag.

### Screening Anti-Drug Antibodies Using a Cell Bead

In some embodiments, a plasma cell contained within a cell bead gel bead with a matrix capturing human IgG constant regions can be screened by tethering a secreted putative anti-drug antibody to the cell bead. An antigen can then be used to detect anti-drug antibodies on the surface of the cell bead, which can then be isolated to detect an anti-drug antibody sequence. In some embodiments, plasma cells incubated with bi-specific or fusion anti-CD45+anti-Ig constant region antibodies with their own reporter barcodes can be used to capture and/or tether a secreted putative anti-drug antibody to the surface of the cell for further screening.

### Screening Anti-Drug Antibodies Using a Biotin Conjugated Antigen in a Cell Bead

In some embodiments, an antigen can be conjugated (directly or indirectly) to biotin, and a cell bead can be magnetized or coated with a magnetic-sensitive matrix. The magnetized cell bead can be pulled through one or more fluidic streams comprising free streptavidin (e.g., on a microfluidic chip). In some embodiment, such streams can wash away excess antigen while pulling the cell bead gel bead toward an electrode. In some embodiments, a droplet can be used in place of a cell bead.

### Capture of Anti-Drug Antibodies Using a Splint Oligonucleotide

In some embodiments, a splint oligonucleotide can be used to capture and/or detect both an anti-drug antibody RNA transcript and genomic DNA. In some embodiments, RTX polymerase can be used to produce a clonable and uniquely amplifiable anti-drug antibody VDJ sequence that can be cell associated. Such sequences can be amplified after next-generation sequencing and analysis, and can enable rapid production of anti-drug antibodies for research or screening purposes.

### Isolation of Antigen Binding Molecules Using a Series of Droplet Generators and Electrodes

In some embodiments, a series of droplet generators and electrodes can be connected to B cells or putative anti-drug antibody expressing cells can be partitioned and incubated. In some embodiments, incubation can allow expression of the anti-drug antibody. Cells can be passed through a second junction where antigen-expressing cells or antigens are placed into droplets or cell bead gel beads with putative anti-drug antibody expressing cells, or into a chamber where electrodes can be used to merge droplets of the two, co-incubated over time, and then passed through a third microfluidic channel, incubated to allow sufficient time for an enzymatic reaction to occur (e.g., 3-20 hours), and screened.

### Efficacy Validation and Re-Engineering Therapeutic Antibodies and Antibody Drug Conjugates

Anti-drug antibodies isolated using a method provided herein can be used to validate the efficacy of a therapeutic antibody or antibody-drug conjugate re-engineering. By expressing and labeling the anti-drug antibodies with oligonucleotides and staining engineered cells expressing mutants of the therapeutic antibody or antibody drug conjugate of choice, a workflow described herein, e.g., in example 1, can be used to confirm that the new therapeutic antibody or antibody-drug variant is not bound by the same antibodies as the original therapeutic antibody or antibody-drug variant. Furthermore, use of such antibodies in functional assays (e.g., ADCC, ADCP, ADCD, or other assays) can be used to identify anti-drug antibodies with therapeutic value. For example, such anti-drug antibodies can be developed as drugs to be administered to patients experiencing side effects induced by a therapeutic antibody or antibody-drug conjugate by neutralizing the drug or promoting its destruction. Such anti-drug antibodies can also be deployed as reference standards in traditional serum-based anti-drug antibody assays. In some embodiments, this can be effective for therapeutic antibodies or antibody-drug conjugates that have been configured to be recycled efficiently, such as by the addition of one or more Fc domains that can be recognized by a neonatal Fc receptor (FcRn, product of the FCGRT gene). Antigens more broadly can also be used to characterize the response in patients with diverse immunoglobulin haplotypes, and in combination with traditional statistical models such as contingency tests, GWAS, PheWAS, and/or cellular assays discover genomic or other elements associated with the development of anti-drug antibodies.

### Identification of Re-Engineered Antigens Recognized by Antigen Binding Molecules

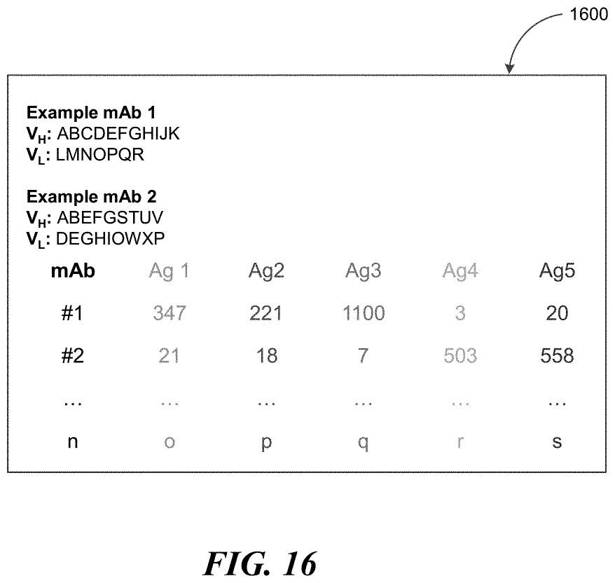

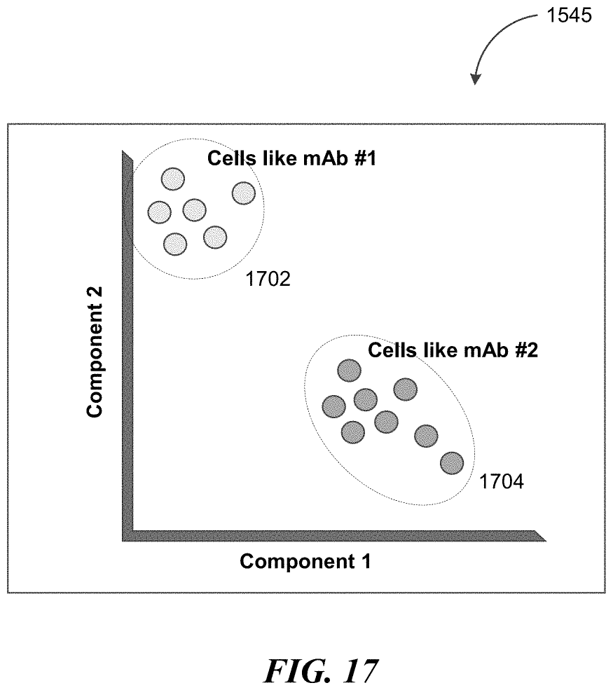

The methods provided herein can be implemented to perform an experiment with 5 different point mutants of adalimumab (FIG. 16). Following the procedure of the method, each point mutant can be labeled with its own unique reporter barcode oligonucleotide population. The presence of 2 distinct populations of single cells in the resulting clustering and visualization analyses can indicate that these populations of cells can recognize distinct (e.g., disjoint) sets of the 5 antigens used (FIG. 17). In some embodiments, mutation or masking of the identified amino acids in the parent antigen (i.e., adalimumab) can ablate recognition of the drug by patient derived anti-drug antibodies, guiding the drug engineering and development process accordingly. In some embodiments, a re-engineered antibody can be knocked into a cell line, expressed, and purified to produce a new antigen (e.g., adalimumab variant) to be used in the same patient samples.

### Analysis of Anti-Drug Antibodies (ADA) or Drug-Reactive Antibodies (DRA)

The methods provided herein can be implemented to isolate and characterize anti-drug antibodies (ADAs) or drug-reactive antibodies (DRAs) via barcoding. For example, an antigen conjugated to a reporter molecule can be used to profile samples from a subject treated with an antibody-drug conjugate (ADC) and/or an antibody therapeutic (AbTx). An exemplary workflow for associating the reporter molecule with an antibody (e.g., an ADA, an ADC, or an AbTx) is shown in FIG. 21. For example, the reporter molecule 2120 can be conjugated with e.g., an antibody-binding protein (e.g., Protein A or Protein G), and the complex 2130 can be formed by non-covalent association between the antibody binding protein and the ADC and/or the AbTX. The complex 2130 can be incubated with cells. In some embodiments, the cells are isolated from a subject. In alternative embodiments, the cells express an AbTx which can be unmodified or modified. Unbound 2130 is washed off, and 2140 can be partitioned, e.g., into a droplet 2150 for further analysis.

### Identification of Anti-Drug Antibodies

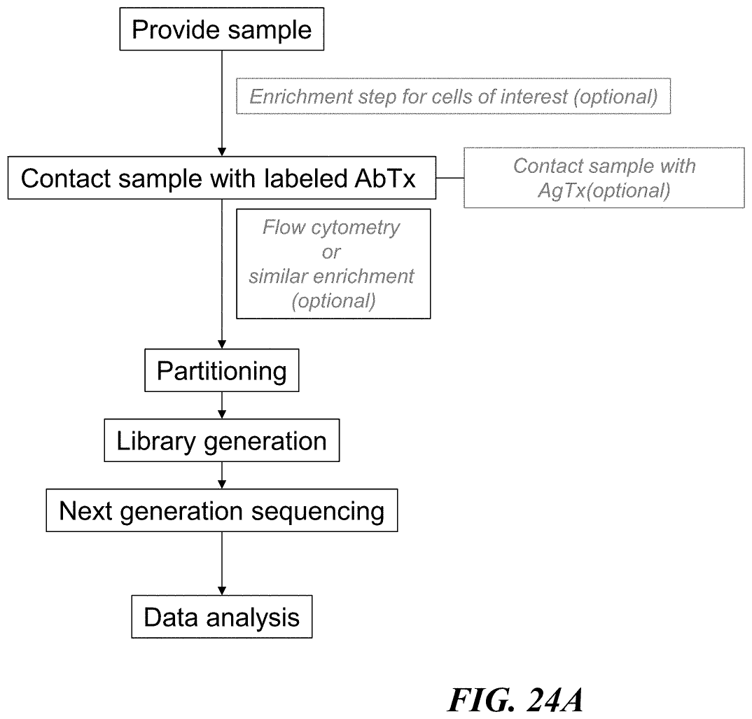

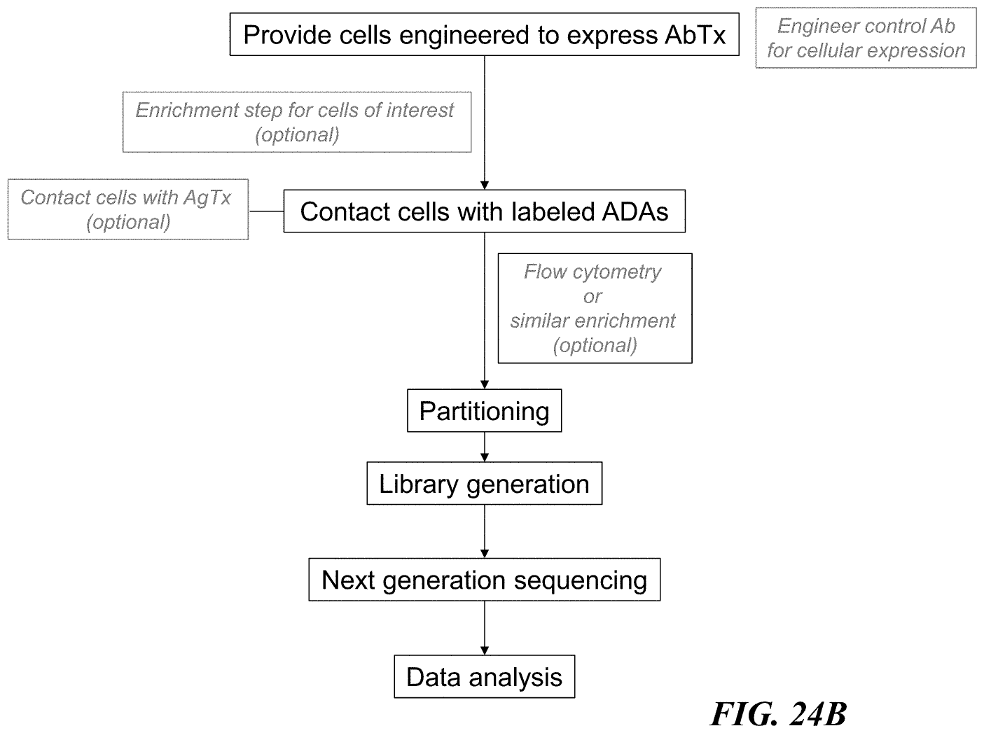

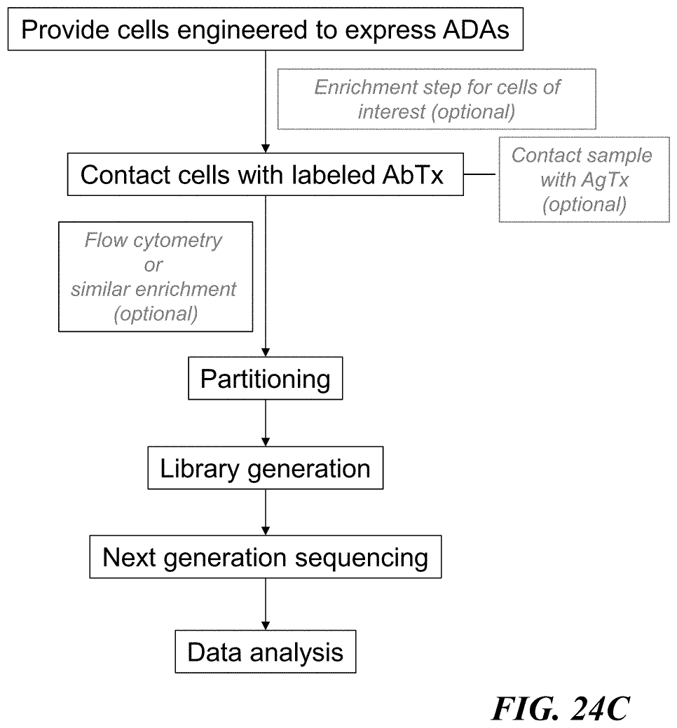

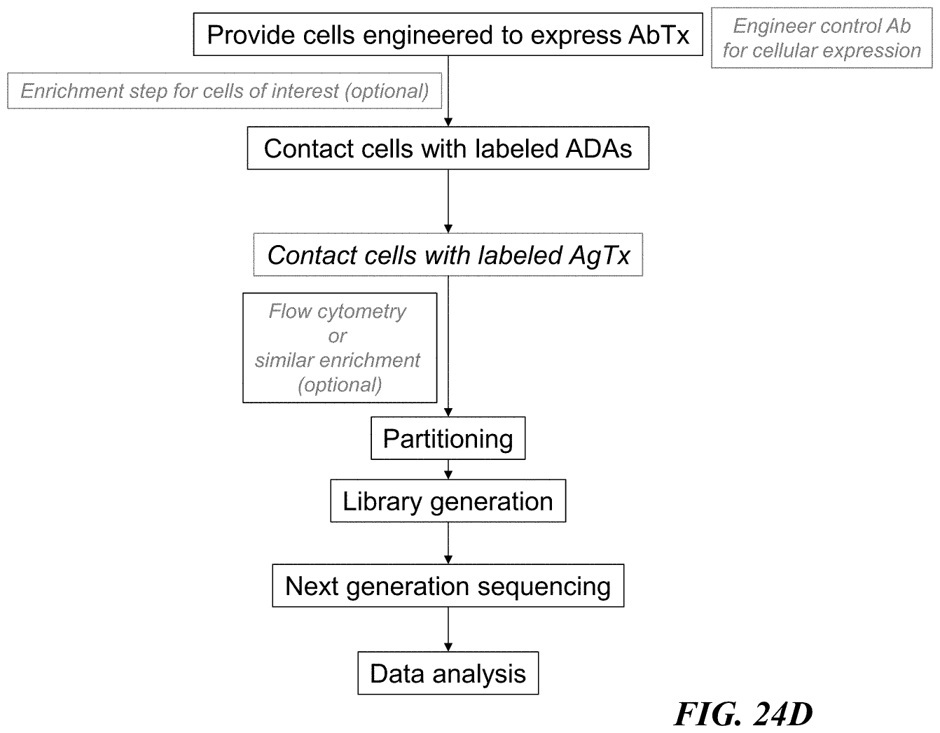

The methods provided herein can be implemented to identify ADAs to an AbTx via barcoding. An exemplary workflow of identifying ADAs is described in FIG. 24A. A sample can be obtained from a patient treated with a known AbTx. For example, peripheral blood mononuclear cells (PBMCs) can be isolated from the patient treated with the AbTx. Alternatively, a cellular yeast or mammalian cell display library expressing a library of antibodies can be used in place of PBMCs. This display library can comprise a library of antibodies derived from the patient treated with the AbTx.

In an embodiment, when the sample is the isolated PBMCs, the sample can be enriched for B cells or T cells with a commercially available kit.

The known AbTx can be conjugated to a reporter oligonucleotide according to the methods described herein. In some embodiments, the AbTx can also be labeled with a reporter fluorophore or any other detectable labels that can enable identification of the AbTx by, for example, flow cytometry or any other known similar methods. In some embodiments, antigen-specific enrichment of B cells can be performed using flow cytometry. In some embodiments, the AbTx can be pre-incubated with its labeled antigenic target (AgTx), enabling identification of ADAs binding to AbTx-AgTx complex rather than the AbTx alone.

The cells can be incubated with the reporter oligonucleotide conjugated AbTx. In some embodiments, the cells can be contacted with a reporter oligonucleotide conjugated AbTx panel comprising unmodified and modified (or mutated) AbTx conjugated with reporter oligonucleotides. The cells bound to the reporter oligonucleotide conjugated AbTx are partitioned and subjected to, for example, 10×5′V2 Single Cell Immune Profiling kit per manufacturer's instructions. Additional information in this regard can be found at “support.10×genomics.com/permalink/getting-started-immune-profiling-feature-barcoding.” This can generate a reporter barcode library, a V(D)J library, and optional a gene expression library. The reporter barcode library can include counts of AbTx, AgTx, and/or ADA. The V(D)J library can include native sequences of ADAs or expressed antibody sequences in engineered cells.

The generated libraries can be sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®).

A count matrix of cells expressing ADAs and reporter barcodes associated with the AbTx or AbTx-AgTx complex can be generated from the sequence data. In some embodiments, the count matrix is a count matrix described in FIG. 16, wherein each row corresponds to a cell expressing an antigen binding molecule (i.e., an ADA), and each column corresponds to an antigen (i.e., the known AbTx (unmodified), one or more modified (mutated) AbTx with at least one point mutation, or the AbTx-AgTx complex). Each element of the count matrix described in FIG. 16 corresponds to a count of quantity of times the antigen bound to a cell expressing a particular antigen binding molecule (i.e., a particular ADA). BCR or TCR sequences from cells having a threshold number of AbTx reporter oligonucleotide UMI counts can be used to identify the particular ADA bound to the unmodified AbTx, modified AbTx, or the AbTx-AgTx complex.

### Identification of a Patient-Specific Antibody Therapeutic

The methods provided herein can be implemented to identify a patient specific engineered AbTx that does not elicit an immune response in the patient. An exemplary workflow of identifying the patient specific AbTx is described in FIG. 24B.

The ADAs identified according to the methods disclosed herein, e.g., the methods disclosed in Example 10, can be conjugated to reporter oligonucleotides as described herein. In some embodiments, the ADAs can also be labeled with a reporter fluorophore or any other detectable labels that can enable identification of the ADA by, for example flow cytometry or any other known similar methods.

Cells can be engineered to express the AbTx and/or the AbTx comprising one or more point mutations. In some embodiments, the AbTx and/or the modified (mutated) AbTx can be tethered to the cell surface when expressed from constructs comprising the 5th exon, but not the 6th exon, of an IgG1 constant region. In some embodiments, the AbTx and/or the modified (mutated) AbTx can be secreted when expressed from constructs comprising the 6th exon, but not the 5th exon, of an IgG1 constant region. In some embodiments, the engineered cells expressing the AbTx or the modified (mutated) AbTx can be pre-incubated with its labeled antigenic target (AgTx), thereby forming a AbTx-AgTx complex. In some embodiments, the engineered cells can be enriched in order to identify a modified (mutated) AbTx with a low ADA activity or a high ADA activity.

The engineered cells can be incubated with the reporter oligonucleotide conjugated ADAs. In some embodiments, the engineered cells can be contacted with a panel of reporter oligonucleotide conjugated ADAs and a control antibody.

The engineered cells bound to the reporter oligonucleotide conjugated ADAs can be partitioned and subjected to, for example, 10×5′V2 Single Cell Immune Profiling kit per manufacturer's instructions. Additional information in this regard can be found at “support.10×genomics.com/permalink/getting-started-immune-profiling-feature-barcoding.” This can generate a reporter barcode library, a V(D)J library, and optional a gene expression library. The AbTx or modified (mutated) AbTx can be detected by V(D)J chemistry or by targeted GEX chemistry. Reporter barcoded ADAs can be detected by the reporter barcode chemistry. Reporter barcoded AbTx-AgTx can be detected by the reporter barcode chemistry.

The libraries can be enriched for targets of interest, which can include any combination of ADA antibody sequences identified using methods disclosed herein, and known AbTx or AbTx mutant sequences. An exemplary target enrichment method can comprise providing a plurality of barcoded nucleic acid molecules (e.g., library members) and hybridizing barcoded nucleic acid molecules comprising targeted regions of interest to oligonucleotide probes (“baits”) which can be complementary to the targeted regions of interest (or to regions near or adjacent to the targeted regions of interest). Baits can be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

The generated libraries can be sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®). The binding affinity of ADAs to the AbTx, the modified (mutated) AbTx, or the AbTx-AgTx complex can be determined based on quantity/numbers of unique molecular identifiers (UMIs) associated with each of the antigen binding molecules bound to the target antigen.

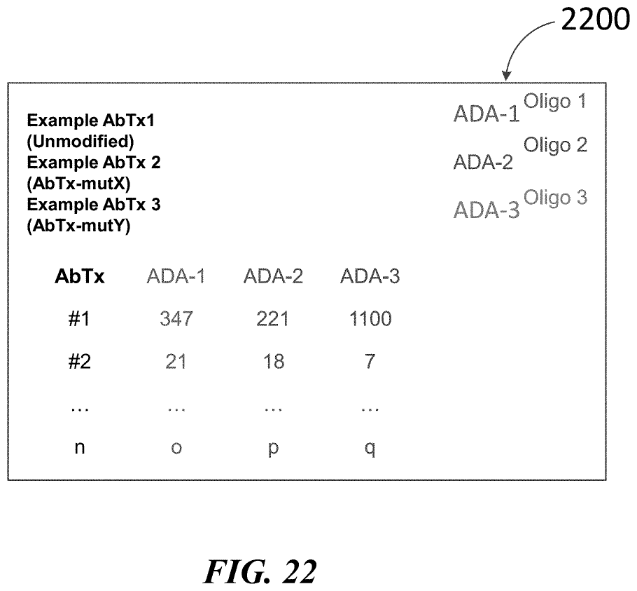

A count matrix of cells expressing the AbTx or the modified (mutated) AbTx and reporter barcodes associated with the ADAs can be generated. In some embodiments, the count matrix is a count matrix described in FIG. 22, wherein each row corresponds to a cell expressing an antigen binding molecule (i.e., the known AbTx (unmodified), one or more modified (mutated) AbTx with at least one point mutation, or the AbTx-AgTx complex), and each column corresponds to an antigen (i.e., an ADA). Each element of the count matrix described in FIG. 22 corresponds to a count of quantity of times the antigen bound to a cell expressing a particular antigen binding molecule (i.e., a particular AbTx). This data can be used to identify the epitope (the antigen binding site) of the unmodified AbTx, the modified (mutated) AbTx, or the AbTx-AgTx bound by the ADA. This data can be used to identify a patient-specific AbTx, engineered to not elicit an immune response in the patient when treated with the identified patient-specific AbTx.

### Characterizing Binding of Anti-Drug Antibody to Antibody Therapeutic

The methods provided herein can be implemented to identify a patient specific engineered AbTx that does not elicit an immune response in patient. In some cases, the methods identify a patient-specific engineered AbTx that retains its binding profile to its AgTx and does not elicit an immune response in the patient. Exemplary workflows of identifying the patient specific AbTx are described in FIGS. 24C-24D.

Referring to FIG. 24C, the AbTx can be conjugated to reporter oligonucleotides as described herein. Optionally, the AbTx is modified (mutated) to comprise at least one mutation, wherein each modified AbTx is conjugated with a unique reporter oligonucleotide. For example, a panel of AbTx's, e.g., including an unmodified AbTx and one or more modified AbTx's, can be conjugated with reporter oligonucleotides that identify the AbTx. The AbTx and/or modified AbTx can also be labeled with a reporter fluorophore or any other detectable labels that can enable identification of the AbTx by, for example flow cytometry or any other known similar methods. In some embodiments, the AbTx and/or modified AbTx can be also labeled with an enrichment molecule, which can, for example, without limitation, activate FRET-based fluorescence upon binding its target ligand. In some embodiments, the target ligand can also be labeled with a reporter oligonucleotide or a fluorescent molecule that can be used in downstream enrichment steps.

Continuing with FIG. 24C, cells can be engineered to express the ADAs identified in Example 10. Optionally, cells engineered to express a control antibody can be provided. The control antibody can be a non-ADA that can bind an irrelevant target that is not the AbTx (e.g., a cell surface protein, a murine version of a human protein). The control antibodies can be a commercially available clone against, for example, without limitation, CD3 or CD8. In some embodiments, the ADAs can be tethered to the cell surface when expressed from constructs comprising the 5th exon, but not the 6th exon, of an IgG1 constant region. In some embodiments, the ADAs can be secreted when expressed from constructs comprising the 6th exon, but not the 5th exon, of an IgG1 constant region.

The engineered cells can be incubated with the AbTx and/or the modified (mutated) AbTx conjugated with reporter oligonucleotides. In some embodiments, the engineered cells can be contacted with a panel of the AbTx and/or the modified (mutated) AbTx conjugated with reporter oligonucleotides. In some embodiments, the engineered cells expressing the ADAs can be pre-incubated with its labeled antigenic target, which can optionally comprise a reporter oligonucleotide that can identify the antigenic target. In some embodiments, the engineered cells can be enriched in order to identify a modified (mutated) AbTx that poorly or strongly binds ADAs. This enrichment process can optionally be repeated in a panning-like process.

The engineered cells bound to the AbTx and/or modified (mutated) AbTx conjugated with reporter oligonucleotides are partitioned and subjected to, for example, 10×5′V2 Single Cell Immune Profiling kit per manufacturer's instructions. Additional information in this regard can be found at “support.10×genomics.com/permalink/getting-started-immune-profiling-feature-barcoding.” This can generate a reporter barcode library, a V(D)J library, and optional a gene expression library. The reporter barcode library can be used to detect the presence of the AbTx, the modified (mutated) AbTx, and optionally the AgTx ligand. The V(D)J library and the gene expression library can be used to detect the presence of ADAs and the control antibodies expressed by the cells. The libraries can be enriched for targets of interest, which can include any combination of ADA antibody sequences identified using methods disclosed herein, and known AbTx or AbTx mutant sequences. Exemplary methods for enrichment of libraries for targets of interest are disclosed herein.

The generated libraries can be sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®). The binding affinity of ADAs to the AbTx, the modified (mutated) AbTx, or the AbTx-AgTx complex can be determined based on quantity/numbers of unique molecular identifiers (UMIs) associated with each of the antigen binding molecules bound to the target antigen.

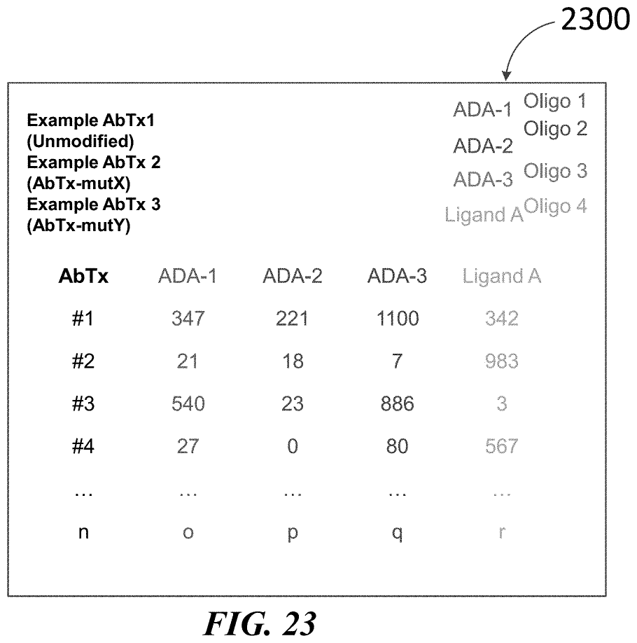

A count matrix of cells expressing the ADAs and the AbTx or the modified (mutated) AbTx conjugated with and reporter barcodes can be generated. In some embodiments, the count matrix is a count matrix described in FIG. 23, wherein each row corresponds to a cell expressing an antigen binding molecule (i.e., the known AbTx (unmodified), one or more modified (mutated) AbTx with at least one point mutation, or the AbTx-AgTx complex), and each column corresponds to an antigen (i.e., an ADA). Each element of the count matrix described in FIG. 23 corresponds to a count of quantity of times the antigen bound to a cell expressing a particular antigen binding molecule (i.e., a particular AbTx). This data can be used to identify (1) cases where each AbTx or each modified (mutated) AbTx is bound by one or more ADAs; (2) whether each AbTx or each modified (mutated) AbTx binds the AgTx ligand; (3) whether an ADA binding of each AbTx or each modified (mutated) AbTx can abrogate AgTx ligand binding; and (4) whether a particular AbTx-ADA binding is dependent on a AgTx binding to AbTx.

The ADAs and AbTx can be reversed in this workflow, where a library of ADAs can bind the cells expressing the AbTx and/or the modified (mutated) AbTx, the process of which is described in FIG. 24D.

### Determining an Epitope

In some embodiments, methods provided herein can include identifying a site on the antigen binding to the antigen binding molecule. Identification of a site can include identification of at least one amino acid(s) that contribute of binding of the antigen to the antigen binding molecule.

In some embodiments, methods provided herein can include identifying an epitope on an antigen that has affinity to the antigen binding molecule. Such an epitope can partially or fully confer affinity of the antigen binding molecule to the antigen.

An epitope can be a portion of an antigen or other macromolecule capable of forming a binding interaction with the variable region binding pocket of an antigen binding molecule such as an antibody or antigen binding fragment thereof. Such binding interactions can be manifested as an intermolecular contact with one or more amino acid residues of one or more CDRs. Antigen binding can involve, for example, a CDR3, a CDR3 pair or, in some cases, interactions of up to all six CDRs of the V_{H} and V_{L} chains. In some embodiments, antigen binding can involve framework regions (FWRs) that scaffold the CDRs. An epitope can be a linear peptide sequence (i.e., “continuous”) or can be composed of noncontiguous amino acid sequences (i.e., “conformational” or “discontinuous”). An antigen binding molecule such as an antibody or antigen binding fragment thereof can recognize one or more amino acid sequences; therefore, an epitope can define more than one distinct amino acid sequence. Epitopes recognized by antigen binding molecules such as antibodies or antigen binding fragments thereof can be determined by peptide mapping or sequence analysis techniques. In some embodiments, binding interactions can be manifested as intermolecular contacts between an epitope on an antigen and one or more amino acid residues of a CDR. Epitopes recognized by antigen binding molecules such as antibodies or antigen binding fragments thereof can be determined, for example, by peptide mapping or sequence analysis techniques. Binding interactions can manifest as intermolecular contacts between an epitope on an antigen and one or more amino acid residues of a complementarity determining region (CDR).

An epitope can be determined, for example, using one or more epitope mapping techniques. Epitope mapping can include experimentally identifying the epitope on an antigen. Epitope mapping can be performed by any acceptable method, for example, but not limited to, X-ray co-crystallography, cryogenic electron microscopy, array-based oligo-peptide scanning, site-directed mutagenesis mapping, high-throughput shotgun mutagenesis epitope mapping, hydrogen-deuterium exchange, cross-linking coupled mass spectrometry, yeast display, phage display, proteolysis, SPR chip, next generation sequencing, or a combination thereof.

### Epitope Modification

In some embodiments, methods provided herein can include modifying an epitope of the antigen. In some embodiments, an epitope can be modified to reduce the affinity of the antigen binding molecule for the antigen. In some such cases, an epitope can be modified to partially or fully prevent binding of the antigen binding molecule to the antigen.

In some embodiments, modification of an epitope can be based on the identity of the antigen binding molecule or the epitope. In some cases, an epitope determined, for example by epitope mapping as provided herein, can be modified. After modification of the epitope, the modified antigen can be contacted with the antigen binding molecule, and affinity between the modified antigen and antigen binding molecule can be determined. In some embodiments, the affinity between the modified antigen and antigen binding molecule can be significantly reduced or physiologically negligible.

Epitope modification can include one or more of: a modification (e.g., insertion, deletion, or mutation) of the amino acid sequence of the epitope, a post-translational modification to the epitope (e.g., glycosylation, ubiquitination, phosphorylation, myristoylation, palmitoylation, isoprenylation, farnesylation, geranylgeranylation, glipyation, lipoylation, attachment of a flavin moiety, attachment of heme C, phosphopantetheinylation, retinylidene Schiff base formation, diphthamide formation, ethanolamide phosphoglycerol attachment, hypusine formation, beta-Lysine addition, acylation, alkylation, amidation, amide bond formation, arginylation, polyglutamylation, polyglycylation, butyrylation, gamma-carboxylation, malonylation, hydroxylation, iodination, nucleotide addition, phosphate ester or phosphoramidate formation, adenylation, uridylylation, propionylation, pyroglutamate formation, S-glutathionylation, S-nitrosylation, S-sulfenylation, S-sulfinylation, succinylation, sulfation, glycation, carbamylation, carbonylation, spontaneous isopeptide bond formation, biotinylation, carbamylation, oxidation, pegylation, ISGylation, SUMOylation, neddylation, pupylation, citrullination, deamidation, eliminylation, formation of a disulfide bridge, proteolytic cleavage, isoaspartate formation, racemization, protein splicing, or a combination thereof), or binding of a molecule to the epitope to block the antigen binding molecule from binding the epitope.

For example, an antigen binding molecule such as an antibody from a subject can be identified as having affinity to an antibody (e.g., an antibody therapeutic) or antibody-based drug to be administered to the subject. In some embodiments, administering that antibody or antibody-based drug to the subject can result in binding of the antibody (e.g., an antibody therapeutic) or antibody-based drug to the binding molecule in the subject, thereby reducing the therapeutic effect of the antibody (e.g., an antibody therapeutic) or antibody-based drug, undesirable side effects, or other undesirable effects. In some embodiments, an epitope of the antibody (e.g., an antibody therapeutic) or antibody-based drug can be modified such that the affinity of one or more antigen binding molecules of the subject for the antibody (e.g., an antibody therapeutic) or antibody-based drug is reduced. In some such cases, reduction in therapeutic effect of the antibody (e.g., an antibody therapeutic) or antibody-based drug, undesirable side effects, or other undesirable effects can be ameliorated. In some embodiments, the epitope can be modified without significantly reducing the efficacy of the antibody (e.g., an antibody therapeutic) or antibody-based drug. In some embodiments, the modification to the epitope of the antibody (e.g., antibody therapeutic) or antibody-based drug comprises a modification to one or more framework (FWR) or CDR sequences of the antibody (e.g., antibody therapeutic) or antibody-based drug, such as CDRH3 or CDRL3. In some embodiments, these modifications to the heavy chain or light chain FWR and CDR sequences of the antibody (e.g., antibody therapeutic) or antibody-based drug does not affect its binding to the antigenic target (AgTx) of the antibody (e.g., antibody therapeutic) or antibody-based drug.

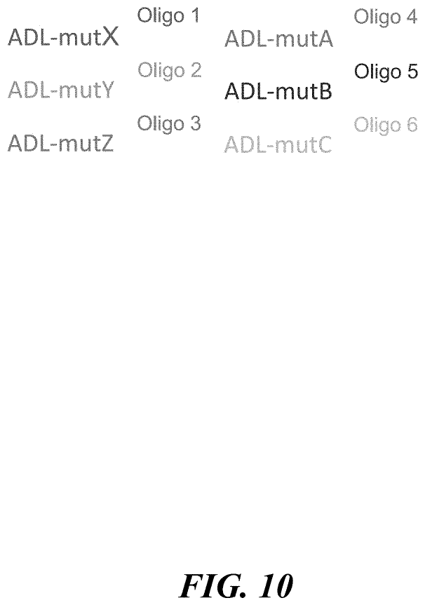

In some embodiments, one or more antigens having one or more modified epitopes (e.g., antigen variants) can be conjugated to reporter oligonucleotides and used as antigens in methods provided herein. In some embodiments, antigens having one or more modified epitopes can have reduced affinity for the antigen-binding molecule. In some embodiments, antigens having one or more modified epitopes can have no affinity for the antigen-binding molecule. Examples of such antigen variants conjugated to oligonucleotides are provided in FIG. 10.

### Antigens and Antigen Binding Molecules

### Antigens

An antigen can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof.

An antigen can be a molecule that can have affinity to an antigen binding molecule. For example, an antigen can have affinity to an antibody or antigen binding fragment thereof. In some, when contacted with an antigen binding molecule, the antigen can bind to the antigen binding molecule. In some embodiments, an antigen can be a biomolecule, such as a biologic therapeutic molecule. Examples of biologic therapeutic molecules can be, for example, a drug-reactive antibody or anti-drug antibody that is produced from a living organism or that contains one or more components of a living organism. A biologic therapeutic molecule can be derived from a human, animal, or microorganism using biotechnology techniques. Examples of biologic therapeutic molecules can include, for example, an immunological molecule (e.g. an antibody (such as a monoclonal antibodies), a fusion protein, a protein product of a gene therapy, a peptide, or other biologic molecule.

In some embodiments, an antigen can be an antibody or antigen binding fragment thereof. In some embodiments, an antigen can be an antibody-drug conjugate. In some embodiments, an antigen can be a therapeutic antibody or antigen binding fragment thereof (e.g., a monoclonal antibody). In some embodiments, an antigen can be a chimeric antigen receptor T cell therapy. In some embodiments, the chimeric antigen receptor T cell therapy is selected from the group consisting of axicabtagene ciloleucel (YESCARTA®), tisagenlecleucel (KYMRIAH®), brexucabtagene autoleucel (TECARTUS), idecabtagene vicleucel (ABECMA®), and lisocabtagene maraleucel. (BREYANZI®).

### Antigen Binding Molecules

An antigen binding molecule can be a molecule capable of binding an antigen. In some embodiments, an antigen binding molecule can be an antibody or antigen binding fragment thereof. Examples of antibodies are provided herein.

In some embodiments, an antigen binding molecule can be an antibody (such as an ADA) or antigen binding fragment thereof produced by a subject. In some embodiments, the antibody or antigen binding fragment thereof can have affinity to an antigen provided herein. In some embodiments, the antibody or antigen binding fragment thereof can have affinity to an antibody or antibody-based drug, for example an antibody or antibody-based drug that can be administered to a subject. In some embodiments, the antigen binding molecule can have affinity to an antigen that is a biologic or a small molecule. For example, in some embodiments, the antigen binding molecule can have affinity to a component of a vaccine composition.

### Antibodies

In methods provided herein, an antigen or an antigen binding molecule can be an antibody or antigen-binding fragment thereof. As used herein, the term “antibody” can refer to an immunoglobulin (Ig), polypeptide, or a protein having a binding domain which is, or is homologous to, an antigen-binding domain. The term can further include “antigen-binding fragments” and other interchangeable terms for similar binding fragments as described herein.

In some embodiments, an antigen or an antigen binding molecule can be a therapeutic antibody or antigen binding fragment thereof (e.g., a monoclonal antibody). A therapeutic antibody or antigen binding fragment thereof can be a drug candidate or an FDA approved drug or therapeutic, such as a monoclonal antibody that is approved by the FDA for therapeutic use. Non-limiting examples of FDA approved monoclonal antibodies are provided in Table 1.

TABLE 1
FDA Approved Therapeutic Monoclonal Antibodies and other immunotherapies

| Antibody | Brand name | Type | Target |
|---|---|---|---|
| abciximab | ReoPro | chimeric Fab | GPIIb/IIIa |
| adalimumab | Humira | fully human | TNF |
| adalimumab-atto | Amjevita | fully | TNF |
| | | human, biosimilar | |
| ado-trastuzumab emtansine | Kadcyla | humanized, antibody- | HER2 |
| | | drug conjugate | |
| alemtuzumab | Campath, | humanized | CD52 |
| | Lemtrada | | |
| alirocumab | Praluent | fully human | PCSK9 |
| atezolizumab | Tecentriq | humanized | PD-L1 |
| atezolizumab | Tecentriq | humanized | PD-L1 |
| avelumab | Bavencio | fully human | PD-L1 |
| basiliximab | Simulect | chimeric | IL2RA |
| belimumab | Benlysta | fully human | BLyS |
| bevacizumab | Avastin | humanized | VEGF |
| bezlotoxumab | Zinplava | fully human | Clostridium |
| | | | difficile toxin B |
| blinatumomab | Blincyto | mouse, bispecific | CD19 |
| brentuximab vedotin | Adcetris | chimeric, antibody- | CD30 |
| | | drug conjugate | |
| brodalumab | Siliq | chimeric | IL17RA |
| canakinumab | Ilaris | fully human | IL1B |
| capromab pendetide | ProstaScint | murine, radiolabeled | PSMA |
| certolizumab pegol | Cimzia | humanized | TNF |
| cetuximab | Erbitux | chimeric | EGFR |
| daclizumab | Zenapax | humanized | IL2RA |
| daclizumab | Zinbryta | humanized | IL2R |
| daratumumab | Darzalex | fully human | CD38 |
| denosumab | Prolia, Xgeva | fully human | RANKL |
| dinutuximab | Unituxin | chimeric | GD2 |
| dupilumab | Dupixent | fully human | IL4RA |
| durvalumab | Imfinzi | fully human | PD-L1 |
| eculizumab | Soliris | humanized | Complement |
| | | | component 5 |
| elotuzumab | Empliciti | humanized | SLAMF7 |
| evolocumab | Repatha | fully human | PCSK9 |
| golimumab | Simponi | fully human | TNF |
| golimumab | Simponi Aria | fully human | TNF |
| ibritumomab tiuxetan | Zevalin | murine, | CD20 |
| | | radioimmunotherapy | |
| idarucizumab | Praxbind | humanized Fab | dabigatran |
| infliximab | Remicade | chimeric | TNF alpha |
| infliximab-abda | Renflexis | chimeric, biosimilar | TNF |
| infliximab-dyyb | Inflectra | chimeric, biosimilar | TNF |
| ipilimumab | Yervoy | fully human | CTLA-4 |
| ixekizumab | Taltz | humanized | IL17A |
| mepolizumab | Nucala | humanized | IL5 |
| natalizumab | Tysabri | humanized | alpha-4 integrin |
| necitumumab | Portrazza | fully human | EGFR |
| nivolumab | Opdivo | fully human | PD-1 |
| nivolumab | Opdivo | fully human | PD-1 |
| obiltoxaximab | Anthem | chimeric | Protective |
| | | | antigen of |
| | | | the Anthrax |
| | | | toxin |
| obinutuzumab | Gazyva | humanized | CD20 |
| ocrelizumab | Ocrevus | humanized | CD20 |
| ofatumumab | Arzerra | fully human | CD20 |
| olaratumab | Lartruvo | fully human | PDGFRA |
| omalizumab | Xolair | humanized | IgE |
| palivizumab | Synagis | humanized | F protein |
| | | | of RSV |
| panitumumab | Vectibix | fully human | EGFR |
| pembrolizumab | Keytruda | humanized | PD-1 |
| pertuzumab | Perjeta | humanized | HER2 |
| ramucirumab | Cyramza | fully human | VEGFR2 |
| ranibizumab | Lucentis | humanized | VEGFR1, |
| | | | VEGFR2 |
| raxibacumab | Raxibacumab | fully human | Protective |
| | | | antigen |
| | | | of Bacillus |
| | | | anthracis |
| reslizumab | Cinqair | humanized | IL5 |
| rituximab | Rituxan | chimeric | CD20 |
| secukinumab | Cosentyx | fully human | IL17A |
| siltuximab | Sylvant | chimeric | IL6 |
| tocilizumab | Actemra | humanized | IL6R |
| tocilizumab | Actemra | humanized | IL6R |
| trastuzumab | Herceptin | humanized | HER2 |
| ustekinumab | Stelara | fully human | IL12 |
| ustekinumab | Stelara | fully human | IL12, IL23 |
| vedolizumab | Entyvio | humanized | integrin receptor |
| sarilumab | Kevzara | fully human | IL6R |
| rituximab and hyaluronidase | Rituxan | chimeric, co- | CD20 |
| | Hycela | formulated | |
| guselkumab | Tremfya | fully human | IL23 |
| inotuzumab ozogamicin | Besponsa | humanized, antibody- | CD22 |
| | | drug conjugate | |
| adalimumab-adbm | Cyltezo | fully | TNF |
| | | human, biosimilar | |
| gemtuzumab ozogamicin | Mylotarg | humanized, antibody- | CD33 |
| | | drug conjugate | |
| bevacizumab-awwb | Mvasi | humanized, biosimilar | VEGF |
| benralizumab | Fasenra | humanized | interleukin-5 |
| | | | receptor alpha |
| | | | subunit |
| emicizumab-kxwh | Hemlibra | humanized, bispecific | Factor |
| | | | IXa, Factor X |
| trastuzumab-dkst | Ogivri | humanized, biosimilar | HER2 |
| infliximab-qbtx | Ixifi | chimeric, biosimilar | TNF |
| ibalizumab-uiyk | Trogarzo | humanized | CD4 |
| tildrakizumab-asmn | Ilumya | humanized | IL23 |
| burosumab-twza | Crysvita | fully human | FGF23 |
| erenumab-aooe | Aimovig | fully human | CGRP receptor |

In some embodiments, an antigen or an antigen binding molecule can be similar to an FDA approved therapeutic monoclonal antibody. In some such cases, an antigen or an antigen binding molecule can have at least 75% identity, at least 80% identity, at least 85% identity, at least 90% identity, or at least 95% identity to an FDA approved therapeutic monoclonal antibody or a range between any two foregoing values. In some embodiments, an antigen or an antigen binding molecule can have a heavy chain variable region that is at least 75% identity, at least 80% identity, at least 85% identity, at least 90% identity, or at least 95% identity to the heavy chain variable region of an FDA approved therapeutic monoclonal antibody or a range between any two foregoing values. In some embodiments, an antigen or an antigen binding molecule can have a heavy chain constant region that is at least 75% identity, at least 80% identity, at least 85% identity, at least 90% identity, or at least 95% identity to the heavy chain constant region of an FDA approved therapeutic monoclonal antibody or a range between any two foregoing values. In some embodiments, an antigen or an antigen binding molecule can have a light chain variable region that is at least 75% identity, at least 80% identity, at least 85% identity, at least 90% identity, or at least 95% identity to a light chain variable region of an FDA approved therapeutic monoclonal antibody or a range between any two foregoing values. In some embodiments, an antigen or an antigen binding molecule can have a light chain constant region that is at least 75% identity, at least 80% identity, at least 85% identity, at least 90% identity, or at least 95% identity to the light chain constant region of an FDA approved therapeutic monoclonal antibody or a range between any two foregoing values. In some embodiments, an antigen or an antigen binding molecule can be an antibody or antigen binding fragment thereof that has a target that is the same as the target of an FDA approved therapeutic monoclonal antibody. In some embodiments, the antibody can be a secreted antibody. In some embodiments, the antibody can be a surface-bound antibody.

Native antibodies and native immunoglobulins (Igs) can be heterotetrameric glycoproteins of about 150,000 Daltons, composed of two identical light chains and two identical heavy chains. Antibodies can further refer to camelid antibodies, which can be not tetrameric. Each light chain can be typically linked to a heavy chain by one covalent disulfide bond, while the number of disulfide linkages can vary among the heavy chains of different immunoglobulin isotypes. Each heavy and light chain can have regularly spaced intrachain disulfide bridges. Each heavy chain can have at one end a variable domain (“V_{H}”) followed by a number of constant domains (“C_{H}”). Each light chain can have a variable domain at one end (“V_{L}”) and a constant domain (“C_{L}”) at its other end; the constant domain of the light chain can be aligned with the first constant domain of the heavy chain, and the light-chain variable domain can be aligned with the variable domain of the heavy chain. Particular amino acid residues can form an interface between the light- and heavy-chain variable domains.

In some instances, an antibody or an antigen-binding fragment thereof includes an isolated antibody or antigen-binding fragment thereof, a purified antibody or antigen-binding fragment thereof, a recombinant antibody or antigen-binding fragment thereof, a modified antibody or antigen-binding fragment thereof, or a synthetic antibody or antigen-binding fragment thereof.

Antibodies and antigen-binding fragments herein can be partly or wholly synthetically produced. An antibody or antigen-binding fragment can be a polypeptide or protein having a binding domain which can be, or can be homologous to, an antigen binding domain. In some instances, an antibody or an antigen-binding fragment thereof can be produced in an appropriate in vivo animal model and then isolated and/or purified.

Depending on the amino acid sequence of the constant domain of its heavy chains, immunoglobulins (Igs) can be assigned to different classes. Major classes of immunoglobulins can include: IgA, IgD, IgE, IgG, and IgM, and several of these may be further divided into subclasses (isotypes), e.g., IgG1, IgG2, IgG3, IgG4, IgA1 and IgA2. An Ig or portion thereof can, in some cases, be a human Ig. In some instances, a C_{H}3 domain can be from an immunoglobulin. In some cases, a chain or a part of an antibody or antigen binding fragment thereof, a modified antibody or antigen-binding fragment thereof, or a binding agent can be from an Ig. In such cases, an Ig can be IgG, an IgA, an IgD, an IgE, or an IgM. In cases where the Ig is an IgG, it can be a subtype of IgG, wherein subtypes of IgG can include IgG1, an IgG2a, an IgG2b, an IgG3, and an IgG4. In some cases, a C_{H}3 domain can be from an immunoglobulin selected from the group consisting of an IgG, an IgA, an IgD, an IgE, and an IgM.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody can refer to the variable region of the antibody light chain or the variable region of the antibody heavy chain, either alone or in combination. The variable regions of the heavy and light chain can consist of four framework regions (FR) connected by three complementarity determining regions (CDRs) also known as hypervariable regions. The CDRs in each chain can be held together in close proximity by the FRs and, with the CDRs from the other chain, contribute to the formation of the antigen-binding site of antibodies. CDRs can be determined by methods such as: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” can refer to the variable domains of antibodies that are used in the binding and specificity of each particular antibody for its particular antigen. In some cases, the variability is not evenly distributed throughout the variable domains of antibodies. In some cases, it is concentrated in three segments called hypervariable regions (also known as CDRs) in both the light chain and the heavy chain variable domains. More highly conserved portions of variable domains can be called the “framework regions” or “FWRs” or “FRs.” The variable domains of unmodified heavy and light chains can contain four FRs (FR1, FR2, FR3, and FR4), largely adopting a β-sheet configuration interspersed with three CDRs which can form loops connecting and, in some cases, part of the β-sheet structure. The CDRs in each chain can be held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669).

“Antibodies” useful in the present disclosure can encompass monoclonal antibodies, polyclonal antibodies, chimeric antibodies, bispecific antibodies, multispecific antibodies, heteroconjugate antibodies, humanized antibodies, human antibodies, deimmunized antibodies, mutants thereof, fusions thereof, immunoconjugates thereof, antigen-binding fragments thereof, and/or any other modified configuration of the immunoglobulin molecule that includes an antigen recognition site of the required specificity, including glycosylation variants of antibodies, amino acid sequence variants of antibodies, and covalently modified antibodies. In some embodiments, an antibody can be a murine antibody.

An antibody can be a human antibody. As used herein, a “human antibody” can be an antibody having an amino acid sequence corresponding to that of an antibody produced by a human and/or that has been made using any suitable technique for making human antibodies. Human antibodies can include antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies (Vaughan et al., 1996, Nature Biotechnology, 14:309-314; Sheets et al., 1998, PNAS USA, 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

Any of the antibodies herein can be bispecific. Bispecific antibodies can be antibodies that have binding specificities for at least two different antigens and can be prepared using the antibodies disclosed herein. Exemplary methods for making bispecific antibodies are described (see, e.g., Suresh et al., 1986, Methods in Enzymology 121:210). The recombinant production of bispecific antibodies can be based on the coexpression of two immunoglobulin heavy chain-light chain pairs, with the two heavy chains having different specificities (Millstein and Cuello, 1983, Nature, 305, 537-539). Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, can facilitate separation of the desired bispecific compound from unwanted immunoglobulin chain combinations. This approach is described, for example, in PCT Publication No. WO 94/04690.

Functional fragments of any of the antibodies herein are also contemplated. The terms “antigen-binding portion of an antibody,” “antigen-binding fragment,” “antigen-binding domain,” “antibody fragment,” or a “functional fragment of an antibody” can refer to one or more fragments of an antibody that retain the ability to specifically bind to an antigen. Representative antigen-binding fragments include a Fab, a Fab′, a F(ab′)₂, a Fv, a scFv, a dsFv, a variable heavy domain, a variable light domain, a variable NAR domain, bi-specific scFv, a bi-specific Fab2, a tri-specific Fab3, an AVIMER®, a minibody, a diabody, a maxibody, a camelid, a VHH, a minibody, an intrabody, fusion proteins comprising an antibody portion (e.g., a domain antibody), and a single chain binding polypeptide.

“F(ab′)₂” and “Fab′” moieties can be produced by treating an Ig with a protease such as pepsin and papain, and include antibody fragments generated by digesting immunoglobulin near the disulfide bonds existing between the hinge regions in each of the two heavy chains. For example, papain can cleave IgG upstream of the disulfide bonds existing between the hinge regions in each of the two heavy chains to generate two homologous antibody fragments in which an light chain composed of V_{L} and C_{L} (light chain constant region), and a heavy chain fragment composed of V_{H} and C_{Hγ1} (γ1) region in the constant region of the heavy chain) are connected at their C terminal regions through a disulfide bond. Each of these two homologous antibody fragments can be called Fab′. Pepsin can also cleave IgG downstream of the disulfide bonds existing between the hinge regions in each of the two heavy chains to generate an antibody fragment slightly larger than the fragment in which the two above-mentioned Fab′ are connected at the hinge region. This antibody fragment can be called F(ab′)2.

The Fab fragment can also contain the constant domain of the light chain and the first constant domain (C_{H}1) of the heavy chain. Fab′ fragments can differ from Fab fragments by the addition of a few residues at the carboxyl terminus of the heavy chain C_{H}1 domain including one or more cysteine(s) from the antibody hinge region. Fab′-SH can be a Fab′ in which the cysteine residue(s) of the constant domains bear a free thiol group. F(ab′)₂ antibody fragments can be produced, for example, as pairs of Fab′ fragments which have hinge cysteines between them. Other chemical couplings of antibody fragments can also be employed.

A “Fv” as used herein can refer to an antibody fragment which contains a complete antigen-recognition and antigen-binding site. This region can consist of a dimer of one heavy chain and one light chain variable domain in tight, non-covalent or covalent association (disulfide linked Fvs have been described, see, e.g., Reiter et al. (1996) Nature Biotechnology 14:1239-1245). In this configuration that the three CDRs of each variable domain can interact to define an antigen-binding site on the surface of the V_{H}-V_{L} dimer. Collectively, a combination of one or more of the CDRs can from each of the V_{H} and V_{L} chains confer antigen-binding specificity to the antibody. For example, the CDRH3 and CDRL3 can be sufficient to confer antigen-binding specificity to an antibody when transferred to V_{H} and V_{L} chains of a recipient antibody or antigen-binding fragment thereof and this combination of CDRs can be tested for binding, specificity, affinity, etc. using, for example, techniques described herein. In some cases, even a single variable domain (or half of an Fv comprising only three CDRs specific for an antigen) can have the ability to recognize and bind antigen, although likely at a lower specificity or affinity than when combined with a second variable domain. Furthermore, although the two domains of a Fv fragment (V_{L} and V_{H}) can be coded for by separate genes, they can be joined using recombinant methods, for example by a synthetic linker that enables them to be made as a single protein chain in which the V_{L} and V_{H} regions pair to form monovalent molecules (known as single chain Fv (scFv); Bird et al. (1988) Science 242:423-426; Huston et al. (1988) Proc. Natl. Acad. Sci. USA 85:5879-5883; and Osbourn et al. (1998) Nat. Biotechnol. 16:778). Such scFvs can be encompassed within the term “antigen-binding portion” of an antibody. Any V_{H} and V_{L} sequences of specific scFv can be linked to an Fc region cDNA or genomic sequences in order to generate expression vectors encoding complete Ig (e.g., IgG) molecules or other isotypes. V_{H} and V_{L} can also be used in the generation of Fab, Fv, or other fragments of Igs using either protein chemistry or recombinant DNA technology.

“Single-chain Fv” or “sFv” antibody fragments can include the V_{H} and V_{L} domains of an antibody, wherein these domains can be present in a single polypeptide chain. In some embodiments, the Fv polypeptide can further include a polypeptide linker between the V_{H} and V_{L} domains which enables the sFv to form the desired structure for antigen binding. For a review of sFvs, see, e.g., Pluckthun in The Pharmacology of Monoclonal Antibodies, Vol. 113, Rosenburg and Moore eds. Springer-Verlag, New York, pp. 269-315 (1994).

Also contemplated herein is the use of an AVIMER® as an antigen or antigen binding moiety. The term “AVIMER®” can refer to a class of therapeutic proteins of human origin, which can be unrelated to antibodies and antibody fragments, and can be composed of several modular and reusable binding domains, referred to as A-domains (also referred to as class A module, complement type repeat, or LDL-receptor class A domain). They can be developed from human extracellular receptor domains by in vitro exon shuffling and phage display (Silverman et al., 2005, Nat. Biotechnol. 23:1493-1494; Silverman et al., 2006, Nat. Biotechnol. 24:220). The resulting proteins can contain multiple independent binding domains that can exhibit improved affinity and/or specificity compared with single-epitope binding proteins. Each of the known 217 human A-domains can include ˜35 amino acids (˜4 kDa); and these domains can be separated by linkers that can average five amino acids in length. Native A-domains fold quickly and efficiently to a uniform, stable structure mediated primarily by calcium binding and disulfide formation. A conserved scaffold motif of only 12 amino acids can be required for this common structure. The end result can be a single protein chain containing multiple domains, each of which represents a separate function. Each domain of the proteins can bind independently, and the energetic contributions of each domain can be additive.

Antigen-binding polypeptides can also include heavy chain dimers such as, for example, antibodies from camelids and sharks. Camelid and shark antibodies can include a homodimeric pair of two chains of V-like and C-like domains (neither has a light chain). Since the V_{H} region of a heavy chain dimer IgG in a camelid does may not have to make hydrophobic interactions with a light chain, the region in the heavy chain that normally contacts a light chain can be changed to hydrophilic amino acid residues in a camelid. V_{H} domains of heavy-chain dimer IgGs can be called V_{HH} domains. Shark Ig-NARs can include a homodimer of one variable domain (termed a V-NAR domain) and five C-like constant domains (C-NAR domains). In camelids, the diversity of antibody repertoire can be determined by the CDRs 1, 2, and 3 in the V_{H} or V_{HH} regions. The CDR3 in the camel V_{HH} region can be characterized by its relatively long length, averaging 16 amino acids (Muyldermans et al., 1994, Protein Engineering 7(9): 1129). This can be in contrast to CDR3 regions of antibodies of many other species. For example, the CDR3 of mouse V_{H} can have an average of 9 amino acids. Libraries of camelid-derived antibody variable regions, which can maintain the in vivo diversity of the variable regions of a camelid, can be made by, for example, the methods disclosed in U.S. Patent Application Ser. No. 20050037421.

As used herein, a “maxibody” can refer to a bivalent scFv covalently attached to the Fc region of an immunoglobulin, see, e.g., Fredericks et al., Protein Engineering, Design & Selection, 17:95-106 (2004) and Powers et al., Journal of Immunological Methods, 251:123-135 (2001).

As used herein, a “dsFv” can be a Fv fragment obtained, for example, by introducing a Cys residue into a suitable site in each of a heavy chain variable region and a light chain variable region, and then stabilizing the heavy chain variable region and the light chain variable region by a disulfide bond. The site in each chain, into which the Cys residue can be introduced, can be determined based on a conformation predicted by molecular modeling. In the present disclosure, for example, a conformation can be predicted from the amino acid sequences of the heavy chain variable region and light chain variable region of the above-described antibody, and DNA encoding each of the heavy chain variable region and the light chain variable region, into which a mutation has been introduced based on such prediction, can be then constructed. The DNA construct can be incorporated then into a suitable vector and prepared from a transformant obtained by transformation with the aforementioned vector.

Single chain variable region fragments (“scFv”) of antibodies are described herein. Single chain variable region fragments may be made by linking light and/or heavy chain variable regions by using a short linking peptide. Bird et al. (1988) Science 242:423-426. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using any suitable protein purification techniques.

Diabodies can be single chain antibodies. Diabodies can be bivalent, bispecific antibodies in which V_{H} and V_{L} domains can be expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al., Proc. Natl. Acad. Sci. USA, 90:6444-6448 (1993); and Poljak, R. J., et al., Structure, 2:1121-1123 (1194)).

As used herein, a “minibody” can refer to a scFv fused to CH3 via a peptide linker (hingeless) or via an IgG hinge has been described in Olafsen, et al., Protein Eng Des Sel. April 2004; 17(4):315-23. As used herein, an “intrabody” can refer to a single chain antibody which can demonstrate intracellular expression and can manipulate intracellular protein function (Biocca, et al., EMBO J. 9:101-108, 1990; Colby et al., Proc Natl Acad. Sci. USA. 101:17616-21, 2004). Intrabodies, which can include cell signal sequences which can retain the antibody construct in intracellular regions, may be produced, for example, as described in Mhashilkar et al., (EMBO J., 14:1542-51, 1995) and Wheeler et al. (FASEB J. 17:1733-5. 2003). Transbodies can be cell-permeable antibodies in which a protein transduction domains (PTD) can be fused with single chain variable fragment (scFv) antibodies as in, for example, Heng et al. (Med Hypotheses. 64:1105-8, 2005).

### Systems and Methods for Partitioning

The systems and methods described herein, in an aspect, provide for the compartmentalization, depositing, or partitioning of one or more particles (e.g., biological particles or analyte careers, macromolecular constituents of biological particles, beads, reagents, etc.) into discrete compartments or partitions (referred to interchangeably herein as partitions), where each partition maintains separation of its own contents from the contents of other partitions. The terms “biological particles” and “analyte careers” are used interchangeably herein.

The term “partition,” as used herein, generally, refers to a space or volume that can be suitable to contain one or more species or conduct one or more reactions. A partition can be a physical compartment, such as a droplet or well. The partition can isolate space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

A partition can be a droplet in an emulsion. A partition can include one or more other partitions. A partition can include one or more particles. A partition can include one or more types of particles. For example, a partition of the present disclosure can include one or more biological particles or analyte careers and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition may not include a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, can be injected into the droplets previous to, subsequent to, or concurrently with droplet generation, such as via a cell bead, as described elsewhere herein.

The partitions can be flowable within fluid streams. The partitions can include, for example, micro-vesicles that have an outer barrier surrounding an inner fluid center or core. In some cases, the partitions can include a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In some instances, a droplet can be formed by creating an emulsion by mixing or agitating immiscible phases. Mixing or agitation can include various agitation techniques, such as vortexing, pipetting, tube flicking, or other agitation techniques. In some cases, mixing or agitation can be performed without using a microfluidic device. In some examples, a droplet can be formed by exposing a mixture to ultrasound or sonication. For example, to partition contents into droplets, a mixture including a first fluid, a second fluid, optionally a surfactant, and the contents can be subject to such agitation techniques to generate a plurality of droplets (first fluid-in-second fluid or second fluid-in-first fluid) including the contents, or subsets thereof. In an example, a mixture includes beads. Upon agitation, the beads in the mixture can limit droplet break-up into droplets smaller than the size of the beads, and a substantially monodisperse population of droplets comprising the beads can result.

In the case of droplets in an emulsion, allocating individual particles to discrete partitions can, in one non-limiting example, be accomplished by introducing a flowing stream of particles in an aqueous fluid into a flowing stream or reservoir of a non-aqueous fluid, such that droplets are generated at the junction of the two streams. By providing the aqueous cell-containing stream at a certain concentration level of cells, one can control the level of occupancy of the resulting partitions in terms of numbers of cells. Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles per partition, number of beads per partition, etc.). In some cases, where single cell partitions are desired, it may be desirable to control the relative flow rates of the fluids such that, on average, the partitions contain less than one cell per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions can contain at most one biological particle (e.g., bead, DNA, cell or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In many cases, the systems and methods provided herein are used to ensure that the substantial majority of occupied partitions include no more than one cell per occupied partition. In some cases, the partitioning process is controlled such that fewer than 25% of the occupied partitions contain more than one cell, and in many cases, fewer than 20% of the occupied partitions have more than one cell, while in some cases, fewer than 10% or even fewer than 5% of the occupied partitions include more than one cell per partition.

Additionally or alternatively, in many cases, it is desirable to avoid the creation of excessive numbers of empty partitions. While this can be accomplished by providing sufficient numbers of cells into the partitioning zone, the Poissonian distribution would expectedly increase the number of partitions that would include multiple cells. As such, in accordance with aspects described herein, the flow of one or more of the cells, or other fluids directed into the partitioning zone are controlled such that, in many cases, no more than 50% of the generated partitions are unoccupied, i.e., including less than one cell, no more than 25% of the generated partitions, no more than 10% of the generated partitions, may be unoccupied. Further, in some aspects, these flows are controlled so as to present non-Poissonian distribution of single occupied partitions while providing lower levels of unoccupied partitions. Restated, in some aspects, the above noted range of unoccupied partitions can be achieved while still providing any of the single occupancy rates described above. For example, in many cases, the use of the systems and methods described herein creates resulting partitions that have multiple occupancy rates of from less than 25%, less than 20%, less than 15%, less than 10%, and in many cases, less than 5%, while having unoccupied partitions of from less than 50%, less than 40%, less than 30%, less than 20%, less than 10%, and in some cases, less than 5%.

As will be appreciated, the above-described occupancy rates are also applicable to partitions that include both cells and beads carrying the nucleic acid barcode molecule. In particular, in some aspects, a substantial percentage of the overall occupied partitions will include both a bead and a cell. In particular, it may be desirable to provide that at least 50% of the partitions are occupied by at least one cell and at least one bead, or at least 75% of the partitions may be so occupied, or even at least 80%, or at least 90% of the partitions may be so occupied. Further, in those cases where it is desired to provide a single cell and a single bead within a partition, at least 50% of the partitions can be so occupied, at least 60%, at least 70%, at least 80%, or even at least 90% of the partitions can be so occupied. Methods and systems for generating desired occupancy rates of partitioning are described in U.S. Patent Publication No. 2015/0376609, which is entirely incorporated herein by reference for all purposes.

### Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure can include generating partitions and/or encapsulating particles, such as biological particles or analyte careers, in some cases, individual biological particles or analyte careers such as single cells. In some examples, reagents can be encapsulated and/or partitioned (e.g., co-partitioned with biological particles or analyte careers) in the partitions. Various mechanisms can be employed in the partitioning of individual particles. An example can include porous membranes through which aqueous mixtures of cells may be extruded into fluids (e.g., non-aqueous fluids).

The partitions can be flowable within fluid streams. The partitions can include, for example, micro-vesicles that have an outer barrier surrounding an inner fluid center or core. In some cases, the partitions can include a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions can be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters can be adjusted to control the occupancy of the resulting partitions (e.g., number of biological particles or analyte careers per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions can contain less than one biological particle per partition in order to ensure that those partitions that are occupied are primarily singly occupied. In some cases, partitions among a plurality of partitions can contain at most one biological particle (e.g., bead, DNA, cell or cellular material). In some embodiments, the various parameters (e.g., fluid properties, particle properties, microfluidic architectures, etc.) can be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

In some examples, a partition of the plurality of partitions can comprise a single biological particle (e.g., a single cell or a single nucleus of a cell). In some examples, a partition of the plurality of partitions can include multiple biological particles or analyte careers. Such partitions can be referred to as multiply occupied partitions, and can include, for example, two, three, four or more cells and/or beads (e.g., beads) including nucleic acid barcode molecules within a single partition. Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

### Microfluidic Channel Structures

Microfluidic channel networks (e.g., on a chip) can be utilized to generate partitions as described herein. Alternative mechanisms can also be employed in the partitioning of individual biological particles or analyte careers, including porous membranes through which aqueous mixtures of cells are extruded into non-aqueous fluids.

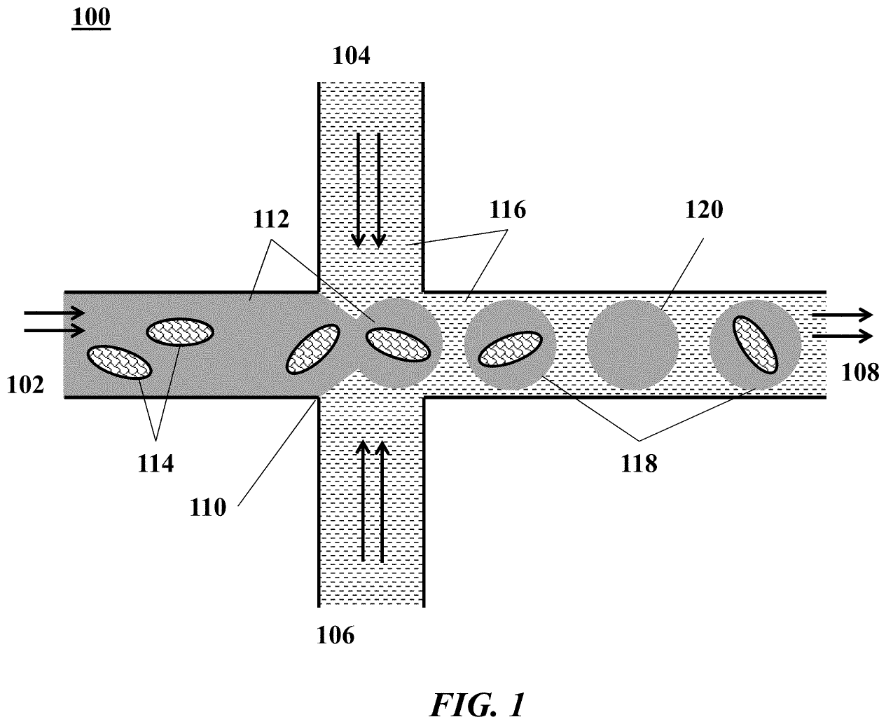

FIG. 1 shows an example of a microfluidic channel structure 100 for partitioning individual biological particles or analyte careers. The channel structure 100 can include channel segments 102, 104, 106 and 108 communicating at a channel junction 110. In operation, a first aqueous fluid 112 that includes suspended biological particles or analyte careers (e.g., cells, for example, B cells, or plasma cells) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle (e.g., cells) 114 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g., B cells, or plasma cells, and (2) unoccupied droplets 120, not containing any biological particles 114. Occupied droplets 118 can include singly occupied droplets (having one biological particle, such as one B cell or plasma cell) and multiply occupied droplets (having more than one biological particle, such as multiple B cells or plasma cells). As described elsewhere herein, in some cases, the majority of occupied partitions can include no more than one biological particle, e.g., B cells, or plasma cells, per occupied partition and some of the generated partitions can be unoccupied (of any biological particle, B cells, or plasma cells). In some cases, though, some of the occupied partitions can include more than one biological particle, e.g., B cells, or plasma cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., biological particles, such as B cells, or plasma cells 114) at the partitioning junction 110, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as B cells or plasma cells, (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

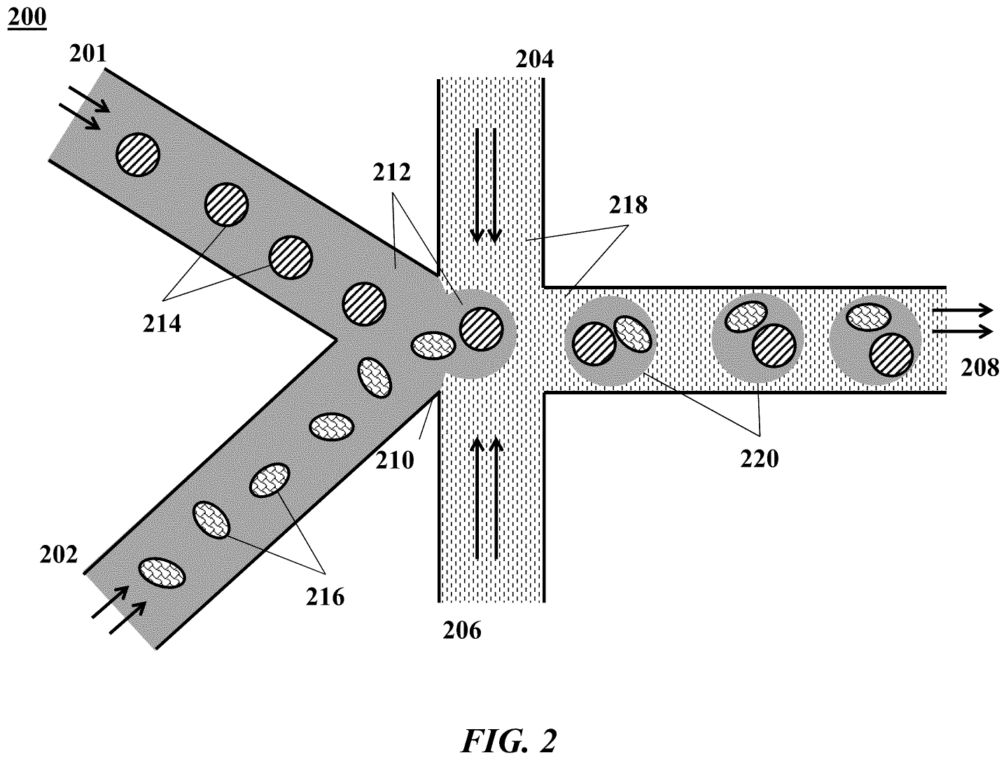

As will be appreciated, the above-described occupancy rates are also applicable to partitions that include both biological particles (e.g., cells) and additional reagents, including, but not limited to, microcapsules or beads (e.g., gel beads) carrying nucleic acid barcode molecules (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a bead comprising nucleic acid barcode molecules and a biological particle.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 can transport an aqueous fluid 212 that includes a plurality of beads 214 along the channel segment 201 into junction 210. The plurality of beads 214 can be sourced from a suspension of beads. For example, the channel segment 201 can be connected to a reservoir comprising an aqueous suspension of beads 214. The channel segment 202 can transport the aqueous fluid 212 that includes a plurality of biological particles 216 along the channel segment 202 into junction 210. The plurality of biological particles 216 can be sourced from a suspension of biological particles. For example, the channel segment 202 can be connected to a reservoir comprising an aqueous suspension of biological particles 216. In some instances, the aqueous fluid 212 in either the first channel segment 201 or the second channel segment 202, or in both segments, can include one or more reagents, as further described below.

A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 can deliver the discrete droplets to an outlet reservoir fluidly coupled to the channel segment 208, where they may be harvested. The second fluid 218 can contain an oil, such as a fluorinated oil, that includes a fluorosurfactant for stabilizing the resulting droplets, for example, inhibiting subsequent coalescence of the resulting droplets 220.

A discrete droplet that is generated can include an individual biological particle 220. A discrete droplet that is generated can include barcodes (e.g., a reporter barcode and a partition-specific barcode) or other reagent carrying bead 214. A discrete droplet generated can include both an individual biological particle and a barcode carrying bead, such as droplets 220. In some instances, a discrete droplet can include more than one individual biological particle or no biological particle. In some instances, a discrete droplet can include more than one bead or no bead. A discrete droplet can be unoccupied (e.g., no beads, no biological particles).

Beneficially, a discrete droplet partitioning a biological particle and a barcode carrying bead can effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 (FIG. 2) may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

As an alternative, the channel segments 201 and 202 can meet at another junction upstream of the junction 210. At such junction, beads and biological particles can form a mixture that is directed along another channel to the junction 210 to yield droplets 220. The mixture can provide the beads and biological particles in an alternating fashion, such that, for example, a droplet includes a single bead and a single biological particle.

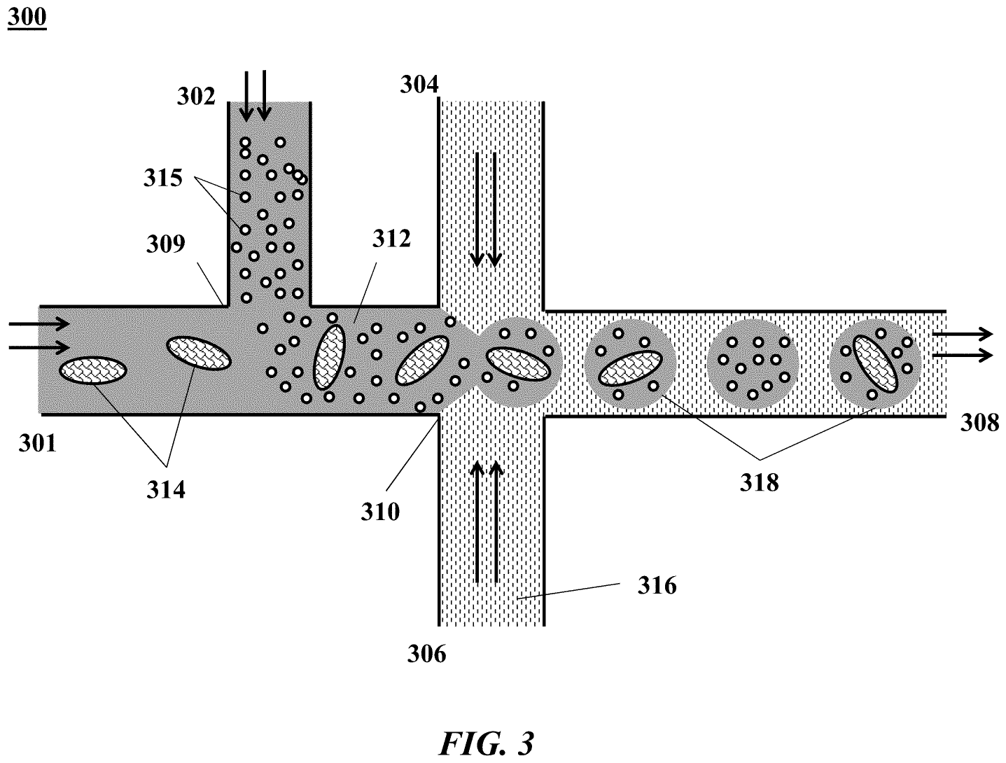

FIG. 3 shows another example of a microfluidic channel structure 300 for co-partitioning biological particles and reagents, which is described further in PCT/US2018/064600, which is hereby incorporated by reference in its entirety.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structure 300 can have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

### Controlled Partitioning

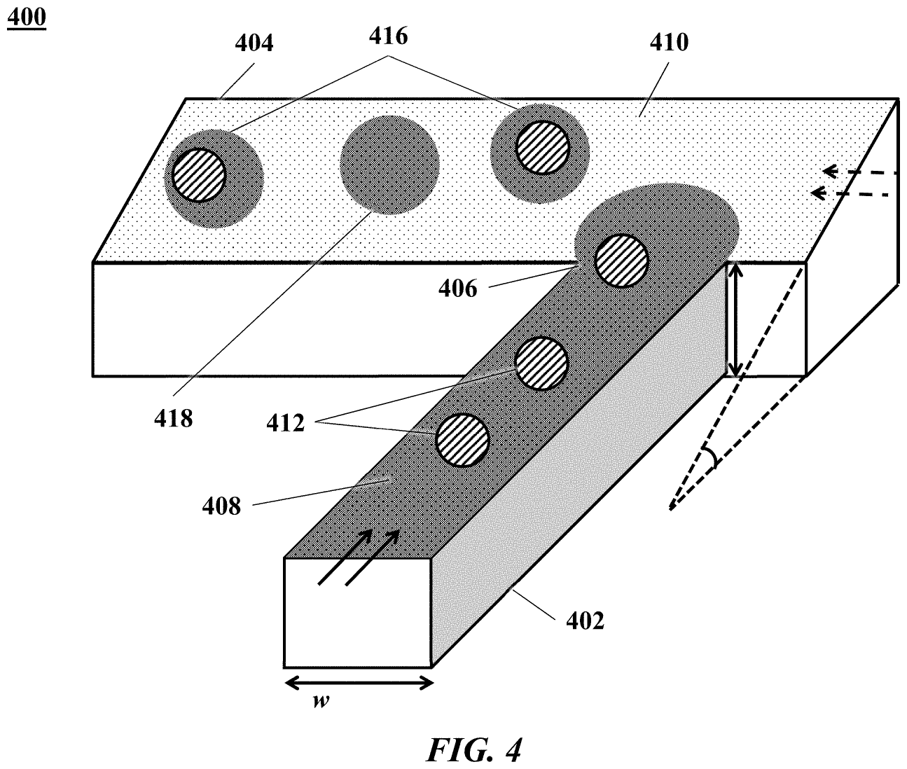

In some embodiments, provided herein are systems and methods for controlled partitioning. Droplet size can be controlled by adjusting certain geometric features in channel architecture (e.g., microfluidics channel structure). For example, an expansion angle, width, and/or length of a channel can be adjusted to control droplet size. Systems and methods for controlled partitioning are described further in PCT/US2018/047551, which is hereby incorporated by reference in its entirety. FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 can be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

A discrete droplet generated can include a bead (e.g., as in occupied droplets 416). Alternatively, a discrete droplet generated can include more than one bead. Alternatively, a discrete droplet generated may not include any beads (e.g., as in unoccupied droplet 418). In some instances, a discrete droplet generated can contain one or more biological particles, as described elsewhere herein. In some instances, a discrete droplet generated can contain one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can contain biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 can be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads can be upstream or downstream of the second separate channel introducing the biological particles.

The second fluid 410 can include an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 can be substantially stationary in the reservoir 404. In some instances, the second fluid 410 can be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 can be subjected and/or directed to flow in or out of the reservoir 404. For example, the reservoir 404 can be a channel directing the second fluid 410 from upstream to downstream, transporting the generated droplets.

The channel structure 400 at or near the junction 406 can have certain geometric features that at least partly determine the sizes of the droplets formed by the channel structure 400. The channel segment 402 can have a height, h₀ and width, w, at or near the junction 406. By way of example, the channel segment 402 can include a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, a. The expansion angle, a, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size can decrease with increasing expansion angle. The resulting droplet radius, R_{d}, can be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

The systems and methods for controlled partitioning are described further in PCT/US2018/047551, which is incorporated by reference in its entirety.

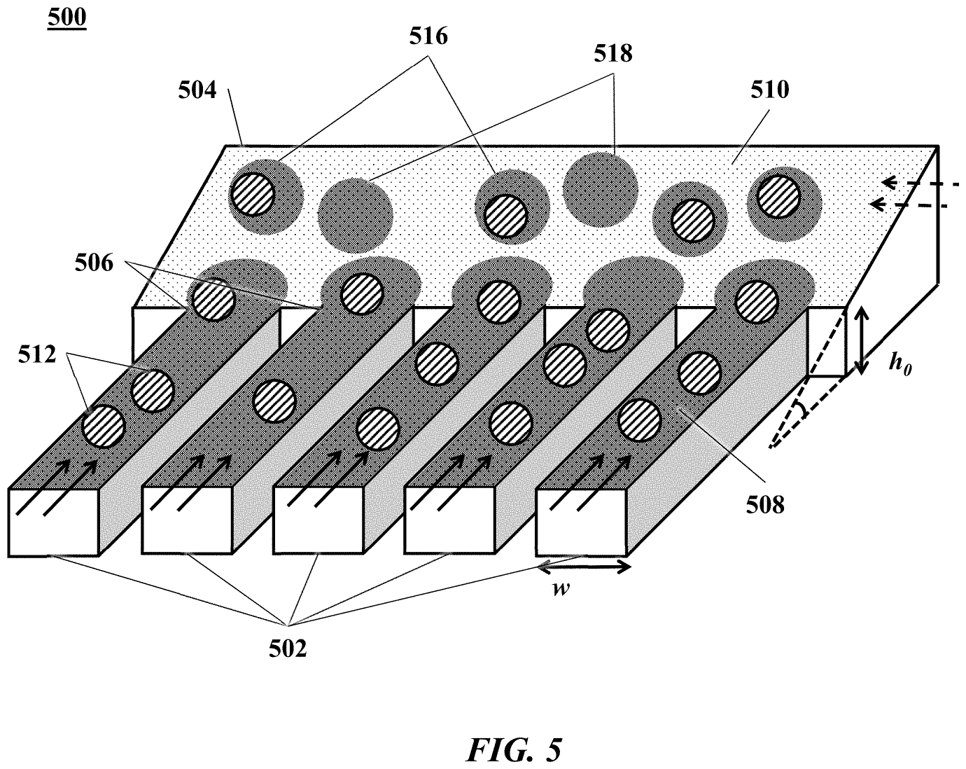

FIG. 5 shows an example of a microfluidic channel structure for increased droplet generation throughput, which is described further in PCT/US2018/064600, which is hereby incorporated by reference in its entirety.

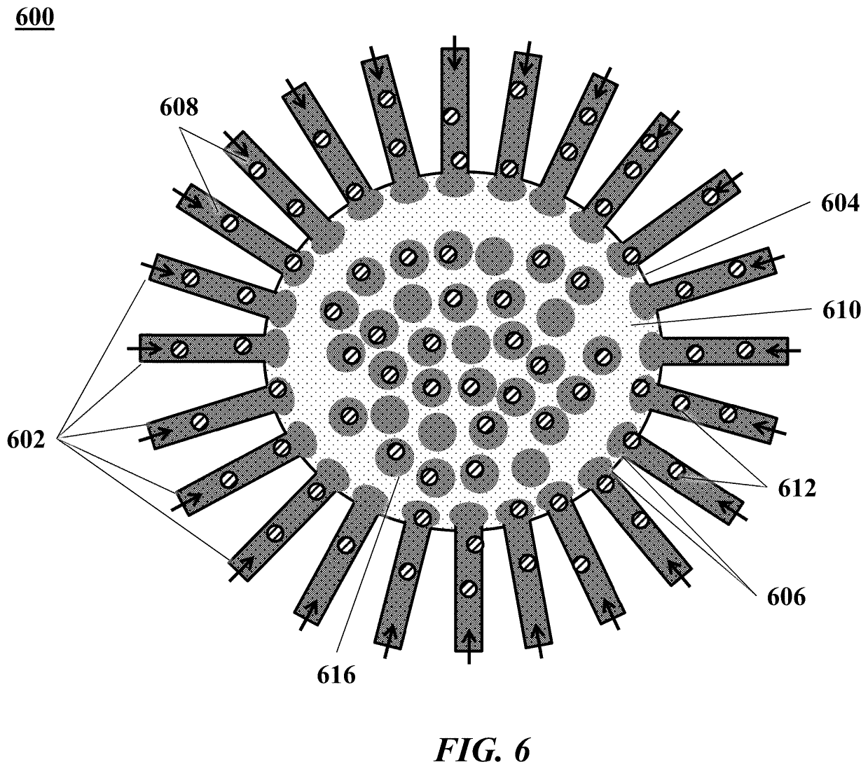

FIG. 6 shows another example of a microfluidic channel structure for increased droplet generation throughput, which is described further in PCT/US2018/064600, which is hereby incorporated by reference in its entirety.

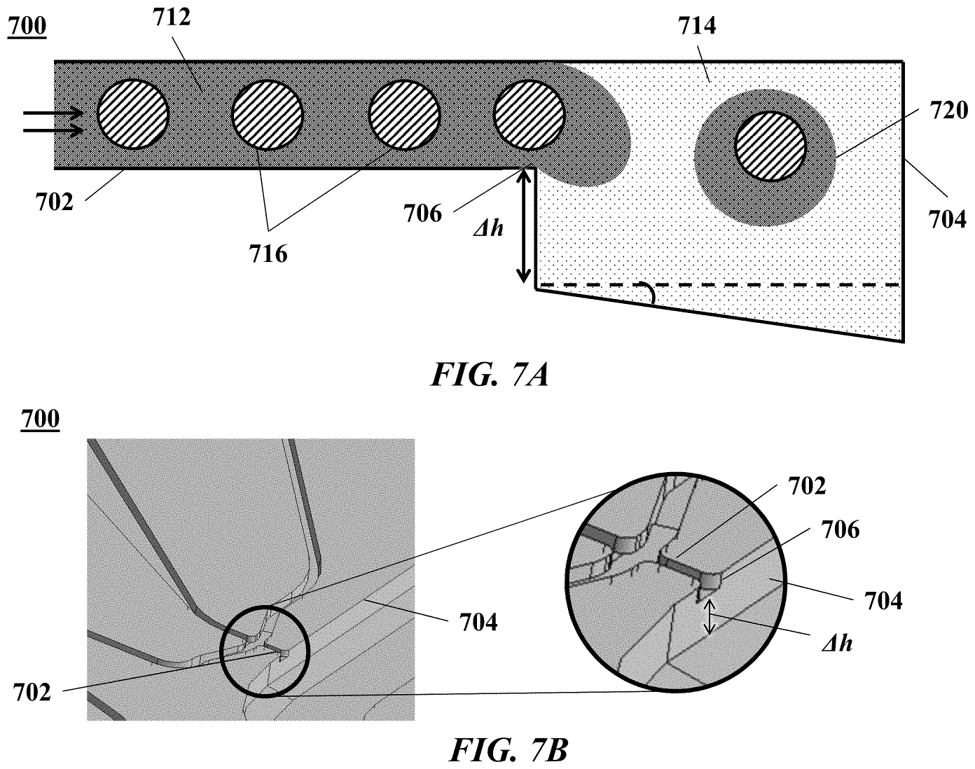

FIG. 7A shows a cross-section view of another example of a microfluidic channel structure with a geometric feature for controlled partitioning, and FIG. 7B shows a perspective view of the channel structure 700 of FIG. 7A. FIGS. 7A-B are described further in PCT/US2018/047551, which is hereby incorporated by reference in its entirety.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

### Cell Beads

In another aspect, in addition to or as an alternative to droplet-based partitioning, biological particles (e.g., cells) may be comprised within (e.g., encapsulated within) a particulate material to form a “cell bead.” In another aspect, in addition to or as an alternative to droplet-based partitioning, biological particles (e.g., cells) may be comprised within (e.g., encapsulated within) a microcapsule that comprises an outer shell, layer or porous matrix in which is entrained one or more individual biological particles or small groups of biological particles. The microcapsule may include other reagents.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively or in addition, encapsulated biological particles may be readily deposited into other partitions (e.g., droplets) as described above.

Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well can be configured to hold a volume of about 1000 μL, about 100 μL, about 10 μL, about 1 μL, about 100 nL, about 10 nL, about 1 nL, about 100 pL, about 10 pL, etc. The well can be configured to hold a volume of at least 10 pL, at least 100 pL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 pL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, etc.) while the adjacent microwell can be used to contain a cell bead, droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a cell bead or bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the cell bead or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with, cell beads, beads, or droplets. In some embodiments, any of the reagents described in this disclosure can be encapsulated in, or otherwise coupled to, a cell bead, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a cell bead, bead, or droplet that includes a set of reagents that has a similar attribute, for example, a set of enzymes, a set of minerals, a set of oligonucleotides, a mixture of different barcode molecules, a mixture of identical barcode molecules. In other cases, a cell bead, bead, or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different cell bead, droplet, or bead, or within a solution within a partition (e.g., microwell) of the system.

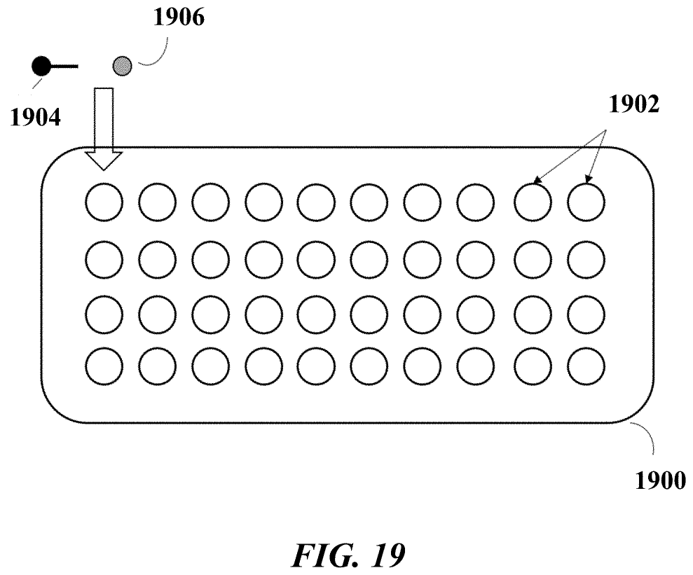

A non-limiting example of a microwell array in accordance with some embodiments of the disclosure is schematically presented in FIG. 19. In this example, the array can be contained within a substrate 1900. The substrate 1900 includes a plurality of wells 1902. The wells 1902 can be of any size or shape, and the spacing between the wells, the number of wells per substrate, as well as the density of the wells on the substrate 1900 can be modified, depending on the particular application. In one such example application, a sample molecule 1906, which can include a cell or cellular components (e.g., nucleic acid molecules) is co-partitioned with a bead 1904, which can include a nucleic acid barcode molecule coupled thereto. The wells 1902 can be loaded using gravity or other loading technique (e.g., centrifugation, liquid handler, acoustic loading, optoelectronic, etc.). In some instances, at least one of the wells 502 contains a single sample molecule 1906 (e.g., cell) and a single bead 1904.

Reagents can be loaded into a well either sequentially or concurrently. In some cases, reagents are introduced to the device either before or after a particular operation. In some cases, reagents (which can be provided, in certain instances, in cell beads, droplets, or beads) are introduced sequentially such that different reactions or operations occur at different steps. The reagents (or cell beads, droplets, or beads) can also be loaded at operations interspersed with a reaction or operation step. For example, cell beads (or droplets or beads) including reagents for fragmenting polynucleotides (e.g., restriction enzymes) and/or other enzymes (e.g., transposases, ligases, polymerases, etc.) can be loaded into the well or plurality of wells, followed by loading of cell beads, droplets, or beads including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

As described elsewhere herein, the nucleic acid barcode molecules and other reagents can be contained within a cell bead, bead, or droplet. These cell beads, beads, or droplets can be loaded into a partition (e.g., a microwell) before, after, or concurrently with the loading of a cell, such that each cell is contacted with a different cell bead, bead, or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting nucleic acid barcode molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a droplet or cell bead can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule within a sample or cell distributed in the well. For example, the nucleic acid barcode molecules can include a capture sequence that can be used to capture or hybridize to a nucleic acid molecule (e.g., RNA, DNA) within the sample. In some embodiments, the nucleic acid barcode molecules can be releasable from the microwell. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The nucleic acid barcode molecules or analytes can be further processed in the well, or the nucleic acid barcode molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) can be sealed (e.g., using an oil, membrane, wax, etc.), which enables storage of the assay or selective introduction of additional reagents

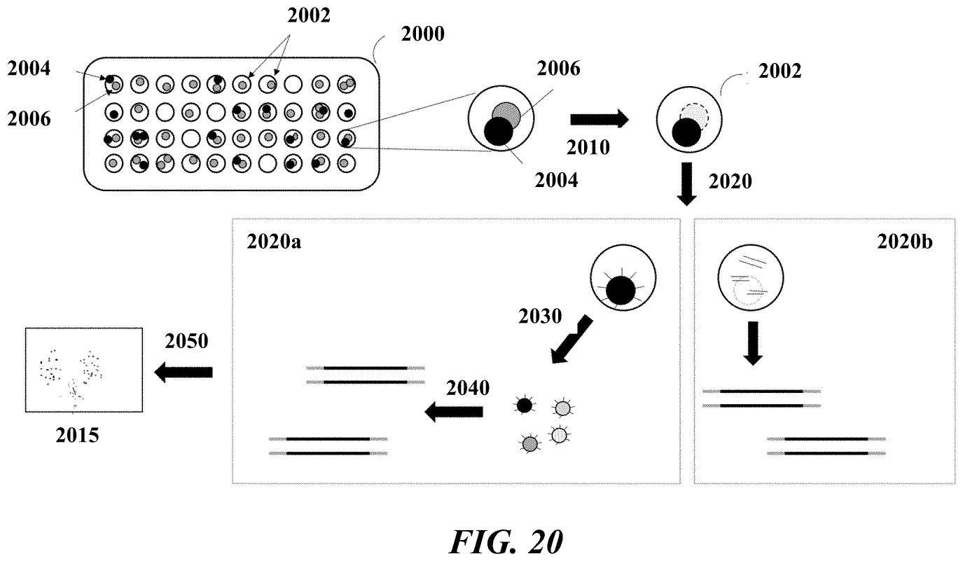

FIG. 20 schematically shows an example workflow for processing nucleic acid molecules within a sample. A substrate 2000 including a plurality of microwells 2002 can be provided. A sample 2006 which can include a cell, cell bead, cellular components or analytes (e.g., proteins and/or nucleic acid molecules) can be co-partitioned, in a plurality of microwells 2002, with a plurality of beads 2004 including nucleic acid barcode molecules. During a partitioning process, the sample 2006 can be processed within the partition. For instance, in the case of live cells, the cell can be subjected to conditions sufficient to lyse the cells and release the analytes contained therein. In process 2020, the bead 2004 can be further processed. By way of example, processes 2020a and 2020b schematically illustrate different workflows, depending on the properties of the bead 2004.

In 2020a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 2030, the beads 2004 from multiple wells 2002 can be collected and pooled. Further processing can be performed in process 2040. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 2050, further characterization, such as sequencing can be performed to generate sequencing reads. The sequencing reads can yield information on individual cells or populations of cells, which can be represented visually or graphically, e.g., in a plot.

In 2020b, the bead includes nucleic acid barcode molecules that are releasably attached thereto, as described below. The bead can degrade or otherwise release the nucleic acid barcode molecules into the well 2002; the nucleic acid barcode molecules can then be used to barcode nucleic acid molecules within the well 2002. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 2050, further characterization, such as sequencing can be performed to generate sequencing reads. The sequencing reads can yield information on individual cells or populations of cells, which can be represented visually or graphically, e.g., in a plot.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release said nucleic acid barcode molecule of said plurality of nucleic acid barcode molecules. In some cases, the labile bond comprises a disulfide linkage.

Activation of disulfide linkages within a bead can be controlled such that only a small number of disulfide linkages are activated. Methods of controlling activation of disulfide linkages within a bead are described in PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. See, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively or in addition, species may be partitioned in a partition (e.g., droplet) during or subsequent to partition formation. Such species may include, without limitation, the abovementioned species that may also be encapsulated in a bead.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### Nucleic Acid Barcode Molecules

A nucleic acid barcode molecule may contain one or more barcode sequences. A plurality of nucleic acid barcode molecules may be coupled to a bead. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid molecule can further comprise a unique molecular identifier (UMI). In some cases, the nucleic acid barcode molecule can comprise one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In some cases, the nucleic acid molecule can comprise an R1 primer sequence for Illumina sequencing. In some cases, the nucleic acid molecule can comprise an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

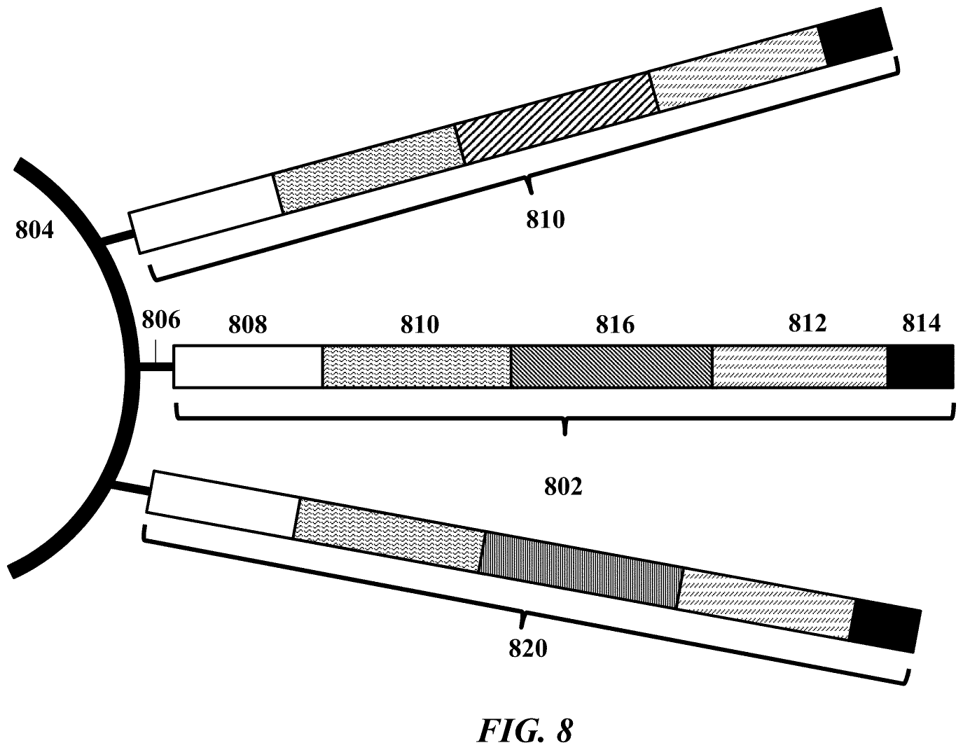

FIG. 8 illustrates an example of a barcode carrying bead. A nucleic acid barcode molecule 802 can be coupled to a bead 804 by a releasable linkage 806, such as, for example, a disulfide linker. The same bead 804 may be coupled (e.g., via releasable linkage) to one or more other nucleic acid barcode molecules 820. The nucleic acid barcode molecule 802 may be or comprise a barcode. As noted elsewhere herein, the structure of the barcode may comprise a number of sequence elements. The nucleic acid barcode molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid barcode molecules (e.g., including nucleic acid barcode molecule 802) coupled to the same bead 804. Alternatively or in addition, the barcode sequence 810 can be partition-specific such that the barcode sequence 810 is common to all nucleic acid barcode molecules coupled to one or more beads that are partitioned into the same partition. The nucleic acid barcode molecule 802 may comprise sequence 812 complementary to an analyte of interest, e.g., a priming sequence. Sequence 812 can be a poly-T sequence complementary to a poly-A tail of an mRNA analyte, a targeted priming sequence, and/or a random priming sequence. The nucleic acid barcode molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid barcode molecules (e.g., 802, 810, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 8 shows three nucleic acid barcode molecules 802, 810, 820 coupled to the surface of the bead 304, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 808, 810, 812, etc.) and variable or unique sequence segments (e.g., 816) between different individual nucleic acid barcode molecules coupled to the same bead.

In operation, an biological particle (e.g., cell, DNA, RNA, etc.) can be co-partitioned along with a barcode bearing bead 804. The nucleic acid barcode molecules 802, 810, 820 can be released from the bead 804 in the partition. By way of example, in the context of analyzing sample RNA, the poly-T segment (e.g., 812) of one of the released nucleic acid barcode molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid barcode molecule 802. Because the nucleic acid barcode molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

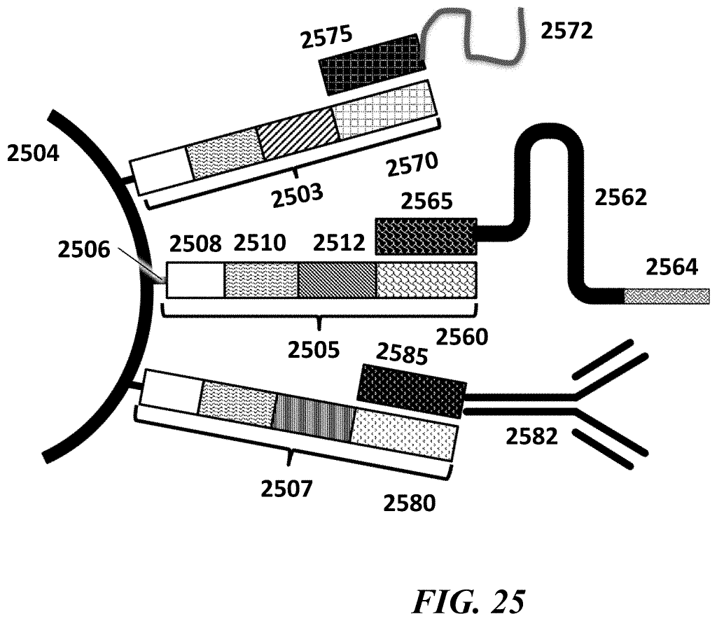

FIG. 25 illustrates another example of a barcode carrying bead. A nucleic acid barcode molecule 2505, such as an oligonucleotide, can be coupled to a bead 2504 by a releasable linkage 2506, such as, for example, a disulfide linker. The nucleic acid barcode molecule 2505 may comprise a first capture sequence 2560. The same bead 2504 may be coupled (e.g., via releasable linkage) to one or more other nucleic acid molecules 2503, 2507 comprising other capture sequences. The nucleic acid barcode molecule 2505 may be or comprise a barcode. As noted elsewhere herein, the structure of the barcode may comprise a number of sequence elements, such as a functional sequence 2508 (e.g., flow cell attachment sequence, sequencing primer sequence, etc.), a barcode sequence 2510 (e.g., bead-specific sequence common to bead, partition-specific sequence common to partition, etc.), and a unique molecular identifier 2512 (e.g., unique sequence within different molecules attached to the bead), or partial sequences thereof. The capture sequence 2560 may be configured to attach to a corresponding capture sequence 2565. In some instances, the corresponding capture sequence 2565 may be coupled to another molecule that may be an analyte or an intermediary carrier. For example, as illustrated in FIG. 25, the corresponding capture sequence 2565 is coupled to a guide RNA molecule 2562 comprising a target sequence 2564, wherein the target sequence 2564 is configured to attach to the analyte. Another oligonucleotide molecule 2507 attached to the bead 2504 comprises a second capture sequence 2580 which is configured to attach to a second corresponding capture sequence 2585. As illustrated in FIG. 25, the second corresponding capture sequence 2585 is coupled to an antibody 2582. In some cases, the antibody 2582 may have binding specificity to an analyte (e.g., surface protein). Alternatively, the antibody 2582 may not have binding specificity. Another oligonucleotide molecule 2503 attached to the bead 2504 comprises a third capture sequence 2570 which is configured to attach to a second corresponding capture sequence 2575. As illustrated in FIG. 25, the third corresponding capture sequence 2575 is coupled to a molecule 2572. The molecule 2572 may or may not be configured to target an analyte. The other oligonucleotide molecules 2503, 2507 may comprise the other sequences (e.g., functional sequence, barcode sequence, UMI, etc.) described with respect to oligonucleotide molecule 2505. While a single oligonucleotide molecule comprising each capture sequence is illustrated in FIG. 25, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 2504 may comprise other capture sequences. Alternatively or in addition, the bead 2504 may comprise fewer types of capture sequences (e.g., two capture sequences). Alternatively or in addition, the bead 2504 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads, such as beads, are provided that each include large numbers of the above described nucleic acid barcode molecules releasably attached to the beads, where all of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

### Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more biological particles, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate biological particles of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS).

Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles or analyte careers described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned cell bead. For example, in some cases, a chemical stimulus may be co-partitioned along with an encapsulated biological particles to allow for the degradation of the cell bead and release of the cell or its contents into the larger partition. In some cases, this stimulus may be the same as the stimulus described elsewhere herein for release of nucleic acid molecules (e.g., partition-specific molecules) from their respective cell bead. In alternative examples, this may be a different and non-overlapping stimulus, in order to allow an encapsulated biological particles to be released into a partition at a different time from the release of nucleic acid molecules (e.g., partition-specific barcode molecules) into the same partition. For a description of methods, compositions, and systems for encapsulating cells (also referred to as a “cell bead”), see, e.g., U.S. Pat. No. 10,428,326 and U.S. Pub. 20190100632, which are each incorporated by reference in their entirety.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. Template switching is further described in PCT/US2017/068320, which is hereby incorporated by reference in its entirety. Template switching oligonucleotides may comprise a hybridization region and a template region. Template switching oligonucleotides are further described in PCT/US2017/068320, which is hereby incorporated by reference in its entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations.

### Multiplexing Methods

The present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labeling agents capable of binding to or otherwise coupling to one or more cells or cell features can be used to characterize cells and/or cell features. In some instances, cell features include cell surface features. Cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labeling agent can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labeling agents can include (e.g., are attached to) a reporter oligonucleotide that is indicative of the cell surface feature to which the binding group binds. For example, the reporter oligonucleotide can include a barcode sequence that permits identification of the labeling agent. For example, a labeling agent that is specific to one type of cell feature (e.g., a first cell surface feature) can have a first reporter oligonucleotide coupled thereto, while a labeling agent that is specific to a different cell feature (e.g., a second cell surface feature) can have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labeling agents can be provided, where the respective cell feature labeling agents are associated with nucleic acid reporter molecules, such that a different reporter oligonucleotide sequence is associated with each labeling agent capable of binding to a specific cell feature. In other aspects, different members of the library can be characterized by the presence of a different oligonucleotide sequence label. For example, an antibody capable of binding to a first protein can have associated with it a first reporter oligonucleotide sequence, while an antibody capable of binding to a second protein can have a different reporter oligonucleotide sequence associated with it. The presence of the particular oligonucleotide sequence can be indicative of the presence of a particular antibody or cell feature which can be recognized or bound by the particular antibody.

Labeling agents capable of binding to or otherwise coupling to one or more cells can be used to characterize a cell as belonging to a particular set of cells. For example, labeling agents can be used to label a sample of cells or a group of cells. In this way, a group of cells can be labeled as different from another group of cells. In an example, a first group of cells can originate from a first sample and a second group of cells can originate from a second sample. Labeling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM.

In another example, a reporter oligonucleotide can be coupled to a cell-penetrating peptide (CPP), and labeling cells can include delivering the CPP coupled reporter oligonucleotide into an biological particle. Labeling biological particles can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600, which is hereby incorporated by reference in its entirety.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells can be incubated with the library of labeling agents, that can be labeling agents to a broad panel of different cell features, e.g., receptors, proteins, etc., and which include their associated reporter oligonucleotides. Unbound labeling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labeling agents and their known, associated reporter oligonucleotides.

In other instances, e.g., to facilitate sample multiplexing, a labeling agent that is specific to a particular cell feature can have a first plurality of the labeling agent (e.g., an antibody or lipophilic moiety) coupled to a first reporter oligonucleotide and a second plurality of the labeling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

In some embodiments, to facilitate sample multiplexing, individual samples can be stained with lipid tags, such as cholesterol-modified oligonucleotides (CMOs, see, e.g., FIG. 13), anti-calcium channel antibodies, or anti-ACTB antibodies. Non-limiting examples of anti-calcium channel antibodies include anti-KCNN4 antibodies, anti-BK channel beta 3 antibodies, anti-a1B calcium channel antibodies, and anti-CACNA1A antibodies. Examples of anti-ACTB antibodies suitable for the methods of the disclosure include, but are not limited to, mAbGEa, ACTN05, AC-15, 15G5A11/E2, BA3R, and HHF35.

As described elsewhere herein, libraries of labeling agents can be associated with a particular cell feature as well as be used to identify analytes as originating from a particular cell population, or sample. Cell populations can be incubated with a plurality of libraries such that a cell or cells include multiple labeling agents. For example, a cell can include coupled thereto a lipophilic labeling agent and an antibody. The lipophilic labeling agent can indicate that the cell is a member of a particular cell sample, whereas the antibody can indicate that the cell includes a particular analyte. In this manner, the reporter oligonucleotides and labeling agents can allow multi-analyte, multiplexed analyses to be performed.

In some instances, these reporter oligonucleotides can include nucleic acid barcode sequences that permit identification of the labeling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labeling agents can be achieved through any of a variety of direct or indirect, covalent or non-covalent associations or attachments. For example, oligonucleotides can be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, can be used to couple reporter oligonucleotides to labeling agents. Commercially available kits, such as those from Thunderlink and Abcam, and techniques common in the art can be used to couple reporter oligonucleotides to labeling agents as appropriate. In another example, a labeling agent is indirectly (e.g., via hybridization) coupled to a reporter oligonucleotide including a barcode sequence that identifies the label agent. For instance, the labeling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labeling agent can include a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

In some instances, analysis of multiple analytes (e.g., nucleic acids and one or more analytes using labeling agents described herein) can be performed. For example, the workflow can include a workflow as generally depicted in any of FIGS. 12A-12C, or a combination of workflows for an individual analyte, as described elsewhere herein. For example, by using a combination of the workflows as generally depicted in FIGS. 12A-12C, multiple analytes can be analyzed.

Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

### Computer Systems

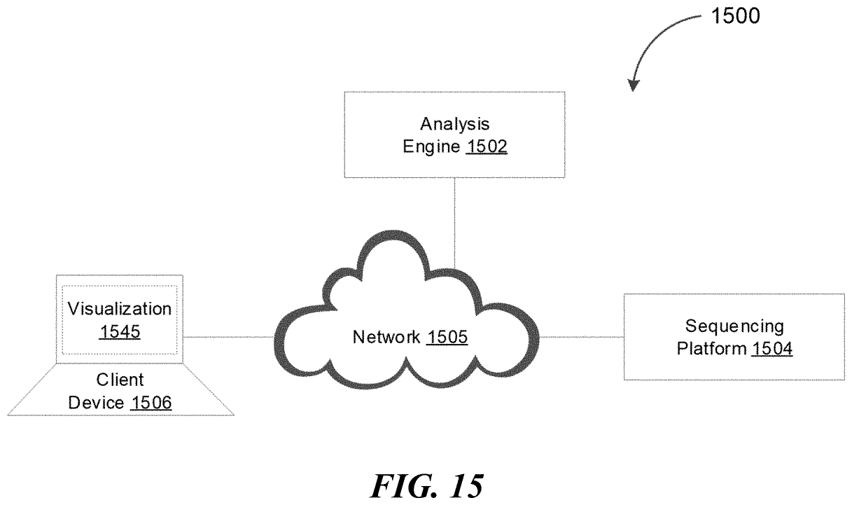

The present disclosure also provides computer systems configured to implement the various methods disclosed herein including, for example, methods to identify an antigen binding molecule, quantify a subject's response to an antigen, determine a diversity of a subject's immune response to an antigen, monitor a subject's response to an antibody therapeutic or antibody drug conjugate (ADC), characterize cells expressing one or more antigen binding molecules that bind to an antigen, identify an antibody therapeutic that does not elicit an immune response, and/or the like. FIG. 15 depicts a system diagram illustrating an example of an analysis system 1500, in accordance with some example embodiments. Referring to FIG. 15, the analysis system 1500 may include an analysis engine 1502, a sequencing platform 1504, and a client device 1506. As shown in FIG. 15, the analysis engine 1502, the sequencing platform 1504, and the client device 1506 may be communicatively coupled via a network 1505. The network 1505 may be a wired network and/or a wireless network including, for example, a local area network (LAN), a virtual local area network (VLAN), a wide area network (WAN), a public land mobile network (PLMN), the Internet, and/or the like.

Referring again to FIG. 15, in some example embodiments, the analysis engine 1502 may receive, from the sequencing platform 1504, data associated with a nucleic acid barcode molecule containing sequence information from a partition-specific barcode molecule and a reporter oligonucleotide conjugated to each of a plurality of antigens. As noted, the reporter oligonucleotide conjugated to an antigen may include a sequence, such as a reporter barcode, that enables an identification of the antigen. Conjugating each of the plurality of antigens with a reporter oligonucleotide that includes a reporter barcode may further enable a differentiation between different antigens, for example, during a multiplexed antigen assay. To further facilitate the processing and identification of the reporter barcode (e.g., through nucleic acid sequencing), the reporter oligonucleotide may be coupled with a partition-specific barcode molecule that includes one or more of a partition-specific barcode, and a template switching oligonucleotide (TSO) site.

In some example embodiments, the analysis engine 1502 may generate, based at least on the data received from the sequencing platform 1504, a count matrix indicating a count of a quantity of times each of the plurality of antigens bound to each of a plurality of cells expressing one or more antigen binding molecules. In some embodiments, the count matrix contains information relating to, but not limited to, surface protein (non-antigen), intracellular protein (non-antigen), gene expression (targeted or untargeted RNA), DNA accessibility/ATAC, metabolite data, CRISPR guide, and/or DNA repair activity. To further illustrate, FIG. 16 depicts an example of a count matrix 1600, in accordance with some example embodiment (see Example 10). Referring to FIG. 16, each row of the count matrix 1600 may correspond to a cell expressing an antigen binding molecule (e.g., mAb₁, mAb₂, and/or the like) while each column of the count matrix 1600 may correspond to an antigen (e.g., Ag₁, Ag₂, Ag₃, Ag₄, Ag₅, and/or the like). Accordingly, each element of the count matrix 1600 may correspond to a count of a quantity of times an antigen bound to a cell expressing an antigen binding molecule. For example, according to the example of the count matrix 1600 shown in FIG. 16, indicates that a first antigen Ag₁ bound to a first cell expressing an antigen binding molecule mAb₁ 347 times while the first antigen Ag₁ bound to a second cell expressing an antigen binding module mAb₂ 21 times. In some embodiments, the antigen binding molecule is a secreted antibody. In some embodiments, the antigen binding molecule is a surface-bound antibody. FIG. 22 depicts another example of a count matrix 2200, in accordance with some example embodiment (see Example 11). Referring to FIG. 22, each row of the count matrix 2200 may correspond to a cell expressing an antibody therapeutic (e.g., AbTx 1 (unmodified), AbTx 2 (containing an engineered mutation X), and AbTx 3 (containing an engineered mutation Y), while each column of the count matrix may correspond to an anti-drug antibody (ADA) identified according to the methods disclosed herein (e.g., ADA-1, ADA-2, ADA-3, and/or the like). Accordingly, each element of the count matrix may correspond to a count of a quantity of times an ADA bound to a cell expressing a particular AbTx. For example, according to the example of the count matrix shown in FIG. 22, indicates that a first ADA (ADA-1) bound to a first cell expressing AbTx 1 347 times while the ADA (ADA-1) bound to a second cell expressing AbTx 2 21 times. FIG. 23 depicts yet another example of a count matrix 2300, in accordance with some example embodiment (see Example 12). Referring to FIG. 23, each row of the count matrix 2300 may correspond to a cell expressing an antibody therapeutic (e.g., AbTx 1 (unmodified), AbTx 2 (containing an engineered mutation X), and AbTx 3 (containing an engineered mutation Y), while each column of the count matrix may correspond to an anti-drug antibody (ADA) identified according to the methods disclosed herein (e.g., ADA-1, ADA-2, ADA-3, and/or the like, and a ligand A). Accordingly, each element of the count matrix may correspond to a count of a quantity of times an ADA bound to a cell expressing a particular AbTx. For example, according to the example of the count matrix shown in FIG. 23, indicates that a first ADA (ADA-1) bound to a first cell expressing AbTx 1 347 times while the ADA (ADA-1) bound to a second cell expressing AbTx 2 21 times.

In some example embodiments, the analysis engine 1502 may embed the count matrix, for example, the count matrix 1600, in a lower dimensional space in order to identify one or more distinct populations of cells expressing an antigen binding molecule capable of binding to one or more of a same antigen. The count matrix may provide high dimensional raw data as each cell expressing an antigen many be capable of binding to many different antigens, each of antigen adding to the dimensionality of the raw data. The high dimensionality of the data associated with the count matrix may obscure the similarity between populations of cells that are capable of binding to one or more of the same antigens. Embedding the count matrix in the lower dimensional space, which reduces the dimensionality of the data associated with the count matrix, may therefore enable the identification of one or more distinct populations of cells expressing an antigen binding molecule capable of binding to one or more of a same antigen.

In some example embodiments, the analysis engine 1502 may preprocess the count matrix prior to generating a reduced dimension representation of the count matrix. For example, the analysis engine 1502 may preprocess the count matrix by applying various techniques such as filtering, transforming, and/or denoising the data associated with the count matrix. The preprocessing of the count matrix may minimize the sources of unwanted variations in the data associated with the count matrix. Preprocessing the count matrix may therefore improve the performance of subsequent dimensionality reduction techniques such as, for example, principal component analysis (PCA), uniform manifold approximation and projection (UMAP), T-distributed Stochastic Neighbor Embedding (t-SNE), Poincare disks, and/or the like.

In some example embodiments, the analysis engine 1502 may preprocess the count matrix by at least filtering the count matrix to retain one or more cells expressing one or more antigen binding molecules having a detected variability, diversity, and joining (VDJ) sequence and/or an antibody sequence, a non-zero binding count, and a sufficient sequencing depth (e.g., 5,000 to 20,000 reads per cell for antigen and VDJ libraries).

Furthermore, the analysis engine 1502 may preprocess the count matrix, for example, the filtered count matrix, by calculating an ambient concentration (e.g., in mean, median, hypergeometric mean, and/or the like) of one or more antigens and/or the reporter oligonucleotides conjugated to the antigens as well as an deviation for non-cell-associated (e.g., empty) barcodes. The count matrix may be further preprocessed by applying a transformation, adding a pseudocount, subtracting the ambient concentration, and scaling. Examples of transformations may include variance stabilizing transformation, square root transformation, cubic root transformation, and log transformation. The analysis engine 1502 may apply the transformation to each element that is included in the filtered count matrix before adding a pseudocount (e.g., ranging from 1 to 30), subtracting the ambient concentration, and scaling (e.g., dividing) by the deviation or variance of the corresponding antigen. The analysis engine 1502 may further modify the count matrix by fitting the count vector for each cell to a mixture model (e.g., a Gaussian mixture model) including one or more covariates such as a quantity of detected reads, a quantity of detected genes, a quantity of detected gene unique molecular identifiers (UMIs), a quantity of detected antigens, a quantity of detected antigen unique molecular identifiers (UMIs), a quantity of detected surface or intracellular protein unique molecular identifiers, a quantity of detected ATAC peaks, a quantity of detected surface proteins, a quantity of detected intracellular proteins, B-cell phenotypes, an IgG constant region, a quantity of mutations in antibody sequence, sequencing depth, quantity of detected unique molecular identifiers (UMIs), cell annotations, and/or the like.

To further illustrate, referring again to FIG. 16 and the example of the count matrix 1600, once the count matrix 1600 is filtered to retain, for example, at least the first cell mAb₁ and the second cell mAb₂, the analysis engine 1502 may further preprocess the count matrix 1600 by calculating an ambient concentration of each of the first antigen Ag₁, the second antigen Ag₂, the third antigen Ag₃, the fourth antigen Ag₄, the fifth antigen Ag₅, and/or the like. The analysis engine 1502 may further transform each element in the count matrix 1600 before adding a pseudocount (e.g., ranging from 1 to 30), subtracting the ambient concentration, and scaling (e.g., dividing) by the deviation or variance of the corresponding antigen. For example, the analysis engine 1502 may transform the count indicating the first antigen Ag₁ bound to the first cell mAb₁ 347 times by applying a variance stabilizing transformation, a square root transformation, a cubic root transformation, a log transformation, and/or the like. The analysis engine 1502 may add a pseudocount (e.g., ranging from 1 to 30), subtract the ambient concentration (e.g., of the first antigen Ag₁), and scale (e.g., divide) by the deviation or variance of the first antigen Ag₁. Moreover, the analysis engine 1502 may further modify the count matrix 1600 by fitting the count vector associated with each of the first cell mAb₁ and the second cell mAb₂, which includes the quantity of times each cell bound to each of the antigens Ag₁, Ag₂, Ag₃, Ag₄, Ag₅, to a mixture model (e.g. a Gaussian mixture model and/or the like) that includes various covariates such as a quantity of detected reads, a quantity of detected genes, a quantity of detected gene unique molecular identifiers (UMIs), a quantity of detected antigens, a quantity of detected antigen unique molecular identifiers (UMIs), a quantity of detected surface or intracellular protein unique molecular identifiers, a quantity of detected ATAC peaks, a quantity of detected surface proteins, a quantity of detected intracellular proteins, B-cell phenotypes, an IgG constant region, a quantity of mutations in antibody sequence, sequencing depth, quantity of detected unique molecular identifiers (UMIs), cell annotations, and/or the like.

Alternatively, the analysis engine 1502 may preprocess the count matrix, for example, the filtered count matrix, by calculating an ambient concentration (e.g., in mean, median, hypergeometric mean, and/or the like) of one or more antigens and/or the reporter oligonucleotides conjugated to the antigens before subtracting the ambient concentration from each antigen included in the count matrix (e.g., antigens Ag₁, Ag₂, Ag₃, Ag₄, and Ag₅ in the count matrix 1600). The analysis engine 1502 may, optionally, add a pseudocount (e.g., ranging from 1 to 50) before applying a transformation such as, for example, a variance stabilizing transformation, a square root transformation, a cubic root transformation, a log transformation, and/or the like.

In some example embodiments, the analysis engine 1502 may generate a reduced dimension representation of the count matrix by applying, to the preprocessed count matrix, one or more dimensionality reduction techniques such as, for example, principal component analysis (PCA), uniform manifold approximation and projection (UMAP), T-distributed Stochastic Neighbor Embedding (t-SNE), Poincare disks, and/or the like. Generating the reduced dimension representation of the count matrix may enable the analysis engine 1502 to generate, for example, for display at the client device 1506, a corresponding visualization 1545 of the count matrix and/or the populations of cells included in the count matrix. Further application of a clustering technique (e.g., spectral clustering and/or the like) or a community detection algorithm (e.g., granularity-based community detection, resolution-based community detection, and/or the like) may enable the analysis engine 1502 to generate the visualization 1545 to depict groups of cells that have been separated into distinct populations of cells having a similar binding profile (e.g., capable of binding to one or more of a same antigen).

FIG. 17 depicts an example of the visualization 1545, which may be presented as part of a graphic user interface (GUI) at the client device 1506. The example of the visualization 1545 may be generated based on the example of the count matrix 1600 shown in FIG. 16. For example, the visualization 1545 may be generated based on a reduced dimension representation of the count matrix 1600 generated by applying, to the count matrix 1600, one or more of a principal component analysis (PCA), a uniform manifold approximation and projection (UMAP), a T-distributed Stochastic Neighbor Embedding (t-SNE), Poincare disks, and/or the like. The visualization 1545 may be further generated by applying a clustering technique (e.g., spectral clustering and/or the like) or a community detection algorithm (e.g., granularity based community detection, resolution based community detection). Accordingly, the visualization 1545 may depict distinct populations of cells having a similar binding profile (e.g., capable of binding to one or more of a same antigen) including, for example, a first population of cells 1702 having a similar binding profile as the first cell mAb₁ and a second population of cells 1704 having a similar binding profile as the second cell mAb₂.

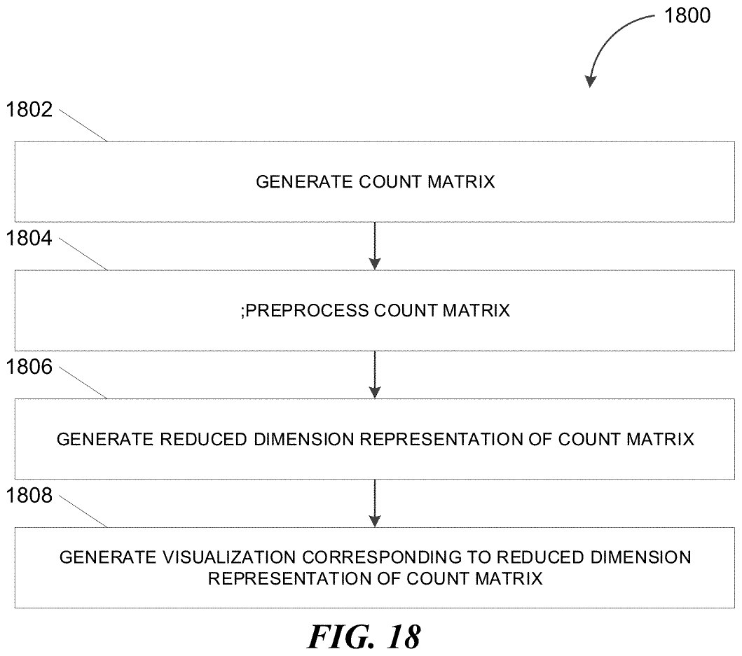

FIG. 18 depicts a flowchart illustrating an example of a process 1800 for analyzing and visualizing data associated with cells expressing one or more antigen binding molecules, in accordance with some example embodiments. Referring to FIGS. 15 and 18, the process 1800 may be performed by the analysis engine 1502 to generate, analyze, and visualize, for example, the count matrix 1600, which may include data indicating a quantity of times one or more cells expressing an antigen binding molecule (e.g., the first cell mAb₁, the second cell mAb₂, and/or the like) bound to an antigen (e.g., Ag₁, Ag₂, Ag₃, Ag₄, Ag₅, and/or the like).

At 1802, the analysis engine 1502 may generate a count matrix. For example, the analysis engine 1502 may receive, from the sequencing platform 1504, data associated with a nucleic acid barcode molecule containing sequence information from a partition-specific barcode molecule and a reporter oligonucleotide conjugated to each of a plurality of antigens. The reporter oligonucleotide conjugated to an antigen may include a unique sequence, such as a reporter barcode, that enables an identification of the antigen as well as a differentiation between different antigens. As such, the analysis engine 1502 may generate, based at least on the data received form the sequencing platform 1504, a count matrix indicating a count of a quantity of times each of the plurality of antigens bound to each of a plurality of cells expressing one or more antigen binding molecules. For instance, in the example of the count matrix 1600 shown in FIG. 16, the first antigen Ag₁ bound to the first cell mAb₁ 347 times and to the second cell mAb₂ 21 times.

At 1804, the analysis engine 1502 may preprocess the count matrix. In some example embodiments, the analysis engine 1502 may, prior to generating a reduced dimension representation of the count matrix, preprocess the count matrix by applying various techniques such as such as filtering, transforming, and/or denoising the data associated with the count matrix. The analysis engine 1502 may preprocess the count matrix by at least filtering the count matrix to retain one or more cells expressing one or more antigen binding molecules having a detected variability, diversity, and joining (VDJ) sequence and/or an antibody sequence, a non-zero binding count, and a sufficient sequencing depth (e.g., 5,000 to 20,000 reads per cell for antigen and VDJ libraries). Moreover, the analysis engine 1502 may preprocess the count matrix, for example, the filtered count matrix, by calculating an ambient concentration (e.g., in mean, median, hypergeometric mean, and/or the like) of one or more antigens and/or the reporter oligonucleotides conjugated to the antigens. The analysis engine 1502 may apply a transformation (e.g., a variance stabilizing transformation, a square root transformation, a cubic root transformation, a log transformation, and/or the like) to each cell that is included in the filtered count matrix, add a pseudocount (e.g., ranging from 1 to 30), subtract the ambient concentration, and/or scale (e.g., divide) by the deviation or variance of the corresponding antigen. Alternatively and/or additionally, the analysis engine 1502 may modify the count matrix by fitting the count vector for each cell to a mixture model (e.g., a Gaussian mixture model) including one or more covariates such as a quantity of detected reads, a quantity of detected genes, a quantity of detected gene unique molecular identifiers (UMIs), a quantity of detected antigens, a quantity of detected antigen unique molecular identifiers (UMIs), a quantity of detected surface or intracellular protein unique molecular identifiers, a quantity of detected ATAC peaks, a quantity of detected surface proteins, a quantity of detected intracellular proteins, B-cell phenotypes, an IgG constant region, a quantity of mutations in antibody sequence, sequencing depth, quantity of detected unique molecular identifiers (UMIs), cell annotations, and/or the like.

At 1806, the analysis engine 1502 may generate a reduced dimension representation of the count matrix. For example, the analysis engine 1502 may generate a reduced dimension representation of the count matrix by applying, to the preprocessed count matrix, one or more dimensionality reduction techniques such as principal component analysis (PCA), uniform manifold approximation and projection (UMAP), T-distributed Stochastic Neighbor Embedding (t-SNE), Poincare disks, and/or the like. As noted, the high dimensionality of the raw data associated with the count matrix may obscure the similarity between populations of cells that are capable of binding to one or more of the same antigens. Reducing the dimensionality of the data associated with the count matrix, for example, by generating a corresponding reduced dimension representation, may therefore enable the identification of one or more distinct populations of cells expressing an antigen binding molecule capable of binding to one or more of a same antigen.

At 1808, the analysis engine 1502 may generate a visualization corresponding to the reduced dimension representation of the count matrix. For example, the analysis engine 1502 may generate, based on the reduced dimension representation of the count matrix, the visualization 1545 to depict the count matrix and/or the populations of cells included in the count matrix. By applying a clustering technique (e.g., spectral clustering and/or the like) or a community detection algorithm (e.g., granularity based community detection, resolution based community detection), the analysis engine 1502 may further generate the visualization 1545 to depict groups of cells having a similar binding profile (e.g., capable of binding to one or more of a same antigen). For instance, the example of the visualization 1545 shown in FIG. 17 may depict the first population of cells 1702 having a similar binding profile as the first cell mAb₁ and the second population of cells 1704 having a similar binding profile as the second cell mAb₂. The visualization 1545 may be presented as part of the graphic user interface (GUI) at the client device 1506.

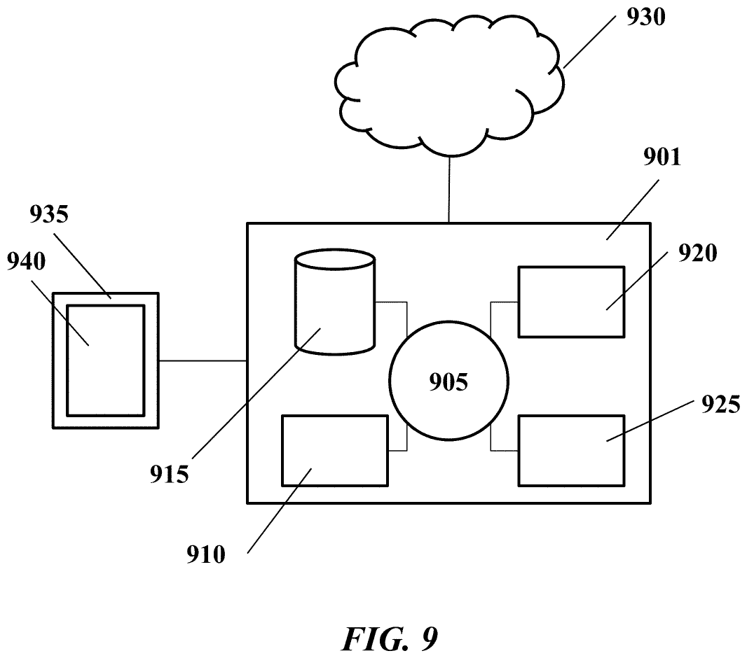

FIG. 9 depicts a block diagram illustrating an example of a computer system 901, in accordance with some example embodiments. Referring to FIGS. 9 and 15, the computer system 901 may be configured to implement one or more of the analysis engine 1502, the sequencing platform 1504, and the client device 1506. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that includes a user interface (UI) 940 for providing, for example, results of the assay, such as a summary of one or more antigen binding molecules that bind the antigens, a summary of one or more antigens not bound by an antigen binding molecule in the composition, a site on the antigen that binds to the antigen binding molecule, or proposed modifications to the antigen that can reduce affinity of the antigen binding fragment for the antigen. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, contact an antigen with an antigen binding molecule, isolate the antigen binding molecule, or identify the antigen binding molecule as described herein. In some embodiments, an algorithm can determine a site on the antigen binding to the antigen binding molecule, such as an epitope. In some embodiments, an algorithm can determine a modification of an antigen or to modify an antigen to reduce affinity of the antigen binding molecule for the antigen.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1: Identification of Mutations in Therapeutic Antibodies or Antibody-Drug Conjugates

One or more mutations in therapeutic antibodies or antibody-drug conjugates that eliminate the recognition of the mutated therapeutic antibodies or antibody-drug conjugates comprising such mutations by the preexisting anti-drug antibodies can be determined by methods provided herein.

A count matrix of cells and drug-reactive antibodies conjugated to reporter oligonucleotides is derived. In some embodiments, a count matrix of cells expressing drug-reactive antibodies, and therapeutic antibodies conjugated to reporter oligonucleotides is derived. In some embodiments, a count matrix of cells expressing unmodified or mutated therapeutic antibodies and drug-reactive antibodies conjugated to reporter oligonucleotides is derived. In some embodiments, a count matrix of cells expressing drug-reactive antibodies, therapeutic antibodies conjugated to reporter oligonucleotides, and therapeutic ligands conjugated to reporter oligonucleotides is derived. In some embodiments, a count matrix of cells expressing mutated therapeutic antibodies, drug-reactive antibodies conjugated to reporter oligonucleotides, and therapeutic ligands conjugated to reporter oligonucleotides is derived. The count matrix provides data regarding cells (e.g., T cells or B cells) from a composition that containing antibodies that can bind to a therapeutic antibody, antibody-drug conjugate, control antigen, viral antigen, or other etiologic antigen, such as a cancer antigen, control antigen, or decoy antigen.

In some embodiments, cells are filtered to retain cells that have both a detected VDJ/antibody sequence and non-zero drug-reactive antibody counts and sequenced to an appropriate depth. In some embodiments, cells are sequenced at approximately 5,000 to 100,000 reads per cell for both antigen and VDJ libraries. In some embodiments, cells are sequenced at approximately 5,000 to 20,000 reads per cell for both antigen and VDJ libraries. In some embodiments, cells are sequenced at approximately 20,000 to 40,000 reads per cell for both antigen and VDJ libraries. In some embodiments, cells are sequenced at approximately 40,000 to 60,000 reads per cell for both antigen and VDJ libraries. In some embodiments, cells are sequenced at approximately 60,000 to 80,000 reads per cell for both antigen and VDJ libraries. In some embodiments, cells are sequenced at approximately 80,000 to 100,000 reads per cell for both antigen and VDJ libraries. In some embodiments, saturation can occur rapidly.

The count matrix is retained from cells that are removed. For each antigen, background concentration and ambient concentration are calculated (e.g., mean, median, or hypergeometric mean), and appropriate deviation for non-cell-associated (empty barcodes) is calculated. For a given cell retained by the filter, a logarithm (e.g., natural, base 10, or base 2) or similar transformation (e.g., variance stabilizing transformation, square root, or cubic root) is performed, a pseudocount (e.g., ranging from 1-30) is added, and the background is subtracted. The value is scaled (e.g., by division) by the deviation or variance of the given antigen. Further, the value is modified using a Gaussian mixture model for each antigen on a count vector for each cell, and can include, for example, but not limited to, covariates for sequencing depth or number of detected unique molecular identifiers, cell annotations, or other technical or biological covariates, such as the expression level of the BCR within the cell.

Alternatively, in some embodiments, background and/or ambient concentration is calculated in empty droplets, and subtracted for each antigen. A pseudocount is added (e.g., ranging from 1-50) in some embodiments. The center log ratio or other appropriate compositional transformation can then be applied.

In some embodiments, the resulting matrix and cellular populations thereof are visualized using standard dimensional reduction models, for example PCA, UMAP, t-SNE, or Poincare disks. Further application of spectral clustering, granularity/resolution-based/community detections algorithms, or other algorithms are used to separate cells into groups of cells with distinct drug reactive antibody profiles.

### Example 2: Enzyme-Tagged Therapeutic Antibodies or Antibody-Drug Conjugates

Methods provided herein can be used for enzyme-tagged therapeutic antibodies or antibody-drug conjugates, wherein the therapeutic antibodies or antibody-drug conjugates are tagged for example with a sortase motif (LPXTG). A therapeutic antibody or antibody-drug conjugate is bound by a cell expressing an anti-drug antibody and captured. Incubation with a protein tag or peptide tag including an N-terminal glycine and addition of a bacterial SrtA protein or an engineered variant thereof (e.g., P94, D160N, or K196T) confers a protein tag which is recognized by a barcoded antibody with an additional round of staining. For example, the covalently or non-covalently attached protein tag is selected from BCCP (biotin carboxyl carrier protein) tag, glutathione-S-transferase tag, green fluorescent protein tag, halo-tag, SNAP tag, CLIP tag, HUH tag, maltose binding protein tag, Nus tag, thioredoxin tag, Fc tag, and CRDSAT tag. For example, the covalently or non-covalently attached peptide tag is selected from ALFA tag, AviTag, C-tag, calmodulin tag, polyglutamate tag, polyarginine tag, E tag, FLAG tag, HA tag, His tag, Myc tag, NE tag, Rho1D4 tag, S tag, SBP tag, Softag 1, Softag 3, Spot tag, Strep tag, T7 tag, TC tag, Ty tag, V5 tag, VSV tag, Xpress tag, Isopeptag, Spy tag, Snoop tag, DogTag, and SdyTag.

### Example 3: Screening Anti-Drug Antibodies Using a Cell Bead

In some embodiments, a plasma cell contained within a cell bead gel bead with a matrix capturing human IgG constant regions is screened by tethering a secreted putative anti-drug antibody to the cell bead. An antigen is then used to detect anti-drug antibodies on the surface of the cell bead, which is then isolated to detect an anti-drug antibody sequence. In some embodiments, plasma cells incubated with bi-specific or fusion anti-CD45+anti-Ig constant region antibodies with their own reporter barcodes are used to capture and/or tether a secreted putative anti-drug antibody to the surface of the cell for further screening.

### Example 4: Screening Anti-Drug Antibodies Using a Biotin Conjugated Antigen in a Cell Bead

In some embodiments, an antigen is conjugated (directly or indirectly) to biotin, and a cell bead is magnetized or coated with a magnetic-sensitive matrix. The magnetized cell bead is pulled through one or more fluidic streams comprising free streptavidin (e.g., on a microfluidic chip). Such streams wash away excess antigen while pulling the cell bead gel bead toward an electrode. In some embodiments, a droplet is used in place of a cell bead.

### Example 5: Capture of Anti-Drug Antibodies Using a Splint Oligonucleotide

In some embodiments, a splint oligonucleotide is used to capture and/or detect both an anti-drug antibody RNA transcript and genomic DNA. In some embodiments, RTX polymerase is used to produce a clonable and uniquely amplifiable anti-drug antibody VDJ sequence that is cell associated. Such sequences are amplified after next-generation sequencing and analysis, and can enable rapid production of anti-drug antibodies for research or screening purposes.

### Example 6: Isolation of Antigen Binding Molecules Using a Series of Droplet Generators and Electrodes

A series of droplet generators and electrodes are connected to B cells or putative anti-drug antibody expressing cells are partitioned and incubated. For example, a series of droplet generators and electrodes are used to partition and incubate B cells or putative anti-drug antibody expressing cells. In some embodiments, incubation allows expression of the anti-drug antibody. Cells are passed through a second junction where antigen-expressing cells or antigens are placed into droplets or cell bead gel beads with putative anti-drug antibody expressing cells, or into a chamber where electrodes are used to merge droplets of the two, co-incubated over time, and then passed through a third microfluidic channel, incubated to allow sufficient time for an enzymatic reaction to occur (e.g., 3-20 hours), and screened.

### Example 7: Efficacy Validation and Re-Engineering Therapeutic Antibodies and Antibody Drug Conjugates

Anti-drug antibodies isolated using a method provided herein can be used to validate the efficacy of a therapeutic antibody or antibody-drug conjugate re-engineering. By expressing and labeling the anti-drug antibodies with oligonucleotides and staining engineered cells expressing mutants of the therapeutic antibody or antibody drug conjugate of choice, the workflow described in example 1 is used to confirm that the new therapeutic antibody or antibody-drug variant is not bound by the same antibodies as the original therapeutic antibody or antibody-drug variant. Furthermore, use of such antibodies in functional assays (e.g., ADCC, ADCP, ADCD, or other assays) are used to identify anti-drug antibodies with therapeutic value. For example, such anti-drug antibodies are developed as drugs to be administered to patients experiencing side effects induced by a therapeutic antibody or antibody-drug conjugate by neutralizing the drug or promoting its destruction. Such anti-drug antibodies can also be deployed as reference standards in traditional serum-based anti-drug antibody assays. In some embodiments, this can be effective for therapeutic antibodies or antibody-drug conjugates that have been configured to be recycled efficiently, such as by the addition of one or more Fc domains that are recognized by a neonatal Fc receptor (FcRn, product of the FCGRT gene). Antigens more broadly can also be used to characterize the response in patients with diverse immunoglobulin haplotypes, and in combination with traditional statistical models such as contingency tests, GWAS, PheWAS, and/or cellular assays discover genomic or other elements associated with the development of anti-drug antibodies.

### Example 8: Identification of Re-Engineered Antigens Recognized by Antigen Binding Molecules

The methods provided herein can be implemented to perform an experiment with 5 different point mutants of adalimumab (FIG. 16). Following the procedure of the method, each point mutant is labeled with its own unique reporter barcode oligonucleotide population. The presence of 2 distinct populations of single cells in the resulting clustering and visualization analyses indicates that these populations of cells can recognize distinct (e.g., disjoint) sets of the 5 antigens used (FIG. 17). Mutation or masking of the identified amino acids in the parent antigen (i.e., adalimumab) ablates recognition of the drug by patient derived anti-drug antibodies, guiding the drug engineering and development process accordingly. Optionally, a re-engineered antibody is knocked into a cell line, expressed, and purified to produce a new antigen (e.g., adalimumab variant) to be used in the same patient samples.

### Example 9: Analysis of Anti-Drug Antibodies (ADA) or Drug-Reactive Antibodies (DRA)

The methods provided herein can be implemented to isolate and characterize anti-drug antibodies (ADAs) or drug-reactive antibodies (DRAs) via barcoding. For example, an antigen conjugated to a reporter molecule is used to profile samples from a subject treated with an antibody-drug conjugate (ADC) and/or an antibody therapeutic (AbTx). An exemplary workflow for associating the reporter molecule with an antibody (e.g., an ADA, an ADC, or an AbTx) is shown in FIG. 21. For example, the reporter molecule 2120 is conjugated with e.g., an antibody-binding protein (e.g., Protein A or Protein G), and the complex 2130 is formed by non-covalent association between the antibody binding protein and the ADC and/or the AbTX. The complex 2130 is incubated with cells. In some embodiments, the cells are isolated from a subject. In alternative embodiments, the cells express an AbTx which is unmodified or modified. Unbound 2130 is washed off, and 2140 is partitioned, e.g., into a droplet 2150 for further analysis.

### Example 10: Identification of Anti-Drug Antibodies (ADAs)

The methods provided herein can be implemented to identify ADAs to an AbTx via barcoding. An exemplary workflow of identifying ADAs is described in FIG. 24A. A sample is obtained from a patient treated with a known AbTx. For example, peripheral blood mononuclear cells (PBMCs) are isolated from the patient treated with the AbTx.

Alternatively, a cellular yeast or mammalian cell display library expressing a library of antibodies are used in place of PBMCs. This display library can comprise a library of antibodies derived from the patient treated with the AbTx.

When the sample is the isolated PBMCs, the sample can be enriched for B cells or T cells with a commercially available kit. An exemplary protocol for B cell enrichment is as following: (1) centrifuge a vial of the PBMCs isolated from the patient dispersed in 10% Fetal Bovine Serum (FBS) in PBS to obtain a cell pellet; (2) wash the cell pellet three times by resuspending in 0.04% Bovine Serum Albumin (BSA) in PBS; (3) resuspend the final pellet to a concentration of ˜20 million cells per mL in a total volume of 5 mL (˜100 million cells total); (4) enrich B cells using the B Cell Isolation Kit II (human; MACS™ Miltenyi) according to manufacturer's instructions; (5) apply ˜50 million cells to each of two LS columns designed for positive selection of cells. The effluent is concentrated and prepared for cell labeling.

The known AbTx is conjugated to a reporter oligonucleotide according to the methods described herein. In some embodiments, the AbTx is also labeled with a reporter fluorophore or any other detectable labels that can enable identification of the AbTx by, for example, flow cytometry or any other known similar methods. In some embodiments, antigen-specific enrichment of B cells is performed using flow cytometry. In some embodiments, the AbTx is pre-incubated with its labeled antigenic target (AgTx), enabling identification of ADAs binding to AbTx-AgTx complex rather than the AbTx alone.

The cells are incubated with the reporter oligonucleotide conjugated AbTx. In some embodiments, the cells are contacted with a reporter oligonucleotide conjugated AbTx panel comprising unmodified and modified (or mutated) AbTx conjugated with reporter oligonucleotides. The cells bound to the reporter oligonucleotide conjugated AbTx are partitioned and subjected to, for example, 10×5′V2 Single Cell Immune Profiling kit per manufacturer's instructions. Additional information in this regard is found at “support.10×genomics.com/permalink/getting-started-immune-profiling-feature-barcoding.” This generates a reporter barcode library, a V(D)J library, and optional a gene expression library. The reporter barcode library includes counts of AbTx, AgTx, and/or ADA. The V(D)J library includes native sequences of ADAs or expressed antibody sequences in engineered cells.

The generated libraries are sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®).

A count matrix of cells expressing ADAs and reporter barcodes associated with the AbTx or AbTx-AgTx complex is generated from the sequence data. In some embodiments, the count matrix is a count matrix described in FIG. 16, wherein each row corresponds to a cell expressing an antigen binding molecule (i.e., an ADA), and each column corresponds to an antigen (i.e., the known AbTx (unmodified), one or more modified (mutated) AbTx with at least one point mutation, or the AbTx-AgTx complex). Each element of the count matrix described in FIG. 16 corresponds to a count of quantity of times the antigen bound to a cell expressing a particular antigen binding molecule (i.e., a particular ADA). BCR or TCR sequences from cells having a threshold number of AbTx reporter oligonucleotide UMI counts are used to identify the particular ADA bound to the unmodified AbTx, modified AbTx, or the AbTx-AgTx complex.

### Example 11: Identification of a Patient Specific Engineered Antibody Therapeutic (AbTx)

The methods provided herein can be implemented to identify a patient specific engineered AbTx that does not elicit an immune response in the patient. An exemplary workflow of identifying the patient specific AbTx is described in FIG. 24B.

The ADAs identified in Example 10 are conjugated to reporter oligonucleotides as described herein. In some embodiments, the ADAs can also be labeled with a reporter fluorophore or any other detectable labels that can enable identification of the ADA by, for example flow cytometry or any other known similar methods.

Cells are engineered to express the AbTx and/or the AbTx comprising one or more point mutations. In some embodiments, the AbTx and/or the modified (mutated) AbTx are tethered to the cell surface when expressed from constructs comprising the 5th exon, but not the 6th exon, of an IgG1 constant region. In some embodiments, the AbTx and/or the modified (mutated) AbTx are secreted when expressed from constructs comprising the 6th exon, but not the 5th exon, of an IgG1 constant region. In some embodiments, the engineered cells expressing the AbTx or the modified (mutated) AbTx are pre-incubated with its labeled antigenic target (AgTx), thereby forming a AbTx-AgTx complex. In some embodiments, the engineered cells are enriched in order to identify a modified (mutated) AbTx with a low ADA activity or a high ADA activity.

The engineered cells are incubated with the reporter oligonucleotide conjugated ADAs. In some embodiments, the engineered cells are contacted with a panel of reporter oligonucleotide conjugated ADAs and a control antibody.

The engineered cells bound to the reporter oligonucleotide conjugated ADAs are partitioned and subjected to, for example, 10×5′V2 Single Cell Immune Profiling kit per manufacturer's instructions. Additional information in this regard can be found at “support.10×genomics.com/permalink/getting-started-immune-profiling-feature-barcoding.” This can generate a reporter barcode library, a V(D)J library, and optional a gene expression library. The AbTx or modified (mutated) AbTx are detected by V(D)J chemistry or by targeted GEX chemistry. Reporter barcoded ADAs are detected by the reporter barcode chemistry. Reporter barcoded AbTx-AgTx can be detected by the reporter barcode chemistry.

Optionally, the libraries are enriched for targets of interest, which includes any combination of ADA antibody sequences identified using methods disclosed herein, and known AbTx or AbTx mutant sequences. An exemplary target enrichment method may comprise providing a plurality of barcoded nucleic acid molecules (e.g., library members) and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

The generated libraries are sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®). The binding affinity of ADAs to the AbTx, the modified (mutated) AbTx, or the AbTx-AgTx complex are determined based on quantity/numbers of unique molecular identifiers (UMIs) associated with each of the antigen binding molecules bound to the target antigen.

A count matrix of cells expressing the AbTx or the modified (mutated) AbTx and reporter barcodes associated with the ADAs is generated. In some embodiments, the count matrix is a count matrix described in FIG. 22, wherein each row corresponds to a cell expressing an antigen binding molecule (i.e., the known AbTx (unmodified), one or more modified (mutated) AbTx with at least one point mutation, or the AbTx-AgTx complex), and each column corresponds to an antigen (i.e., an ADA). Each element of the count matrix described in FIG. 22 corresponds to a count of quantity of times the antigen bound to a cell expressing a particular antigen binding molecule (i.e., a particular AbTx). This data is used to identify the epitope (the antigen binding site) of the unmodified AbTx, the modified (mutated) AbTx, or the AbTx-AgTx bound by the ADA. This data is also used to identify a patient-specific AbTx, engineered to not elicit an immune response in the patient when treated with the identified patient-specific AbTx.

### Example 12: Characterization of Anti-Drug Antibody (ADA) Binding to Antibody-Therapeutic (AbTx)

The methods provided herein can be implemented to identify a patient specific engineered AbTx that does not elicit an immune response in patient. In some cases, the methods identify a patient-specific engineered AbTx that retains its binding profile to its AgTx and does not elicit an immune response in the patient. Exemplary workflows of identifying the patient specific AbTx are described in FIGS. 24C-24D.

Referring to FIG. 24C, the AbTx is conjugated to reporter oligonucleotides as described herein. Optionally, the AbTx is modified (mutated) to comprise at least one mutation, wherein each modified AbTx is conjugated with a unique reporter oligonucleotide. For example, a panel of AbTx's, e.g., including an unmodified AbTx and one or more modified AbTx's, can be conjugated with reporter oligonucleotides that identify the AbTx. The AbTx and/or modified AbTx is also labeled with a reporter fluorophore or any other detectable labels that can enable identification of the AbTx by, for example flow cytometry or any other known similar methods. In some embodiments, the AbTx and/or modified AbTx is labeled with an enrichment molecule, which can, for example, without limitation, activate FRET-based fluorescence upon binding its target ligand. In some embodiments, the target ligand is labeled with a reporter oligonucleotide or a fluorescent molecule that can be used in downstream enrichment steps.

Continuing with FIG. 24C, cells are engineered to express the ADAs identified in Example 10. Optionally, cells engineered to express a control antibody are provided. The control antibody is a non-ADA that binds an irrelevant target that is not the AbTx (e.g., a cell surface protein, a murine version of a human protein). The control antibodies are a commercially available clone against, for example, without limitation, CD3 or CD8. In some embodiments, the ADAs are tethered to the cell surface when expressed from constructs comprising the 5th exon, but not the 6th exon, of an IgG1 constant region. In some embodiments, the ADAs are secreted when expressed from constructs comprising the 6th exon, but not the 5th exon, of an IgG1 constant region.

The engineered cells are incubated with the AbTx and/or the modified (mutated) AbTx conjugated with reporter oligonucleotides. In some embodiments, the engineered cells are contacted with a panel of the AbTx and/or the modified (mutated) AbTx conjugated with reporter oligonucleotides. In some embodiments, the engineered cells expressing the ADAs are pre-incubated with its labeled antigenic target, which can optionally comprise a reporter oligonucleotide that can identify the antigenic target. In some embodiments, the engineered cells are enriched in order to identify a modified (mutated) AbTx that poorly or strongly binds ADAs. This enrichment process can optionally be repeated in a panning-like process.

The engineered cells bound to the AbTx and/or modified (mutated) AbTx conjugated with reporter oligonucleotides are partitioned and subjected to, for example, 10×5′V2 Single Cell Immune Profiling kit per manufacturer's instructions. Additional information in this regard can be found at “support.10×genomics.com/permalink/getting-started-immune-profiling-feature-barcoding.” This generates a reporter barcode library, a V(D)J library, and optional a gene expression library. The reporter barcode library can be used to detect the presence of the AbTx, the modified (mutated) AbTx, and optionally the AgTx ligand. The V(D)J library and the gene expression library are used to detect the presence of ADAs and the control antibodies expressed by the cells. Optionally, the libraries are enriched, e.g., according to the methods described in Example 11.

The generated libraries are sequenced on, for example, a NovaSeq 3 using a NovaSeq S4 200 cycles 2020 v1.5 kit per manufacturer's instructions. Sequencing can also be performed by various systems currently available, such as, without limitation, a sequencing system by Illumina®, Pacific Biosciences (PacBio®), Oxford Nanopore®, or Life Technologies (Ion Torrent®). The binding affinity of ADAs to the AbTx, the modified (mutated) AbTx, or the AbTx-AgTx complex are determined based on quantity/numbers of unique molecular identifiers (UMIs) associated with each of the antigen binding molecules bound to the target antigen.

A count matrix of cells expressing the ADAs and the AbTx or the modified (mutated) AbTx conjugated with and reporter barcodes are generated. In some embodiments, the count matrix is a count matrix described in FIG. 23, wherein each row corresponds to a cell expressing an antigen binding molecule (i.e., the known AbTx (unmodified), one or more modified (mutated) AbTx with at least one point mutation, or the AbTx-AgTx complex), and each column corresponds to an antigen (i.e., an ADA). Each element of the count matrix described in FIG. 23 corresponds to a count of quantity of times the antigen bound to a cell expressing a particular antigen binding molecule (i.e., a particular AbTx). This data can be used to identify (1) cases where each AbTx or each modified (mutated) AbTx is bound by one or more ADAs; (2) whether each AbTx or each modified (mutated) AbTx binds the AgTx ligand; (3) whether an ADA binding of each AbTx or each modified (mutated) AbTx can abrogate AgTx ligand binding; and (4) whether a particular AbTx-ADA binding is dependent on a AgTx binding to AbTx.

The ADAs and AbTx can be reversed in this workflow, where a library of ADAs binds the cells expressing the AbTx and/or the modified (mutated) AbTx, the process of which is described in FIG. 24D.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. (canceled)

2. A method of identifying at least one antigen binding molecule that binds an antigen, the method comprising:
(a) contacting (i) a composition comprising one or more cells expressing the at least one antigen binding molecule with (ii) a reporter oligonucleotide conjugated antigen, wherein the reporter oligonucleotide comprises a reporter barcode and the antigen is an antibody therapeutic or an antibody drug conjugate (ADC);
(b) sequencing the at least one antigen binding molecule; and
(c) identifying the at least one antigen binding molecule.

3. The method of claim 2, further comprising generating a plurality of partitions, wherein a partition of the plurality of partitions comprises a single cell and a partition specific barcode molecule comprising a partition specific barcode sequence after step (a) and before step (b).

4. The method of claim 2, further comprising:
(d) measuring a number of cells that express the at least one antigen binding molecule that bind to the antigen to quantify a subject's response to the antigen, wherein the one or more cells is obtained from the subject.

5. The method of claim 2, further comprising:
(d) determining diversity of a subject's immune response to the antibody therapeutic by identifying the at least one antigen binding molecule that binds to the antigen, wherein the one or more cells is obtained from the subject.

6-7. (canceled)

8. The method of claim 2, comprising:
(d) monitoring a subject's response to the antigen by measuring a number of cells expressing the at least one antigen binding molecule that bind to the antigen over a course of time, wherein the one or more cells is obtained from the subject.

9. (canceled)

10. The method of claim 8, wherein the course of time is about bi-weekly, about monthly, or about yearly.

11. The method of claim 2, wherein the one or more cells comprises at least one NK cell, or at least one B cell.

12. (canceled)

13. The method of claim 2, wherein the one or more cells are obtained from a subject previously treated with the antibody therapeutic or the ADC.

14. The method of claim 2 wherein the at least one antigen binding molecule comprises an antibody or antigen binding fragment thereof.

15. The method of claim 14, or wherein the sequencing in step (b) comprises determining all or a part of the sequence of the antibody or antigen binding fragment thereof.

16. The method of claim 15, wherein the antibody or antigen binding fragment thereof is an anti-SARS-CoV-2 antibody or antigen binding fragment thereof.

17-22. (canceled)

23. The method of claim 2, wherein the antigen is a component of a vaccine or a chimeric antigen receptor.

24. (canceled)

25. The method of claim 23, wherein the chimeric antigen receptor is selected from the group consisting of axicabtagene ciloleucel (YESCARTA®), tisagenlecleucel (KYMRIAH®), and brexucabtagene autoleucel (TECARTUS).

26. The method of claim 2, wherein the reporter oligonucleotide conjugated antigen further comprises an enzyme tag, a fluorophore tag, a quantum dot, a covalently or non-covalently attached protein tag, a covalently or non-covalently attached peptide tag, a fused tag, a carbohydrate tag, or a small molecule tag.

27. The method of claim 26, wherein the covalently or non-covalently attached protein tag is selected from BCCP (biotin carboxyl carrier protein) tag, glutathione-S-transferase tag, green fluorescent protein tag, halo-tag, SNAP tag, CLIP tag, HUH tag, maltose binding protein tag, Nus tag, thioredoxin tag, Fc tag, and CRDSAT tag; and wherein the covalently or non-covalently attached peptide tag is selected from ALFA tag, AviTag, C-tag, calmodulin tag, polyglutamate tag, polyarginine tag, E tag, FLAG tag, HA tag, His tag, Myc tag, NE tag, Rho1D4 tag, S tag, SBP tag, Softag 1, Softag 3, Spot tag, Strep tag, T7 tag, TC tag, Ty tag, V5 tag, VSV tag, Xpress tag, Isopeptag, Spy tag, Snoop tag, DogTag, and SdyTag.

28. (canceled)

29. The method of claim 2, wherein the reporter oligonucleotide is conjugated to the antigen via one or more of the following methods: ReACT chemistry, direct/non-specific (lysine) click chemistry, site-specific sortase motif-dependent conjugation, site-specific photo-crosslinking-dependent conjugation, site-specific conformation-dependent conjugation, and nitrilotriacetate conjugation.

30-37. (canceled)

38. The method of claim 2, wherein the reporter oligonucleotide is captured by contacting the reporter oligonucleotide comprising the reporter barcode to a partition-specific barcode molecule, optionally wherein the contacting comprises hybridizing the reporter oligonucleotide to the partition-specific barcode molecule, wherein the partition-specific barcode molecule comprises a partition-specific barcode, a unique molecular identifier (UMI), and/or a template switching oligonucleotide (TSO) site.

39-49. (canceled)

50. The method of claim 2 wherein the antibody therapeutic is selected from the group consisting of abciximab, adalimumab, adalimumab-atto, ado-trastuzumab emtansine, alemtuzumab, alirocumab, atezolizumab, avelumab, basiliximab, belimumab, bevacizumab, bezlotoxumab, blinatumomab, brentuximab vedotin, brodalumab, canakinumab, capromab pendetide, certolizumab pegol, cetuximab, daclizumab, daratumumab, denosumab, dinutuximab, dupilumab, durvalumab, eculizumab, elotuzumab, evolocumab, golimumab, ibritumomab tiuxetan, idarucizumab, infliximab, infliximab-abda, infliximab dyyb, ipilimumab, ixekizumab, mepolizumab, natalizumab, necitumumab, nivolumab, oblitoxaximab, obinutuzumab, ocrelizumab, ofatumumab, olaratumab, omalizumab, palivizumab, panitumumab, pembrolizumab, pertuzumab, ramucirumab, ranibizumab, raxibacumab, reslizumab, rituximab, secukinumab, siltuximab, tocilizumab, trastuzumab, ustekinumab, vedolizumab, sarilumab, guselkumab, inotuzumab ozogamicin, adalimumab-adbm, gemtuzumab ozogamicin, bevacizumab-awwb, benralizumab, emicizumab-kxwh, trastuzumab-dkst, infliximab-qbtx, ibalizumab-uiyk, tildrakizumab-asmn, burosumab-twsa, erenumab-aooe, tositumomab, mogamulizumab, moxetumomab pasudotox, cempilimab, and polatuzumab vedotin.

51. The method of claim 2, further comprising:
after the contacting in step (a), (i) identifying the one or more cells binding to the reporter oligonucleotide conjugated antigen using the reporter barcode, and optionally isolating the one or more cells binding to the reporter oligonucleotide conjugated antigen; and
(ii) using the binding to generate a count matrix comprising information for (1) the binding cell counts and/or (2) unique molecular identifier (UMI) counts.

52. The method of claim 51, further comprising
(iii) embedding, in a lower dimensional space, the count matrix, the embedding includes transforming the count matrix by applying one or more of a log-transformation, a variance-stabilizing transformation, a square root transformation, and a cubic root transformation; and
(iv) generating, based at least on the embedded count matrix, to identify one or more distinct populations, wherein each population of the one or more distinct populations represents a similar binding profile.

53. The method of claim 13, further comprising:
using the reporter barcode to identify the one or more cells binding to the reporter oligonucleotide conjugated antigen, and optionally isolating the one or more cells binding to the reporter oligonucleotide conjugated antigen, to identify and/or to isolate an anti-drug antibody.

54. The method of claim 0, further comprising identifying an antigen binding site of the antibody therapeutic or the ADC to which the anti-drug antibody binds, and optionally modifying the antigen binding site of the antibody therapeutic or the ADC to modify the binding of the antibody therapeutic or the ADC to the anti-drug antibody.

55. (canceled)

56. A method of identifying an antibody therapeutic that does not elicit an immune response, the method comprising:
(a) contacting a composition comprising one or more cells from a subject treated with the antibody therapeutic to
(i) a control reporter oligonucleotide conjugated antigen, wherein the control reporter oligonucleotide comprises a first reporter barcode and the antigen is the antibody therapeutic, and
(ii) at least one test reporter oligonucleotide conjugated antigen, wherein the test reporter oligonucleotide comprises a second reporter barcode and the antigen is a modified version of the antibody therapeutic; and
(b) using the first and second reporter barcodes to identify the one or more cells binding to (i) and/or (ii), and optionally isolating the one or more cells binding to (i) and/or (ii); and
(c) identifying a test reporter oligonucleotide conjugated antigen of the at least one test reporter oligonucleotide conjugated antigen, wherein the test reporter oligonucleotide conjugated antigen has less binding as compared to the control reporter oligonucleotide conjugated antigen.

57. (canceled)

58. The method of claim 56, further comprising
(d) using the binding to generate a count matrix comprising information for (1) the binding cell counts and/or (2) unique molecular identifier (UMI) counts.

59. The method of claim 58, further comprising
(e) embedding, in a lower dimensional space, the count matrix, the embedding includes transforming the count matrix by applying one or more of a log-transformation, a variance-stabilizing transformation, a square root transformation, and a cubic root transformation; and
(f) generating, based at least on the embedded count matrix, to identify one or more distinct populations, wherein each population of the one or more distinct populations represents a similar binding profile.

60. The method of claim 56, wherein the modified version of the antibody therapeutic comprises at least one point mutation.

61. The method of claim 56, further comprising identifying an antigen binding site in which the modified version of the antibody therapeutic binds.

62. The method of claim 56, wherein the modified version of the antibody therapeutic is used or is capable of being used as a subject-specific antibody therapeutic.

63. (canceled)

64. A system, comprising:
at least one data processor; and
at least one memory storing instructions, which when executed by the at least one data processor, result in operations comprising:
generating, based at least on a reporter oligonucleotide conjugated to each of a plurality of antigens, a count matrix indicating a count of a quantity of times each of the plurality of antigens bound to each of a plurality of cells expressing one or more antigen binding molecules;
embedding, in a lower dimensional space, the count matrix; and
identifying, based at least on the embedded count matrix, one or more distinct populations of cells expressing one or more antigen binding molecules, each of the one or more distinct populations of cells expressing one or more antigen binding molecules capable of binding to one or more of a same antigen.

65-75. (canceled)

76. The system of claim 64, wherein the operations further comprise:
determining an ambient concentration of each of the plurality of antigens and/or the reporter oligonucleotide conjugated to each of the plurality of antigens; and
subtracting, from the count matrix, the ambient concentration prior to embedding the count matrix.

77. The system of claim 64, wherein the operations further comprise: filtering the count matrix to at least retain one or more cells expressing one or more antigen binding molecules having (i) a detected variability, diversity, and joining (VDJ) sequence and/or an antibody sequence, (ii) a non-zero binding count, and (iii) a sufficient sequencing depth.

78. The system of claim 64, wherein the operations further comprise: generating a visualization of the one or more distinct populations of cells expressing one or more antigen binding molecules.

79-81. (canceled)

82. A computer-implemented method, comprising:
generating, based at least on a reporter oligonucleotide conjugated to each of a plurality of antigens, a count matrix indicating a count of a quantity of times each of the plurality of antigens bound to each of a plurality of cells expressing one or more antigen binding molecules;
embedding, in a lower dimensional space, the count matrix; and
identifying, based at least on the embedded count matrix, one or more distinct populations of cells expressing one or more antigen binding molecules, each of the one or more distinct populations of cells expressing one or more antigen binding molecules capable of binding to one or more of a same antigen.

83-101. (canceled)
